(19) United States
(12) Patent Application Publication
(10) Pub. No.: US 2022/0112187 A1
(43) Pub. Date: Apr. 14, 2022

(54) PYRAZOLOPYRIDINE AMINE COMPOUNDS FOR THE TREATMENT OF AUTOIMMUNE DISEASE

(71) Applicant: Hoffmann-La Roche Inc., Little Falls, NJ (US)

(72) Inventors: Haixia LIU, Shanghai (CN); Hong SHEN, Shanghai (CN); Wei ZHU, Shanghai (CN); Taishan HU, Shanghai (CN); Zhiwei ZHANG, Shanghai (CN); Fabian DEY, Basel (CH)

(73) Assignee: Hoffmann-La Roche Inc., Little Falls, NJ (US)

(21) Appl. No.: 17/275,498

(22) PCT Filed: Sep. 11, 2018
(86) PCT No.: PCT/EP2018/074463

§ 371 Date: Mar. 11, 2021

(51) Int. Cl. C07D 471/04; C07D 519/00; A61P 37/06

(52) U.S. Cl. CPC C07D 471/04; A61P 37/06; C07D 519/00

(57) ABSTRACT

The present invention relates to compounds of formula (I), wherein R¹, R², R³, R⁴ and X are as described herein, and their pharmaceutically acceptable salt, enantiomer or diastereomer thereof, and compositions including the compounds and methods of using the compounds.

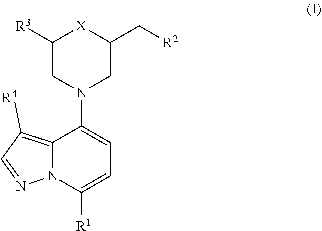

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SEE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SEE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immuno suppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721.). Other biologies, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7/8/9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1.) Therefore, TLR7/8/9 represents a new therapeutic target for autoimmune and auto-inflammatory diseases, for which no effective steroid-free and non-cytotoxic oral drugs exist, and inhibition of these pathways from the very upstream may deliver satisfying therapeutic effects. From a safety perspective, because there are multiple nucleic acid sensing pathways (e.g. other TLRs, cGAS/STING), such redundancy should still allow responses to infection in the presence of TLR7/8/9 inhibition. As such, we proposed and invented oral compounds that target and suppress TLR7/8/9 for the treatment of autoimmune and auto-inflammatory diseases.

### SUMMARY OF THE INVENTION

The present invention relates to novel compounds of formula (I),

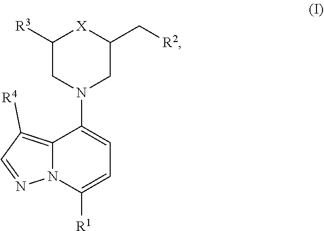

wherein

R¹ is cyano, C₁₋₆alkyl, halogen, haloC₁₋₆alkyl or nitro;
R² is heterocyclyl or heterocyclylamino;
R³ is C₁₋₆alkyl or haloC₁₋₆alkyl;
R⁴ is H or halogen;

### X is O or CH₂;

or a pharmaceutically acceptable salt, enantiomer or diastereomer thereof.

Another object of the present invention is related to novel compounds of formula (I), their manufacture, medicaments based on a compound in accordance with the invention and their production as well as the use of compounds of formula (I) as TLR7 and/or TLR8 and/or TLR9 antagonist, and for the treatment or prophylaxis of systemic lupus erythematosus or lupus nephritis. The compounds of formula (I) show superior TLR7 and/or TLR8 and/or TLR9 antagonism activity. In addition, the compounds of formula (I) also show good cytotoxicity, solubility, human micro some stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “halopiperidinyl” denotes a piperidinyl group wherein at least one of the hydrogen atoms of the piperidinyl group has been replaced by same or different halogen atoms, particularly fluoro atoms. Examples of halopiperidinyl include fluoropyrrolidinyl and difluoropiperidinyl.

The term “heterocyclyl” denotes a monovalent saturated or partly unsaturated mono- or bicyclic ring system of 3 to 12 ring atoms, comprising 1, 2, or 3 ring heteroatoms selected from N, O and S, the remaining ring atoms being carbon. In particular embodiments, heterocyclyl is a monovalent saturated monocyclic ring system of 4 to 10 ring atoms, comprising 1, 2, or 3 ring heteroatoms selected from N, O and S, the remaining ring atoms being carbon. Examples for monocyclic saturated heterocyclyl are aziridinyl, oxiranyl, azetidinyl, oxetanyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Heterocyclyl can be fully or partially saturated. Examples for bicyclic saturated heterocyclyl are (3,4,4a,5,6,7,8,8a-octahydro-2H-naphthyridinyl; 3,3a,4,5,6,6a-hexahydro-1H-cyclopenta[c]pyrrolyl; 1,2,3,3a,4,6,7,7a-octahydropyrrolo[3,2-c]pyridinyl; 1,2,3,3a,5,6,7,7a-octahydropyrrolo[3,2-b]pyridinyl; 1,2,3,4,4a,5,7,7a-octahydropyrrolo[3,4-b]pyridinyl; 1,3,3a,4,5,6,7,7a-octahydropyrrolo[3,4-c]pyridinyl; 2,3,3a,4,6,6a-hexahydro-1H-pyrrolo[3,4-c]pyrrolyl; 2,3,4,4a,5,6,7,7a-octahydropyrrolo[3,4-b]pyridinyl; 3,3a,4,5,6,6a-hexahydro-2H-pyrrolo[3,4-b]pyrrolyl; azabicyclo[3.2.1]octanyl; azabicyclo[3.3.1]nonanyl; azaspiro[3.3]heptanyl; oxaazabicyclo[3.3.1]nonanyl; oxadiazaspiro[4.5]decanyl; diazabicyclo[2.2.2]octanyl; diazabicyclo[3.2.1]octanyl; diazabicyclo[4.2.0]octanyl; diazaspiro[2.5]octanyl; diazaspiro[3.3]heptanyl; diazaspiro[3.4]octanyl; diazaspiro[3.5]nonanyl; diazaspiro[3.6]decanyl; diazaspiro[4.4]nonanyl; diazaspiro[4.5]decanyl; diazaspiro[5.5]undecanyl; oxadiazabicyclo[3.3.1]nonanyl; oxadiazaspiro[5.5]undecanyl; and oxodiazaspiro[4.4]nonanyl. Examples for partially saturated heterocyclyl are dihydrofuryl, imidazolinyl, dihydro-oxazolyl, tetrahydropyridinyl, and dihydropyranyl. Monocyclic or bicyclic heterocyclyl can be further substituted by halogen, hydroxy, amino, aminoC₁₋₆alkyl, aminoC₁₋₆alkylcarbonyl, C₁₋₆alkylcarbonylamino, (C₁₋₆alkyl)₂amino, carbamoyl, C₁₋₆alkyl, haloC₁₋₆alkyl, phenyl, phenylC₁₋₆alkyl, or heterocyclyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist of TLR7 and/or TLR8 and/or TLR9

The present invention relates to a compound of formula (I),

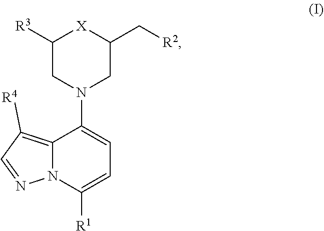

wherein

R¹ is cyano, C₁₋₆alkyl, halogen, haloC₁₋₆alkyl or nitro;
R² is heterocyclyl or heterocyclylamino;
R³ is C₁₋₆alkyl or haloC₁₋₆alkyl;
R⁴ is H or halogen;

### X is O or CH₂;

or a pharmaceutically acceptable salt, enantiomer or diastereomer thereof.

A further embodiment of present invention is (ii) a compound of formula (I) according to (i), wherein

R¹ is cyano;
R² is (3,4,4a,5,6,7,8,8a-octahydro-2H-naphthyridinyl;
(C₁₋₆alkyl)₂amino-3,3a,4,5,6,6a-hexahydro-1H-cyclopenta[c]pyrrolyl;
1,2,3,3a,4,6,7,7a-octahydropyrrolo[3,2-c]pyridinyl;
1,2,3,3a,5,6,7,7a-octahydropyrrolo[3,2-b]pyridinyl;
1,2,3,4,4a,5,7,7a-octahydropyrrolo[3,4-b]pyridinyl;
1,3,3a,4,5,6,7,7a-octahydropyrrolo[3,4-c]pyridinyl;
2,3,3a,4,6,6a-hexahydro-1H-pyrrolo[3,4-c]pyrrolyl;
2,3,4,4a,5,6,7,7a-octahydropyrrolo[3,4-b]pyridinyl;
3,3a,4,5,6,6a-hexahydro-2H-pyrrolo[3,4-b]pyrrolyl;
aminoazabicyclo[3.2.1]octanyl;
aminoazabicyclo[3.3.1]nonanyl;
aminoazaspiro[3.3]heptanyl;
aminooxaazabicyclo[3.3.1]nonanyl;
C₁₋₆alkyloxadiazaspiro[4.5]decanyl;
C₁₋₆alkylpiperidinylamino;
diazabicyclo[2.2.2]octanyl;
diazabicyclo[3.2.1]octanyl;
diazabicyclo[4.2.0]octanyl;
diazaspiro[2.5]octanyl;
diazaspiro[3.3]heptanyl;
diazaspiro[3.4]octanyl;
diazaspiro[3.5]nonanyl;
diazaspiro[3.6]decanyl;
diazaspiro[4.4]nonanyl;
diazaspiro[4.5]decanyl;
diazaspiro[5.5]undecanyl;
oxadiazabicyclo[3.3.1]nonanyl;
oxadiazaspiro[5.5]undecanyl;
oxodiazaspiro[4.4]nonanyl;
piperazinyl, said piperazinyl being unsubstituted or substituted by one, two or three substituents independently selected from C₁₋₆alkyl, phenyl, phenylC₁₋₆alkyl, aminoC₁₋₆alkylcarbonyl and piperidinyl;
piperidinyl, said piperidinyl being unsubstituted or substituted by one, two or three substituents independently selected from amino, aminoC₁₋₆alkyl, azepanyl, C₁₋₆alkyl, C₁₋₆alkylcarbonylamino, C₁₋₆alkylpiperazinyl, carbamoyl, halogen, phenyl, piperazinyl, piperidinyl and pyrrolidinyl; or
pyrrolidinyl, said pyrrolidinyl being unsubstituted or substituted by one, two or three substituents independently selected from amino and halogen;
   R³ is C₁₋₆alkyl;
   R⁴ is H or halogen;

### X is O;

or a pharmaceutically acceptable salt, enantiomer or diastereomer thereof.

A further embodiment of present invention is (iii) a compound of formula (I) according to (ii), wherein

R¹ is cyano;
R² is (3,4,4a,5,6,7,8,8a-octahydro-2H-naphthyridinyl;
2,2-dimethylpropanoylamino-3,3a,4,5,6,6a-hexahydro-1H-cyclopenta[c]pyrrolyl;
1,2,3,3a,4,6,7,7a-octahydropyrrolo[3,2-c]pyridinyl;
1,2,3,3a,5,6,7,7a-octahydropyrrolo[3,2-b]pyridinyl;
1,2,3,4,4a,5,7,7a-octahydropyrrolo[3,4-b]pyridinyl;
1,3,3a,4,5,6,7,7a-octahydropyrrolo[3,4-c]pyridinyl;
2,3,3a,4,6,6a-hexahydro-1H-pyrrolo[3,4-c]pyrrolyl;
2,3,4,4a,5,6,7,7a-octahydropyrrolo[3,4-b]pyridinyl;
3,3a,4,5,6,6a-hexahydro-2H-pyrrolo[3,4-b]pyrrolyl;
aminoazabicyclo[3.2.1]octanyl;
aminoazabicyclo[3.3.1]nonanyl;
aminoazaspiro[3.3]heptanyl;
aminooxaazabicyclo[3.3.1]nonanyl;
methyloxadiazaspiro[4.5]decanyl;
methylpiperidinylamino;
diazabicyclo[2.2.2]octanyl;
diazabicyclo[3.2.1]octanyl;
diazabicyclo[4.2.0]octanyl;
diazaspiro[2.5]octanyl;
diazaspiro[3.3]heptanyl;
diazaspiro[3.4]octanyl;
diazaspiro[3.5]nonanyl;
diazaspiro[3.6]decanyl;
diazaspiro[4.4]nonanyl;
diazaspiro[4.5]decanyl;
diazaspiro[5.5]undecanyl;
oxadiazabicyclo[3.3.1]nonanyl;
oxadiazaspiro[5.5]undecanyl;
oxodiazaspiro[4.4]nonanyl;
piperazinyl, said piperazinyl being unsubstituted or substituted by one, two or three substituents independently selected from methyl, phenyl, benzyl, aminoacetyl and piperidinyl;
piperidinyl, said piperidinyl being unsubstituted or substituted by one, two or three substituents independently selected from amino, aminomethyl, aminoethyl, azepanyl, methyl, 2,2-dimethylpropanoylamino, methylpiperazinyl, carbamoyl, fluoro, phenyl, piperazinyl, piperidinyl and pyrrolidinyl; or
pyrrolidinyl, said pyrrolidinyl being unsubstituted or substituted by one, two or three substituents independently selected from amino and fluoro;
   R³ is methyl;
   R⁴ is H or fluoro;

### X is O;

or a pharmaceutically acceptable salt, enantiomer or diastereomer thereof.

A further embodiment of present invention is (iv) a compound of formula (I) according to (iii), wherein R² is (3,4,4a,5,6,7,8,8a-octahydro-2H-1,5-naphthyridin-1-yl; 1,2,3,3a,4,6,7,7a-octahydropyrrolo[3,2-c]pyridin-5-yl; 1,2,3,3a,5,6,7,7a-octahydropyrrolo[3,2-b]pyridin-4-yl; 1,2,3,4,4a,5,7,7a-octahydropyrrolo[3,4-b]pyridin-6-yl, 1,3,3a,4,5,6,7,7a-octahydropyrrolo[3,4-c]pyridin-2-yl; 1,6-diazaspiro[3.3]heptan-1-yl; 1,7-diazaspiro[3.5]nonan-7-yl; 1,8-diazaspiro[4.5]decan-8-yl; 1,9-diazaspiro[5.5]undecan-9-yl, 1-methyl-4-piperidinylamino; 1-oxa-4,9-diazaspiro[5.5]undecan-4-yl; 1-oxa-4,9-diazaspiro[5.5]undecan-9-yl; 1-oxo-2,7-diazaspiro[4.4]nonan-2-yl; 2-(3-carbamoyl)-1-piperidinyl; 2,3,3a,4,6,6a-hexahydro-1H-pyrrolo[3,4-c]pyrrol-5-yl; 2,3,4,4a,5,6,7,7a-octahydropyrrolo[3,4-b]pyridin-1-yl; 2,5-diazabicyclo[2.2.2]octan-2-yl; 2,6-diazaspiro[3.3]heptan-2-yl; 2,6-diazaspiro[3.5]nonan-6-yl; 2,6-diazaspiro[4.5]decan-2-yl; 2,7-diazaspiro[3.4]octan-2-yl; 2,7-diazaspiro[3.5]nonan-2-yl; 2,7-diazaspiro[3.5]nonan-7-yl; 2,7-diazaspiro[4.4]nonan-2-yl; 2,7-diazaspiro[4.5]decan-7-yl; 2,8-diazaspiro[3.5]nonan-2-yl; 2,8-diazaspiro[3.6]decan-2-yl; 2,8-diazaspiro[4.5]decan-2-yl; 2,8-diazaspiro[4.5]decan-8-yl; 2,8-diazaspiro[5.5]undecan-2-yl; 2,9-diazaspiro[4.5]decan-2-yl; 2,9-diazaspiro[5.5]undecan-2-yl; 2,9-diazaspiro[5.5]undecan-9-yl; 2-benzyl-4-(4-piperidinyl)piperazin-1-yl; 2-benzylpiperazin-1-yl; 2-methylpiperazin-1-yl; 3,3a,4,5,6,6a-hexahydro-2H-pyrrolo[3,4-b]pyrrol-1-yl; 3,7-diazabicyclo[4.2.0]octan-3-yl; 3,7-diazabicyclo[4.2.0]octan-7-yl; 3,8-diazabicyclo[3.2.1]octan-3-yl; 3,8-diazabicyclo[3.2.1]octan-8-yl; 3,8-diazabicyclo[4.2.0]octan-8-yl; 3,9-diazaspiro[5.5]undecan-3-yl; 3-amino-8-azabicyclo[3.2.1]octan-8-yl; 3-amino-9-azabicyclo[3.3.1]nonan-9-yl; 3-methylpiperazin-1-yl; 3-oxa-7,9-diazabicyclo[3.3.1]nonan-9-yl; 3-oxo-2,7-diazaspiro[4.4]nonan-2-yl; 3-phenylpiperazin-1-yl; 4-(1-piperidinyl)-1-piperidinyl; 4-(2,2-dimethylpropanoylamino)-1-piperidinyl; 4-(2-aminoacetyl)piperazinyl; 4-(2-aminoethyl)-1-piperidinyl; 4-(4-methylpiperazin-1-yl)-1-piperidinyl; 4-(aminomethyl)-1-piperidinyl; 4-(azepan-1-yl)-1-piperidinyl; 4-(dimethylamino)-3,3a,4,5,6,6a-hexahydro-1H-cyclopenta[c]pyrrol-2-yl; 4,7-diazaspiro[2.5]octan-7-yl; 4-amino-1-piperidinyl; 4-amino-2-methyl-1-piperidinyl; 4-amino-3,3-difluoro-1-piperidinyl; 4-amino-3,3-difluoro-pyrrolidin-1-yl; 4-amino-3-methyl-1-piperidinyl; 4-amino-4-methyl-1-piperidinyl; 4-amino-4-phenyl-1-piperidinyl; 4-methylpiperazinyl; 4-piperazin-1-yl-1-piperidinyl; 4-pyrrolidin-1-yl-1-piperidinyl; 6-amino-2-azaspiro[3.3]heptan-2-yl; 7-amino-3-oxa-9-azabicyclo[3.3.1]nonan-9-yl; 8-amino-3-azabicyclo[3.2.1]octan-3-yl; 9-methyl-6-oxa-2,9-diazaspiro[4.5]decan-2-yl or piperazinyl.

A further embodiment of present invention is (v) a compound of formula (I) according to (iv), wherein R² is
1,2,3,3a,4,6,7,7a-octahydropyrrolo[3,2-c]pyridinyl;
2,3,3a,4,6,6a-hexahydro-1H-pyrrolo[3,4-c]pyrrolyl;
aminoazabicyclo[3.2.1]octanyl;
aminooxaazabicyclo[3.3.1]nonanyl;
diazabicyclo[2.2.2]octanyl;
diazabicyclo[3.2.1]octanyl;
diazabicyclo[4.2.0]octanyl;
diazaspiro[4.5]decanyl;
oxadiazabicyclo[3.3.1]nonanyl;
oxodiazaspiro[4.4]nonanyl;
piperazinyl, said piperazinyl being unsubstituted or substituted by C₁₋₆alkyl or phenylC₁₋₆alkyl; or
piperidinyl, said piperidinyl being unsubstituted or substituted by one, two or three substituents independently selected from amino, C₁₋₆alkyl, C₁₋₆alkylpiperazinyl and halogen.

A further embodiment of present invention is (vi) a compound of formula (I) according to (v), wherein R² is 1,2,3,3a,4,6,7,7a-octahydropyrrolo[3,2-c]pyridinyl; 2,3,3a,4,6,6a-hexahydro-1H-pyrrolo[3,4-c]pyrrolyl; aminoazabicyclo[3.2.1]octanyl; aminooxaazabicyclo[3.3.1]nonanyl; diazabicyclo[2.2.2]octanyl; diazabicyclo[3.2.1]octanyl; diazabicyclo[4.2.0]octanyl; diazaspiro[4.5]decanyl; oxadiazabicyclo[3.3.1]nonanyl; oxodiazaspiro[4.4]nonanyl; piperazinyl, methylpiperazinyl; benzylpiperazinyl; methylpiperazinylpiperidinyl; aminopiperidinyl; amino(C₁₋₆alkyl)piperidinyl or aminohalopiperidinyl.

A further embodiment of present invention is (vii) a compound of formula (I) according to (vi), wherein R² is 4-(4-methylpiperazin-1-yl)-1-piperidinyl; 1,2,3,3a,4,6,7,7a-octahydropyrrolo[3,2-c]pyridin-5-yl; 1-oxo-2,7-diazaspiro[4.4]nonan-2-yl; 2,3,3a,4,6,6a-hexahydro-1H-pyrrolo[3,4-c]pyrrol-5-yl; 2,5-diazabicyclo[2.2.2]octan-2-yl; 2,6-diazaspiro[4.5]decan-2-yl; 2-benzylpiperazin-1-yl; 3,7-diazabicyclo[4.2.0]octan-7-yl; 3,8-diazabicyclo[3.2.1]octan-3-yl; 3,8-diazabicyclo[3.2.1]octan-8-yl; 3-methylpiperazin-1-yl; 3-oxa-7,9-diazabicyclo[3.3.1]nonan-9-yl; 4-amino-1-piperidinyl; 4-amino-3,3-difluoro-1-piperidinyl; 4-amino-3-methyl-1-piperidinyl; 4-amino-4-methyl-1-piperidinyl; 7-amino-3-oxa-9-azabicyclo[3.3.1]nonan-9-yl; 8-amino-3-azabicyclo[3.2.1]octan-3-yl or piperazinyl.

A further embodiment of present invention is (viii) a compound of formula (I) according to (v) or (vi), wherein R² is C₁₋₆alkylpiperazinylpiperidinyl; 1,2,3,3a,4,6,7,7a-octahydropyrrolo[3,2-c]pyridinyl; oxadiazaspiro[5.5]undecanyl; 2,3,3a,4,6,6a-hexahydro-1H-pyrrolo[3,4-c]pyrrolyl; diazaspiro[4.5]decanyl; diazabicyclo[4.2.0]octanyl; aminopiperidinyl; amino(C₁₋₆alkyl)piperidinyl.

A further embodiment of present invention is (ix) a compound of formula (I) according to (viii), wherein R² is 4-(4-methylpiperazin-1-yl)-1-piperidinyl; 1,2,3,3a,4,6,7,7a-octahydropyrrolo[3,2-c]pyridin-5-yl; 1-oxa-4,9-diazaspiro[5.5]undecan-9-yl; 2,3,3a,4,6,6a-hexahydro-1H-pyrrolo[3,4-c]pyrrol-5-yl; 2,9-diazaspiro[4.5]decan-2-yl; 3,7-diazabicyclo[4.2.0]octan-7-yl; 4-amino-1-piperidinyl; 4-amino-3-methyl-1-piperidinyl; 4-amino-4-methyl-1-piperidinyl.

A further embodiment of present invention is (x) a compound of formula (I) according to (viii), wherein R² is diazaspiro[4.5]decanyl.

A further embodiment of present invention is (xi) a compound of formula (I) according to (x), wherein R² is 2,9-diazaspiro[4.5]decan-2-yl.

Another embodiment of present invention is that (xii) particular compounds of formula (I) are the following:
4-[(2R,6S)-2-methyl-6-[[4-(4-methylpiperazin-1-yl)-1-piperidyl]methyl]morpholin-4-yl]pyrazolo[1,5-a]pyridine-7-carbonitrile;
N-[1-[[(2S,6R)-4-(7-cyanopyrazolo[1,5-a]pyridin-4-yl)-6-methyl-morpholin-2-yl]methyl]-4-piperidyl]-2,2-dimethyl-propanamide;
4-[(2S,6R)-2-(3,9-diazaspiro[5.5]undecan-3-ylmethyl)-6-methyl-morpholin-4-yl]pyrazolo[1,5-a]pyridine-7-carbonitrile;
4-[(2S,6R)-2-[[4-(azepan-1-yl)-1-piperidyl]methyl]-6-methyl-morpholin-4-yl]pyrazolo[1,5-a]pyridine-7-carbonitrile;
4-[(2R,6S)-2-methyl-6-(piperazin-1-ylmethyl)morpholin-4-yl]pyrazolo[1,5-a]pyridine-7-carbonitrile;
4-[(2S,6R)-2-(2,7-diazaspiro[4.4]nonan-2-ylmethyl)-6-methyl-morpholin-4-yl]pyrazolo[1,5-a]pyridine-7-carbonitrile;
4-[(2R,6S)-2-methyl-6-[[(1-methyl-4-piperidyl)amino]methyl]morpholin-4-yl]pyrazolo[1,5-a]pyridine-7-carbonitrile;
4-[(2S,6R)-2-(2,6-diazaspiro[3.3]heptan-2-ylmethyl)-6-methyl-morpholin-4-yl]pyrazolo[1,5-a]pyridine-7-carbonitrile;
4-[(2S,6R)-2-(2,7-diazaspiro[3.4]octan-2-ylmethyl)-6-methyl-morpholin-4-yl]pyrazolo[1,5-a]pyridine-7-carbonitrile;
4-[(2S,6R)-2-(2,8-diazaspiro[3.5]nonan-2-ylmethyl)-6-methyl-morpholin-4-yl]pyrazolo[1,5-a]pyridine-7-carbonitrile;
4-[(2S,6R)-2-[(6-amino-2-azaspiro[3.3]heptan-2-yl)methyl]-6-methyl-morpholin-4-yl]pyrazolo[1,5-a]pyridine-7-carbonitrile;
4-[(2S,6R)-2-[[4-(aminomethyl)-1-piperidyl]methyl]-6-methyl-morpholin-4-yl]pyrazolo[1,5-a]pyridine-7-carbonitrile;
4-[(2S,6R)-2-[[4-(2-aminoethyl)-1-piperidyl]methyl]-6-methyl-morpholin-4-yl]pyrazolo[1,5-a]pyridine-7-carbonitrile;
4-[(2S,6R)-2-[[4-(2-aminoacetyl)piperazin-1-yl]methyl]-6-methyl-morpholin-4-yl]pyrazolo[1,5-a]pyridine-7-carbonitrile;
4-[(2R,6S)-2-methyl-6-[(4-methylpiperazin-1-yl)methyl]morpholin-4-yl]pyrazolo[1,5-a]pyridine-7-carbonitrile;
4-[(2R,6S)-2-methyl-6-[[4-(1-piperidyl)-1-piperidyl]methyl]morpholin-4-yl]pyrazolo[1,5-a]pyridine-7-carbonitrile;
4-[(2S,6R)-2-[(4-amino-1-piperidyl)methyl]-6-methyl-morpholin-4-yl]pyrazolo[1,5-a]pyridine-7-carbonitrile;
4-[(2S,6R)-2-(2,8-diazaspiro[4.5]decan-2-ylmethyl)-6-methyl-morpholin-4-yl]pyrazolo[1,5-a]pyridine-7-carbonitrile;
4-[(2S,6R)-2-(2,8-diazaspiro[4.5]decan-8-ylmethyl)-6-methyl-morpholin-4-yl]pyrazolo[1,5-a]pyridine-7-carbonitrile;
4-[(2R,6R)-2-methyl-6-[(3-oxo-2,7-diazaspiro[4.4]nonan-2-yl)methyl]morpholin-4-yl]pyrazolo[1,5-a]pyridine-7-carbonitrile;
4-[(2R,6R)-2-methyl-6-[(1-oxo-2,7-diazaspiro[4.4]nonan-2-yl)methyl]morpholin-4-yl]pyrazolo[1,5-a]pyridine-7-carbonitrile;
4-[(2S,6R)-2-(2,3,3a,4,6,6a-hexahydro-1H-pyrrolo[3,4-c]pyrrol-5-ylmethyl)-6-methyl-morpholin-4-yl]pyrazolo[1,5-a]pyridine-7-carbonitrile;
4-[(2S,6R)-2-(2,8-diazaspiro[5.5]undecan-2-ylmethyl)-6-methyl-morpholin-4-yl]pyrazolo[1,5-a]pyridine-7-carbonitrile;
4-[(2S,6R)-2-(2,7-diazaspiro[3.5]nonan-2-ylmethyl)-6-methyl-morpholin-4-yl]pyrazolo[1,5-a]pyridine-7-carbonitrile;
4-[(2S,6R)-2-(2,9-diazaspiro[4.5]decan-2-ylmethyl)-6-methyl-morpholin-4-yl]pyrazolo[1,5-a]pyridine-7-carbonitrile;
4-[(2S,6R)-2-(2,7-diazaspiro[3.5]nonan-7-ylmethyl)-6-methyl-morpholin-4-yl]pyrazolo[1,5-a]pyridine-7-carbonitrile;
4-[(2S,6R)-2-(2,9-diazaspiro[5.5]undecan-9-ylmethyl)-6-methyl-morpholin-4-yl]pyrazolo[1,5-a]pyridine-7-carbonitrile;
4-[(2R,6S)-2-methyl-6-[(3-methylpiperazin-1-yl)methyl]morpholin-4-yl]pyrazolo[1,5-a]pyridine-7-carbonitrile;
4-[(2S,6R)-2-[(4-amino-4-methyl-1-piperidyl)methyl]-6-methyl-morpholin-4-yl]pyrazolo[1,5-a]pyridine-7-carbonitrile;
4-[(2S,6R)-2-(2,5-diazabicyclo[2.2.2]octan-2-ylmethyl)-6-methyl-morpholin-4-yl]pyrazolo[1,5-a]pyridine-7-carbonitrile;
4-[(2S,6R)-2-(3,8-diazabicyclo[3.2.1]octan-3-ylmethyl)-6-methyl-morpholin-4-yl]pyrazolo[1,5-a]pyridine-7-carbonitrile;
4-[(2S,6R)-2-(2,9-diazaspiro[5.5]undecan-2-ylmethyl)-6-methyl-morpholin-4-yl]pyrazolo[1,5-a]pyridine-7-carbonitrile;
4-[(2S,6R)-2-(2,6-diazaspiro[3.5]nonan-6-ylmethyl)-6-methyl-morpholin-4-yl]pyrazolo[1,5-a]pyridine-7-carbonitrile;
4-[(2S,6R)-2-[(4-amino-3,3-difluoro-1-piperidyl)methyl]-6-methyl-morpholin-4-yl]pyrazolo[1,5-a]pyridine-7-carbonitrile;
4-[(2S,6R)-2-[(4-amino-3,3-difluoro-pyrrolidin-1-yl)methyl]-6-methyl-morpholin-4-yl]pyrazolo[1,5-a]pyridine-7-carbonitrile;
4-[(2S,6R)-2-[(8-amino-3-azabicyclo[3.2.1]octan-3-yl)methyl]-6-methyl-morpholin-4-yl]pyrazolo[1,5-a]pyridine-7-carbonitrile;
4-[(2S,6R)-2-[(3-amino-8-azabicyclo[3.2.1]octan-8-yl)methyl]-6-methyl-morpholin-4-yl]pyrazolo[1,5-a]pyridine-7-carbonitrile;
4-[(2R,6S)-2-methyl-6-[[(3R)-3-phenylpiperazin-1-yl]methyl]morpholin-4-yl]pyrazolo[1,5-a]pyridine-7-carbonitrile;
4-[(2R,6S)-2-methyl-6-[(4-piperazin-1-yl-1-piperidyl)methyl]morpholin-4-yl]pyrazolo[1,5-a]pyridine-7-carbonitrile;
4-[(2S,6R)-2-(4,7-diazaspiro[2.5]octan-7-ylmethyl)-6-methyl-morpholin-4-yl]pyrazolo[1,5-a]pyridine-7-carbonitrile;
4-[(2S,6R)-2-(1,2,3,3a,4,6,7,7a-octahydropyrrolo[3,2-c]pyridin-5-ylmethyl)-6-methyl-morpholin-4-yl]pyrazolo[1,5-a]pyridine-7-carbonitrile;
4-[(2S,6R)-2-(2,6-diazaspiro[4.5]decan-2-ylmethyl)-6-methyl-morpholin-4-yl]pyrazolo[1,5-a]pyridine-7-carbonitrile;
4-[(2S,6R)-2-(3,8-diazabicyclo[3.2.1]octan-8-ylmethyl)-6-methyl-morpholin-4-yl]pyrazolo[1,5-a]pyridine-7-carbonitrile;
4-[(2S,6R)-2-(2,7-diazaspiro[4.5]decan-7-ylmethyl)-6-methyl-morpholin-4-yl]pyrazolo[1,5-a]pyridine-7-carbonitrile;
4-[(2S,6R)-2-(1,2,3,4,4a,5,7,7a-octahydropyrrolo[3,4-b]pyridin-6-ylmethyl)-6-methyl-morpholin-4-yl]pyrazolo[1,5-a]pyridine-7-carbonitrile;
4-[(2S,6R)-2-(2,8-diazaspiro[3.6]decan-2-ylmethyl)-6-methyl-morpholin-4-yl]pyrazolo[1,5-a]pyridine-7-carbonitrile;
4-[(2S,6R)-2-(1,9-diazaspiro[5.5]undecan-9-ylmethyl)-6-methyl-morpholin-4-yl]pyrazolo[1,5-a]pyridine-7-carbonitrile;
4-[(2S,6R)-2-(1,7-diazaspiro[3.5]nonan-7-ylmethyl)-6-methyl-morpholin-4-yl]pyrazolo[1,5-a]pyridine-7-carbonitrile;
4-[(2S,6R)-2-(1,3,3a,4,5,6,7,7a-octahydropyrrolo[3,4-c]pyridin-2-ylmethyl)-6-methyl-morpholin-4-yl]pyrazolo[1,5-a]pyridine-7-carbonitrile;
4-[(2R,6S)-2-methyl-6-[(2-methylpiperazin-1-yl)methyl]morpholin-4-yl]pyrazolo[1,5-a]pyridine-7-carbonitrile;
4-[(2S,6R)-2-[(4-amino-2-methyl-1-piperidyl)methyl]-6-methyl-morpholin-4-yl]pyrazolo[1,5-a]pyridine-7-carbonitrile;
4-[(2S,6R)-2-(3,7-diazabicyclo[4.2.0]octan-7-ylmethyl)-6-methyl-morpholin-4-yl]pyrazolo[1,5-a]pyridine-7-carbonitrile;
4-[(2S,6R)-2-(1,8-diazaspiro[4.5]decan-8-ylmethyl)-6-methyl-morpholin-4-yl]pyrazolo[1,5-a]pyridine-7-carbonitrile;
4-[(2R,6S)-2-methyl-6-[(4-pyrrolidin-1-yl-1-piperidyl)methyl]morpholin-4-yl]pyrazolo[1,5-a]pyridine-7-carbonitrile;
4-[(2S,6R)-2-(1,6-diazaspiro[3.3]heptan-1-ylmethyl)-6-methyl-morpholin-4-yl]pyrazolo[1,5-a]pyridine-7-carbonitrile;
4-[(2S,6R)-2-(3,8-diazabicyclo[4.2.0]octan-8-ylmethyl)-6-methyl-morpholin-4-yl]pyrazolo[1,5-a]pyridine-7-carbonitrile;
4-[(2R,6S)-2-methyl-6-(3-oxa-7,9-diazabicyclo[3.3.1]nonan-9-ylmethyl)morpholin-4-yl]pyrazolo[1,5-a]pyridine-7-carbonitrile;
4-[(2S,6R)-2-[(7-amino-3-oxa-9-azabicyclo[3.3.1]nonan-9-yl)methyl]-6-methyl-morpholin-4-yl]pyrazolo[1,5-a]pyridine-7-carbonitrile;
4-[(2S,6R)-2-[(2-benzylpiperazin-1-yl)methyl]-6-methyl-morpholin-4-yl]pyrazolo[1,5-a]pyridine-7-carbonitrile;
4-[(2R,6S)-2-methyl-6-(l-oxa-4,9-diazaspiro[5.5]undecan-9-ylmethyl)morpholin-4-yl]pyrazolo[1,5-a]pyridine-7-carbonitrile;
4-[(2S,6R)-2-(2,3,4,4a,5,6,7,7a-octahydropyrrolo[3,4-b]pyridin-1-ylmethyl)-6-methyl-morpholin-4-yl]pyrazolo[1,5-a]pyridine-7-carbonitrile;
4-[(2R,6S)-2-methyl-6-[[cis-(3aR,6aR)-3,3a,4,5,6,6a-hexahydro-2H-pyrrolo[3,4-b]pyrrol-1-yl]methyl]morpholin-4-yl]pyrazolo[1,5-a]pyridine-7-carbonitrile;
4-[(2R,6S)-2-methyl-6-[[cis-(3aS,7aS)-1,2,3,3a,5,6,7,7a-octahydropyrrolo[3,2-b]pyridin-4-yl]methyl]morpholin-4-yl]pyrazolo[1,5-a]pyridine-7-carbonitrile;
4-[(2S,6R)-2-(3,4,4a,5,6,7,8,8a-octahydro-2H-1,5-naphthyridin-1-ylmethyl)-6-methyl-morpholin-4-yl]pyrazolo[1,5-a]pyridine-7-carbonitrile;
Endo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile;
Exo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile;
4-[(2S,6R)-2-(2,9-diazaspiro[5.5]undecan-2-ylmethyl)-6-methyl-morpholin-4-yl]-3-fluoro-pyrazolo[1,5-a]pyridine-7-carbonitrile;
3-fluoro-4-[(2S,6R)-2-(2,7-diazaspiro[4.5]decan-7-ylmethyl)-6-methyl-morpholin-4-yl]pyrazolo[1,5-a]pyridine-7-carbonitrile;
3-fluoro-4-[(2S,6R)-2-(2,9-diazaspiro[4.5]decan-2-ylmethyl)-6-methyl-morpholin-4-yl]pyrazolo[1,5-a]pyridine-7-carbonitrile;
4-[(2S,6R)-2-(3,7-diazabicyclo[4.2.0]octan-3-ylmethyl)-6-methyl-morpholin-4-yl]pyrazolo[1,5-a]pyridine-7-carbonitrile;
4-[(2R,6S)-2-methyl-6-(1-oxa-4,9-diazaspiro[5.5]undecan-4-ylmethyl)morpholin-4-yl]pyrazolo[1,5-a]pyridine-7-carbonitrile;
1-[[(2S,6R)-4-(7-cyanopyrazolo[1,5-a]pyridin-4-yl)-6-methyl-morpholin-2-yl]methyl]piperidine-3-carboxamide;
4-[(2S,6R)-2-[(4-amino-4-phenyl-1-piperidyl)methyl]-6-methyl-morpholin-4-yl]pyrazolo[1,5-a]pyridine-7-carbonitrile;
4-[(2S,6R)-2-[[(3R,4S)-4-amino-3-methyl-1-piperidyl]methyl]-6-methyl-morpholin-4-yl]pyrazolo[1,5-a]pyridine-7-carbonitrile;
4-[(2S,6R)-2-[[4-(dimethylamino)-3,3a,4,5,6,6a-hexahydro-1H-cyclopenta[c]pyrrol-2-yl]methyl]-6-methyl-morpholin-4-yl]pyrazolo[1,5-a]pyridine-7-carbonitrile;
4-[(2R,6S)-2-methyl-6-[(9-methyl-6-oxa-2,9-diazaspiro[4.5]decan-2-yl)methyl]morpholin-4-yl]pyrazolo[1,5-a]pyridine-7-carbonitrile; and
4-[(2S,6R)-2-[[2-benzyl-4-(4-piperidyl)piperazin-1-yl]methyl]-6-methyl-morpholin-4-yl]pyrazolo[1,5-a]pyridine-7-carbonitrile;

or a pharmaceutically acceptable salt, enantiomer or diastereomer thereof.

### Synthesis

The compounds of the present invention can be prepared by any conventional means. Suitable processes for synthesizing these compounds as well as their starting materials are provided in the schemes below and in the examples. All substituents, in particular, R¹ to R⁴ are as defined above unless otherwise indicated. Furthermore, and unless explicitly otherwise stated, all reactions, reaction conditions, abbreviations and symbols have the meanings well known to a person of ordinary skill in organic chemistry.

General synthetic route for preparing the compound of formula (I) or (II) is shown below.

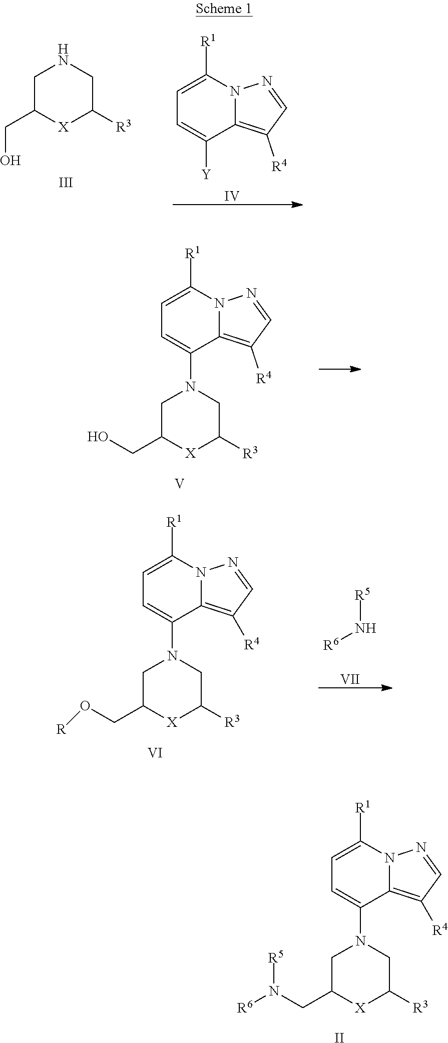

wherein R is Ms, Ts or Tf. R⁵ and R⁶ are independently selected from H and heterocyclyl, or R⁵ and R⁶ together with the nitrogen they are attached to form a heterocyclyl. Y is halogen.

The coupling of compound of formula (III) with halide (IV) can be achieved by direct coupling in the presence of a base, such as DIPEA and K₂CO₃, or under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as Ruphos Pd-G2, and a base, such as Cs₂CO₃, to provide compound of formula (V). Subsequently the hydroxy group of compound of formula (V) is converted to a leaving group, such as —OTf, —OTs, or —OMs, under basic condition, such as DIPEA, TEA, K₂CO₃ or 2,6-dimethylpyridine, with Tf₂O, TsCl or MsCl. Compound of formula (VI) was further coupled with amine (VII) in the presence of base, such as K₂CO₃, DIPEA or Cs₂CO₃, to afford compound of formula (II). In some embodiment, the coupling of compound of formula (VI) and amine (VII) may give a product containing a protecting group, e.g. Boc, originated from amine (VII), which will be removed before affording the final compound of formula (II).

This invention also relates to a process for the preparation of a compound of formula (I) comprising any of the following steps:

a) the reaction of compound of formula (VI),

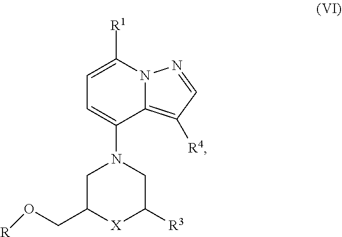

with amine (VII) in the presence of a base;

In step a) and d) the base can be for example K₂CO₃, DIPEA or Cs₂CO₃.

A compound of formula (I) or (II) when manufactured according to the above process is also an object of the invention.

Compounds of this invention can be obtained as mixtures of diastereomers or enantiomers, which can be separated by methods well known in the art, e.g. (chiral) HPLC or SFC.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN: acetonitrile
DIPEA: N,N-diisopropylethylamine
EtOAc: ethyl acetate
FA: formic acid
HATU: 1-[Bis(dimethylamino)methylene]-1H-1,2,3-triazolo[4,5-b]pyridinium 3-oxid hexafluorophosphate
IC₅₀: half inhibition concentration
IPA: isopropanol
LCMS liquid chromatography-mass spectrometry
L-DATA: Di-p-anisoyl-L-tartaric acid
MS: mass spectrometry
Ms: methylsulfonyl
NCS: N-chlorosuccinimide
NIS: N-iodosuccinimide
prep-HPLC: preparative high performance liquid chromatography
PPh₃: triphenylphosphine
rt: room temperature
RuPhos Pd G2: chloro(2-dicyclohexylphosphino-2′,6′-diisopropoxy-1,1′-biphenyl)[2-(2′-amino-1,1′-biphenyl)]palladium(II) 2nd generation
SFC: supercritical fluid chromatography
SelectFluor: 1-chloromethyl-4-fluoro-1,4-diazoniabicyclo[2.2.2]octane bis(tetrafluoroborate)
TEA: triethylamine
TEMPO: tetramethylpiperidinooxy
Tf: trifluoromethanesulfonyl
TFA: trifluoroacetic acid
THF: tetrahydrofuran
Ts p-toluenesulfonyl
v/v: volume ratio
DDI: drug-drug-interaction
LYSA: lyophilisation solubility assay
HLM: human liver microsome

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.10% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 am, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃.H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in acetonitrile;

Acidic condition II: A: 0.0375% TFA in H₂O; B: 0.01875% TFA in acetonitrile;

Basic condition I: A: 0.1% NH₃.H₂O in H₂O; B: acetonitrile;

Basic condition II: A: 0.025% NH₃.H₂O in H₂O; B: acetonitrile;

Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Example 1

### 4-[(2R,6S)-2-methyl-6-[[4-(4-methylpiperazin-1-yl)-1-piperidyl]methyl]morpholin-4-yl]pyrazolo[1,5-a]pyridine-7-carbonitrile

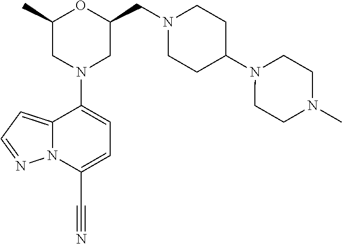 [SMILES: C[C@@H]1CN(c2ccc(C#N)n3nccc23)C[C@H](CN2CCC(N3CCN(C)CC3)CC2)O1]

The title compound was synthesized according to the following scheme:

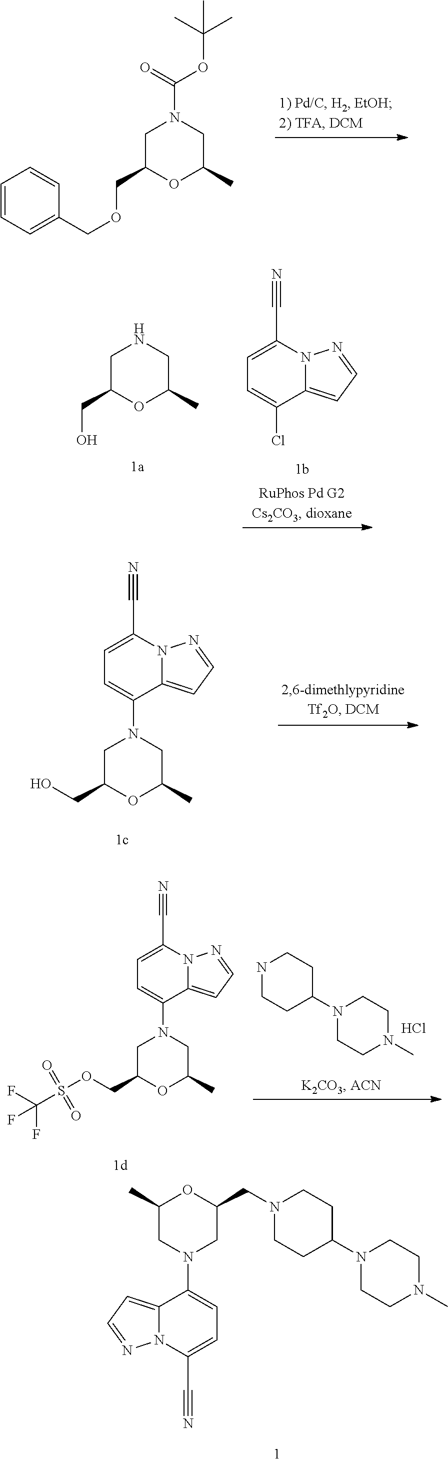

### Step 1: Preparation of [(2R,6R)-6-methylmorpholin-2-yl]methanol (Compound 1a)

To a solution of tert-butyl (2R,6R)-2-(benzyloxymethyl)-6-methyl-morpholine-4-carboxylate (Reference: US 20150105370 A1) (22.0 g, 68.4 mmol) in EtOH (500 mL) was added Pd/C (7.28 g, 10% wet) and stirred for 48 hrs at 30° C. under H2 atmosphere. The solution was then filtered, and the filtrate was concentrated to give an intermediate (15 g) as a colorless oil. To a solution of this intermediate (231 mg, 1.0 mmol) in DCM (2 mL) was added TFA (1 mL) at 0° C. The reaction mixture was stirred at rt for 2 hrs, then concentrated to give a crude compound 1a (250 mg) which was directly used in next step. MS: calc'd 132 (MH⁺), measured 132 (MHI).

### Step 2: Preparation of 4-[(2R,6R)-2-(hydroxymethyl)-6-methyl-morpholin-4-yl]pyrazolo[1,5-a]pyridine-7-carbonitrile (Compound 1c)

To a solution of crude [(2R,6R)-6-methylmorpholin-2-yl]methanol (compound 1a, 250 mg, ˜1.0 mmol), 4-chloropyrazolo[1,5-a]pyridine-7-carbonitrile (CAS: 1268520-74-6, Pharmablock) (compound 1b, 450 mg, 2.5 mmol) and Cs₂CO₃ (3.3 g, 10.1 mmol) in dioxane (5 mL) was added Ruphos Pd G2 (98 mg, 0.13 mmol). The reaction mixture was degassed and heated at 90° C. (oil bath) for 3 hrs, then cooled to rt, diluted with EtOAc (10 mL), and filtered through celite. The filtrate was concentrated to give a brown oil which was purified by column chromatography to give compound 1c (521 mg) as a yellowish oil. MS: calc'd 273 (MH⁺), measured 273 (MH⁺).

### Step 3: Preparation of [(2R,6R)-4-(7-cyanopyrazolo[1,5-a]pyridin-4-yl)-6-methyl-morpholin-2-yl]methyl trifluoromethanesulfonate (Compound 1d)

To a solution of 4-[(2R,6R)-2-(hydroxymethyl)-6-methyl-morpholin-4-yl]pyrazolo[1,5-a]pyridine-7-carbonitrile (compound 1c, 190 mg, 0.70 mmol) and 2,6-dimethylpyridine (150 mg, 1.4 mmol) in DCM (4 mL) was added trifluoromethanesulfonic anhydride (295 mg, 1.05 mmol) dropwise at 0° C. The mixture was stirred at 0° C. for 1 h. The mixture was then diluted with DCM, washed with sat. NH₄Cl and brine, dried over Na₂SO₄, and concentrated to give a crude product which was purified by column chromatography to give compound 1d (166 mg) as a white solid. MS: calc'd 405 (MH⁺), measured 405 (MH⁺).

### Step 4: Preparation of 4-[(2R,6S)-2-methyl-6-[[4-(4-methylpiperazin-1-yl)-1-piperidyl]methyl]morpholin-4-yl]pyrazolo[1,5-a]pyridine-7-carbonitrile (Example 1)

To a solution of [(2R,6R)-4-(7-cyanopyrazolo[1,5-a]pyridin-4-yl)-6-methyl-morpholin-2-yl]methyl trifluoromethanesulfonate (compound 1d, 41 mg, 0.10 mmol) and 1-methyl-4-(piperidin-4-yl)piperazine hydrochloride (33 mg, 0.15 mmol) in ACN (4 mL) was added K₂CO₃ (56 mg, 0.41 mmol) at rt. After the reaction mixture was refluxed for 6 hrs. The mixture was then diluted with ACN and filtered through celite. The filtrate was concentrated to give a yellow solid which was purified by prep-HPLC to give Example 1 (7 mg) as a white solid. MS: calc'd 438 (MH⁺), measured 438 (MH⁺). ¹H NMR (400 MHz, METHANOL-d4) δ 8.02 (d, J=2.4 Hz, 1H), 7.46 (d, J=7.9 Hz, 1H), 6.86 (d, J=2.4 Hz, 1H), 6.58 (d, J=7.9 Hz, 1H), 4.04-3.86 (m, 2H), 3.81 (br d, J=12.3 Hz, 1H), 3.77-3.68 (m, 1H), 3.16 (br d, J=11.9 Hz, 1H), 3.01 (br s, 1H), 2.77-2.58 (m, 5H), 2.58-2.36 (m, 6H), 2.28 (s, 3H), 2.28-2.17 (m, 2H), 2.17-2.02 (m, 2H), 1.90 (br d, J=10.4 Hz, 2H), 1.64-1.51 (m, 2H), 1.25 (d, J=6.2 Hz, 3H).

### Example 2

### N-[1-[[(2S,6R)-4-(7-cyanopyrazolo[1,5-a]pyridin-4-yl)-6-methyl-morpholin-2-yl]methyl]-4-piperidyl]-2,2-dimethyl-propanamide

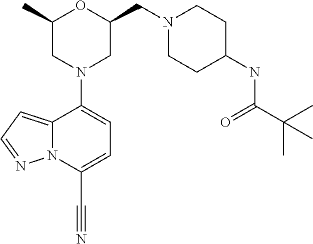 [SMILES: C[C@@H]1CN(c2ccc(C#N)n3nccc23)C[C@H](CN2CCC(NC(=O)C(C)(C)C)CC2)O1]

The title compound was prepared in analogy to the preparation of Example 1 by using 2,2-dimethyl-N-(4-piperidyl)propanamide instead of 1-methyl-4-(piperidin-4-yl)piperazine hydrochloride. Example 2 (11 mg) was obtained as a white powder. MS: calc'd 439 (MH⁺), measured 439 (MH⁺). ¹H NMR (400 MHz, METHANOL-d4) δ 8.03 (d, J=2.4 Hz, 1H), 7.47 (d, J=8.1 Hz, 1H), 6.86 (d, J=2.4 Hz, 1H), 6.59 (d, J=8.1 Hz, 1H), 4.03-3.96 (m, 1H), 3.96-3.86 (m, 1H), 3.81 (br d, J=12.3 Hz, 1H), 3.77-3.71 (m, 1H), 3.71-3.63 (m, 1H), 3.10 (br d, J=12.0 Hz, 1H), 2.98 (br d, J=11.7 Hz, 1H), 2.65 (td, J=10.1, 12.1 Hz, 2H), 2.60-2.46 (m, 2H), 2.29-2.13 (m, 2H), 1.80 (br d, J=12.2 Hz, 2H), 1.68-1.54 (m, 2H), 1.26 (d, J=6.2 Hz, 3H), 1.17 (s, 9H).

### Example 3

### 4-[(2S,6R)-2-(3,9-diazaspiro[5.5]undecan-3-ylmethyl)-6-methyl-morpholin-4-yl]pyrazolo[1,5-a]pyridine-7-carbonitrile

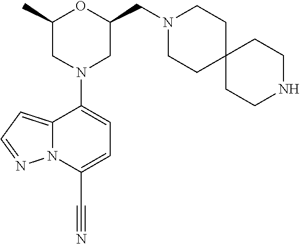 [SMILES: C[C@@H]1CN(c2ccc(C#N)n3nccc23)C[C@H](CN2CCC3(CCNCC3)CC2)O1]

To a solution of [(2R,6R)-4-(7-cyanopyrazolo[1,5-a]pyridin-4-yl)-6-methyl-morpholin-2-yl]methyl trifluoromethanesulfonate (compound 1d, 41 mg, 0.10 mmol) and tert-butyl 3,9-diazaspiro[5.5]undecane-3-carboxylate (39 mg, 0.15 mmol) in ACN (4 mL) was added K₂CO₃ (56 mg, 0.41 mmol) at rt. After the reaction mixture was refluxed for 6 hrs. The mixture was then diluted with ACN and filtered through celite. The filtrate was concentrated to give a yellow solid. The crude product was then dissolved in DCM (2 mL), treated with TFA (1 mL). The reaction mixture was stirred at rt for 1 h, then concentrated to give a yellow oil which was purified by prep-HPLC to give Example 3 (22 mg) as a light yellow solid. MS: calc'd 409 (MH⁺), measured 409 (MH⁺). ¹H NMR (400 MHz, METHANOL-d4) δ 8.02 (d, J=2.3 Hz, 1H), 7.46 (d, J=7.9 Hz, 1H), 6.88 (d, J=2.4 Hz, 1H), 6.61 (d, J=8.1 Hz, 1H), 4.32 (br t, J=10.0 Hz, 1H), 4.08-3.95 (m, 1H), 3.78 (br t, J=11.9 Hz, 2H), 3.69-3.51 (m, 2H), 3.43-3.33 (m, 2H), 3.29-3.17 (m, 6H), 2.80-2.65 (m, 2H), 2.09-1.89 (m, 4H), 1.89-1.66 (m, 4H), 1.30 (d, J=6.2 Hz, 3H).

### Example 4

### 4-[(2S,6R)-2-[[4-(azepan-1-yl)-1-piperidyl]methyl]-6-methyl-morpholin-4-yl]pyrazolo[1,5-a]pyridine-7-carbonitrile

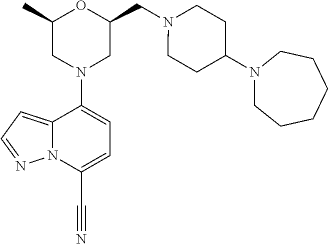 [SMILES: C[C@@H]1CN(c2ccc(C#N)n3nccc23)C[C@H](CN2CCC(N3CCCCCC3)CC2)O1]

The title compound was prepared in analogy to the preparation of Example 1 by using 1-(4-piperidyl)azepane hydrochloride instead of 1-methyl-4-(piperidin-4-yl)piperazine hydrochloride. Example 4 (13 mg) was obtained as a white powder. MS: calc'd 437 (MH⁺), measured 437 (MH⁺). ¹H NMR (400 MHz, METHANOL-d4) δ 8.04 (d, J=2.3 Hz, 1H), 7.47 (d, J=7.9 Hz, 1H), 6.88 (d, J=2.4 Hz, 1H), 6.60 (d, J=7.9 Hz, 1H), 4.14-4.05 (m, 1H), 4.01-3.91 (m, 1H), 3.79 (br t, J=14.1 Hz, 2H), 3.44-3.34 (m, 7H), 2.77 (br d, J=5.6 Hz, 2H), 2.75-2.69 (m, 1H), 2.69-2.62 (m, 1H), 2.49 (q, J=12.6 Hz, 2H), 2.21-2.11 (m, 2H), 2.03-1.86 (m, 6H), 1.81-1.70 (m, 4H), 1.28 (d, J=6.1 Hz, 3H).

### Example 5

### 4-[(2R,6S)-2-methyl-6-(piperazin-1-ylmethyl)morpholin-4-yl]pyrazolo[1,5-a]pyridine-7-carbonitrile

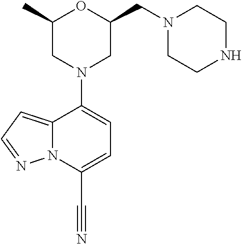 [SMILES: C[C@@H]1CN(c2ccc(C#N)n3nccc23)C[C@H](CN2CCNCC2)O1]

The title compound was prepared in analogy to the preparation of Example 3 by using tert-butyl piperazine-1-carboxylate instead of tert-butyl 3,9-diazaspiro[5.5]undecane-3-carboxylate. Example 5 (21 mg) was obtained as a white powder. MS: calc'd 341 (MH⁺), measured 341 (MH⁺). ¹H NMR (400 MHz, METHANOL-d4) δ 8.02 (d, J=2.4 Hz, 1H), 7.46 (d, J=8.1 Hz, 1H), 6.85 (d, J=2.4 Hz, 1H), 6.58 (d, J=8.1 Hz, 1H), 4.06-3.96 (m, 1H), 3.95-3.85 (m, 1H), 3.81 (br d, J=12.3 Hz, 1H), 3.74 (dd, J=2.0, 12.3 Hz, 1H), 2.92 (t, J=4.9 Hz, 4H), 2.73-2.60 (m, 4H), 2.59-2.47 (m, 4H), 1.25 (d, J=6.2 Hz, 3H).

### Example 6

### 4-[(2S,6R)-2-(2,7-diazaspiro[4.4]nonan-2-ylmethyl)-6-methyl-morpholin-4-yl]pyrazolo[1,5-a]pyridine-7-carbonitrile

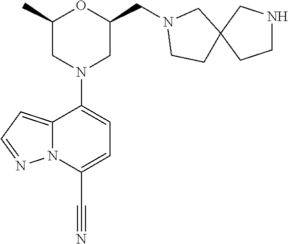 [SMILES: C[C@@H]1CN(c2ccc(C#N)n3nccc23)C[C@H](CN2CCC3(CCNC3)C2)O1]

The title compound was prepared in analogy to the preparation of Example 3 by using tert-butyl 2,7-diazaspiro[4.4]nonane-2-carboxylate instead of tert-butyl 3,9-diazaspiro[5.5]undecane-3-carboxylate. Example 6 (27 mg) was obtained as a light yellow oil. MS: calc'd 381 (MH⁺), measured 381 (MH⁺). ¹H NMR (400 MHz, METHANOL-d4) δ 8.02 (d, J=2.2 Hz, 1H), 7.45 (d, J=7.9 Hz, 1H), 6.89-6.84 (m, 1H), 6.59 (d, J=7.9 Hz, 1H), 4.16-4.07 (m, 1H), 4.02-3.91 (m, 1H), 3.78 (br dd, J=12.6, 18.6 Hz, 2H), 3.39 (br t, J=7.2 Hz, 2H), 3.36-3.31 (m, 2H), 3.30-3.19 (m, 3H), 3.19-3.09 (m, 2H), 3.09-2.99 (m, 1H), 2.77-2.61 (m, J=11.0, 11.0, 17.8 Hz, 2H), 2.21-2.01 (m, 4H), 1.29 (d, J=6.2 Hz, 3H).

### Example 7

### 4-[(2R,6S)-2-methyl-6-[[(1-methyl-4-piperidyl)amino]methyl]morpholin-4-yl]pyrazolo[1,5-a]pyridine-7-carbonitrile

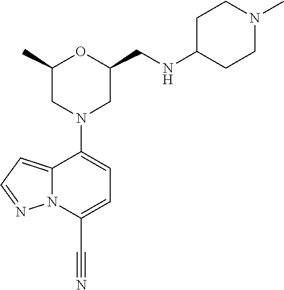 [SMILES: C[C@@H]1CN(c2ccc(C#N)n3nccc23)C[C@H](CNC2CCN(C)CC2)O1]

The title compound was prepared in analogy to the preparation of Example 1 by using 1-methylpiperidin-4-amine hydrochloride instead of 1-methyl-4-(piperidin-4-yl)piperazine hydrochloride. Example 7 (22 mg) was obtained as a light brown solid. MS: calc'd 369 (MH⁺), measured 369 (MH⁺). ¹H NMR (400 MHz, METHANOL-d4) δ 8.03 (d, J=2.1 Hz, 1H), 7.51-7.43 (m, 1H), 6.93-6.86 (m, 1H), 6.60 (d, J=7.9 Hz, 1H), 4.61-4.45 (m, 1H), 4.13-4.02 (m, 1H), 4.02-3.93 (m, 1H), 3.92-3.70 (m, 3H), 3.70-3.54 (m, 1H), 3.54-3.44 (m, 1H), 3.43-3.33 (m, 2H), 3.22-3.11 (m, 1H), 3.08-2.98 (m, 1H), 2.88-2.73 (m, 2H), 2.73-2.65 (m, 3H), 2.30-2.15 (m, 2H), 1.88-1.71 (m, 1H), 1.35-1.24 (m, 3H).

### Example 8

### 4-[(2S,6R)-2-(2,6-diazaspiro[3.3]heptan-2-ylmethyl)-6-methyl-morpholin-4-yl]pyrazolo[1,5-a]pyridine-7-carbonitrile

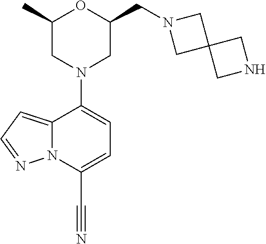 [SMILES: C[C@@H]1CN(c2ccc(C#N)n3nccc23)C[C@H](CN2CC3(CNC3)C2)O1]

The title compound was prepared in analogy to the preparation of Example 3 by using tert-butyl 2,6-diazaspiro[3.3]heptane-2-carboxylate instead of tert-butyl 3,9-diazaspiro[5.5]undecane-3-carboxylate. Example 8 (14 mg) was obtained as a white powder. MS: calc'd 353 (MH⁺), measured 353 (MH⁺). ¹H NMR (400 MHz, METHANOL-d4) δ 8.05 (d, J=2.3 Hz, 1H), 7.49 (d, J=7.9 Hz, 1H), 6.88 (d, J=2.4 Hz, 1H), 6.60 (d, J=7.9 Hz, 1H), 3.97-3.78 (m, 6H), 3.79-3.67 (m, 2H), 3.53-3.45 (m, 4H), 2.72-2.57 (m, 4H), 1.26 (d, J=6.2 Hz, 3H).

### Example 9

### 4-[(2S,6R)-2-(2,7-diazaspiro[3.4]octan-2-ylmethyl)-6-methyl-morpholin-4-yl]pyrazolo[1,5-a]pyridine-7-carbonitrile

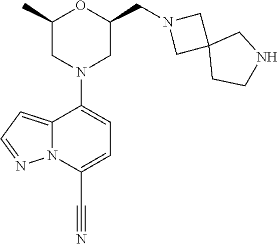 [SMILES: C[C@@H]1CN(c2ccc(C#N)n3nccc23)C[C@H](CN2CC3(CCNC3)C2)O1]

The title compound was prepared in analogy to the preparation of Example 3 by using tert-butyl 2,6-diazaspiro[3.4]octane-6-carboxylate (CAS: 885270-86-0, PharmaBlock) instead of tert-butyl 3,9-diazaspiro[5.5]undecane-3-carboxylate. Example 9 (18 mg) was obtained as a light yellow powder. MS: calc'd 367 (MH⁺), measured 367 (MH⁺). ¹H NMR (400 MHz, METHANOL-d4) δ 8.04 (d, J=2.3 Hz, 1H), 7.48 (d, J=7.9 Hz, 1H), 6.88 (d, J=2.4 Hz, 1H), 6.62 (d, J=8.1 Hz, 1H), 4.47-4.30 (m, 4H), 4.19-4.08 (m, 1H), 4.02-3.91 (m, 1H), 3.76 (br d, J=12.0 Hz, 2H), 3.61 (br s, 2H), 3.56-3.50 (m, 1H), 3.48-3.41 (m, 1H), 3.41-3.34 (m, 1H), 3.41-3.34 (m, 1H), 2.78-2.63 (m, 2H), 2.51-2.38 (m, 2H), 1.29 (d, J=6.2 Hz, 3H).

### Example 10

### 4-[(2S,6R)-2-(2,8-diazaspiro[3.5]nonan-2-ylmethyl)-6-methyl-morpholin-4-yl]pyrazolo[1,5-a]pyridine-7-carbonitrile

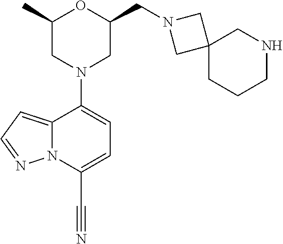 [SMILES: C[C@@H]1CN(c2ccc(C#N)n3nccc23)C[C@H](CN2CC3(CCCNC3)C2)O1]

The title compound was prepared in analogy to the preparation of Example 3 by using tert-butyl 2,6-diazaspiro[3.5]nonane-6-carboxylate (CAS: 885272-17-3, PharmaBlock) instead of tert-butyl 3,9-diazaspiro[5.5]undecane-3-carboxylate. Example 10 (25 mg) was obtained as a light yellow solid. MS: calc'd 381 (MH⁺), measured 381 (MH⁺). ¹H NMR (400 MHz, METHANOL-d4) δ 8.04 (d, J=2.4 Hz, 1H), 7.48 (d, J=7.9 Hz, 1H), 6.89 (d, J=2.4 Hz, 1H), 6.62 (d, J=8.1 Hz, 1H), 4.45-4.04 (m, 5H), 4.03-3.89 (m, 1H), 3.76 (br d, J=12.8 Hz, 2H), 3.58-3.39 (m, 4H), 3.14 (t, J=5.6 Hz, 2H), 2.78-2.63 (m, 2H), 2.08 (br s, 2H), 1.87 (br s, 2H), 1.29 (d, J=6.2 Hz, 3H).

### Example 11

### 4-[(2S,6R)-2-[(6-amino-2-azaspiro[3.3]heptan-2-yl)methyl]-6-methyl-morpholin-4-yl]pyrazolo[1,5-a]pyridine-7-carbonitrile

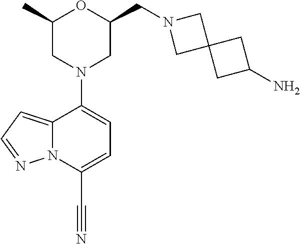 [SMILES: C[C@@H]1CN(c2ccc(C#N)n3nccc23)C[C@H](CN2CC3(CC(N)C3)C2)O1]

The title compound was prepared in analogy to the preparation of Example 3 by using tert-butyl N-(2-azaspiro[3.3]heptan-6-yl)carbamate (CAS: 1118786-85-8, PharmaBlock) instead of tert-butyl 3,9-diazaspiro[5.5]undecane-3-carboxylate. Example 11 (24 mg) was obtained as a light yellow solid. MS: calc'd 367 (MH⁺), measured 367 (MH⁺). ¹H NMR (400 MHz, METHANOL-d4) δ 8.04 (d, J=2.4 Hz, 1H), 7.48 (d, J=7.9 Hz, 1H), 6.88 (d, J=2.4 Hz, 1H), 6.61 (d, J=8.1 Hz, 1H), 4.50-4.20 (m, 4H), 4.13-4.04 (m, 1H), 4.00-3.90 (m, 1H), 3.81-3.69 (m, 3H), 3.47-3.41 (m, 1H), 3.38-3.33 (m, 1H), 2.85 (br s, 1H), 2.69 (ddd, J=10.5, 12.5, 16.6 Hz, 3H), 2.47 (br s, 2H), 1.29 (d, J=6.2 Hz, 3H).

### Example 12

### 4-[(2S,6R)-2-[[4-(aminomethyl)-1-piperidyl]methyl]-6-methyl-morpholin-4-yl]pyrazolo[1,5-a]pyridine-7-carbonitrile

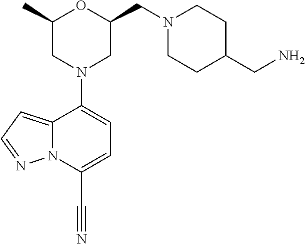 [SMILES: C[C@@H]1CN(c2ccc(C#N)n3nccc23)C[C@H](CN2CCC(CN)CC2)O1]

The title compound was prepared in analogy to the preparation of Example 3 by using tert-butyl N-(4-piperidylmethyl)carbamate instead of tert-butyl 3,9-diazaspiro[5.5]undecane-3-carboxylate. Example 12 (10 mg) was obtained as a light brown solid. MS: calc'd 369 (MH⁺), measured 369 (MH⁺). ¹H NMR (400 MHz, METHANOL-d4) δ 8.04 (d, J=2.4 Hz, 1H), 7.49 (d, J=7.9 Hz, 1H), 6.90 (d, J=2.4 Hz, 1H), 6.63 (d, J=8.1 Hz, 1H), 4.33 (br t, J=9.0 Hz, 1H), 4.07-3.97 (m, 1H), 3.86-3.70 (m, 4H), 3.40-3.32 (m, 1H), 3.30-3.22 (m, 1H), 3.18-3.07 (m, J=6.5 Hz, 2H), 2.93 (br d, J=6.5 Hz, 2H), 2.79-2.67 (m, 2H), 2.16-1.94 (m, 3H), 1.76-1.55 (m, 2H), 1.31 (d, J=6.2 Hz, 3H).

### Example 13

### 4-[(2S,6R)-2-[[4-(2-aminoethyl)-1-piperidyl]methyl]-6-methyl-morpholin-4-yl]pyrazolo[1,5-a]pyridine-7-carbonitrile

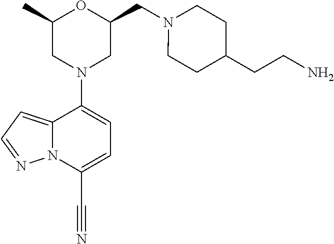 [SMILES: C[C@@H]1CN(c2ccc(C#N)n3nccc23)C[C@H](CN2CCC(CCN)CC2)O1]

The title compound was prepared in analogy to the preparation of Example 3 by using tert-butyl N-[2-(4-piperidyl)ethyl]carbamate instead of tert-butyl 3,9-diazaspiro[5.5]undecane-3-carboxylate. Example 13 (15 mg) was obtained as a light brown solid. MS: calc'd 383 (MH⁺), measured 383 (MH⁺). ¹H NMR (400 MHz, METHANOL-d4) δ 8.04 (d, J=2.4 Hz, 1H), 7.48 (d, J=7.9 Hz, 1H), 6.89 (d, J=2.4 Hz, 1H), 6.62 (d, J=7.9 Hz, 1H), 4.32 (br t, J=9.8 Hz, 1H), 4.06-3.97 (m, 1H), 3.85-3.66 (m, 4H), 3.45-3.32 (m, 1H), 3.29-3.23 (m, 1H), 3.12-2.95 (m, 4H), 2.79-2.66 (m, 2H), 2.09-1.93 (m, 2H), 1.73 (br s, 1H), 1.71-1.59 (m, 3H), 1.59-1.49 (m, 1H), 1.31 (d, J=6.4 Hz, 3H).

### Example 14

### 4-[(2S,6R)-2-[[4-(2-aminoacetyl)piperazin-1-yl]methyl]-6-methyl-morpholin-4-yl]pyrazolo[1,5-a]pyridine-7-carbonitrile

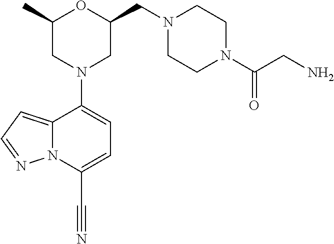 [SMILES: C[C@@H]1CN(c2ccc(C#N)n3nccc23)C[C@H](CN2CCN(C(=O)CN)CC2)O1]

The title compound was prepared according to the following scheme:

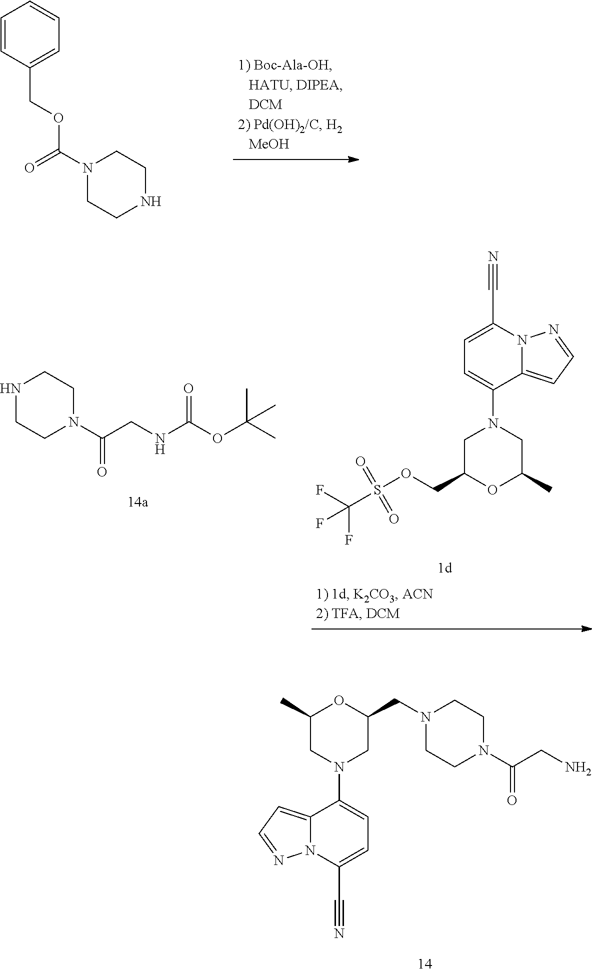 [SMILES: O=C(OCc1ccccc1)N1CCNCC1 ; CC(C)(C)OC(=O)NCC(=O)N1CCNCC1 ; C[C@@H]1CN(c2ccc(C#N)n3nccc23)C[C@H](COS(=O)(=O)C(F)(F)F)O1 ; C[C@@H]1CN(c2ccc(C#N)n3nccc23)C[C@H](CN2CCN(C(=O)CN)CC2)O1]

### Step 1: Preparation of tert-butyl N-(2-oxo-2-piperazin-1-yl-ethyl)carbamate

To a solution of Boc-Ala-OH (398 mg, 2.3 mmol), benzyl piperazine-1-carboxylate (500 mg, 2.3 mmol) and DIPEA (587 mg, 0.79 mL, 4.5 mmol) in DCM (10 mL) was added HATU (1.29 g, 3.4 mmol). The reaction mixture was stirred at rt overnight, then diluted with DCM, washed with sat. NH₄Cl and brine, dried over Na₂SO₄, and concentrated to give an oil which was purified by column chromatography to give a product (900 mg) as a colorless oil. To the solution of above product (200 mg, 0.53 mmol) in MeOH (20 mL) was added Pd(OH)₂ (20% on carbon, wet, 30 mg). The reaction mixture was charged with H₂ balloon and stirred at rt for 2 hrs, then filtered through celite, and the filtrated was concentrated to give compound 14a (100 mg) as an oil. MS: calc'd 244 (MH⁺), measured 244 (MH⁺).

### Step 2: Preparation of 4-[(2S,6R)-2-[[4-(2-aminoacetyl)piperazin-1-yl]methyl]-6-methyl-morpholin-4-yl]pyrazolo[1,5-a]pyridine-7-carbonitrile

The title compound was prepared in analogy to the preparation of Example 3 by using tert-butyl N-(2-oxo-2-piperazin-1-yl-ethyl)carbamate (compound 14a) instead of tert-butyl 3,9-diazaspiro[5.5]undecane-3-carboxylate. Example 14 (14 mg) was obtained as a light yellow powder. MS: calc'd 398 (MH⁺), measured 398 (MH⁺). ¹H NMR (400 MHz, METHANOL-d4) δ 8.04 (d, J=2.4 Hz, 1H), 7.49 (d, J=7.9 Hz, 1H), 6.90 (d, J=2.4 Hz, 1H), 6.63 (d, J=8.1 Hz, 1H), 4.34 (br t, J=9.8 Hz, 1H), 4.10-3.97 (m, 3H), 3.88-3.70 (m, J=13.6, 13.6 Hz, 5H), 3.60-3.32 (m, 7H), 2.79-2.67 (m, 2H), 1.32 (d, J=6.2 Hz, 3H).

### Example 15

### 4-[(2R,6S)-2-methyl-6-[(4-methylpiperazin-1-yl)methyl]morpholin-4-yl]pyrazolo[1,5-a]pyridine-7-carbonitrile

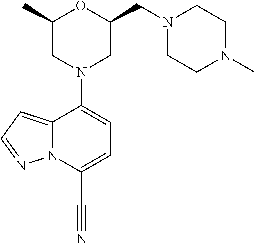 [SMILES: C[C@@H]1CN(c2ccc(C#N)n3nccc23)C[C@H](CN2CCN(C)CC2)O1]

The title compound was prepared in analogy to the preparation of Example 1 by using 1-methylpiperazine instead of 1-methyl-4-(piperidin-4-yl)piperazine hydrochloride. Example 15 (18 mg) was obtained as a light brown solid. MS: calc'd 355 (MH⁺), measured 355 (MH⁺). ¹H NMR (400 MHz, METHANOL-d4) δ 8.03 (d, J=2.4 Hz, 1H), 7.46 (d, J=8.1 Hz, 1H), 6.87 (d, J=2.4 Hz, 1H), 6.60 (d, J=7.9 Hz, 1H), 4.18-4.09 (m, 1H), 4.00-3.91 (m, 1H), 3.83-3.77 (m, 1H), 3.75 (dd, J=2.0, 12.3 Hz, 1H), 3.42 (br s, 4H), 3.29-3.07 (m, 4H), 2.99-2.94 (m, 2H), 2.91 (s, 3H), 2.74 (dd, J=10.6, 12.2 Hz, 1H), 2.67 (dd, J=10.5, 12.4 Hz, 1H), 1.27 (d, J=6.2 Hz, 3H).

### Example 16

### 4-[(2R,6S)-2-methyl-6-[[4-(1-piperidyl)-1-piperidyl]methyl]morpholin-4-yl]pyrazolo[1,5-a]pyridine-7-carbonitrile

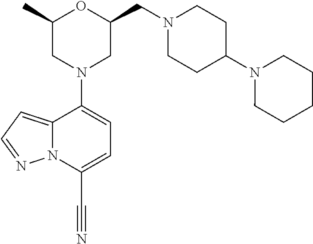 [SMILES: C[C@@H]1CN(c2ccc(C#N)n3nccc23)C[C@H](CN2CCC(N3CCCCC3)CC2)O1]

The title compound was prepared in analogy to the preparation of Example 1 by using 1-(4-piperidyl)piperidine instead of 1-methyl-4-(piperidin-4-yl)piperazine hydrochloride. Example 16 (15 mg) was obtained as a light brown solid. MS: calc'd 423 (MH⁺), measured 423 (MH⁺). ¹H NMR (400 MHz, METHANOL-d4) δ 8.04-7.99 (m, 1H), 7.49-7.44 (m, 1H), 6.91-6.86 (m, 1H), 6.64-6.59 (m, 1H), 4.33 (brt, J=10.0 Hz, 1H), 4.07-3.97 (m, 1H), 3.96-3.89 (m, 1H), 3.88-3.78 (m, 2H), 3.75 (br d, J=12.3 Hz, 1H), 3.64-3.50 (m, 3H), 3.48-3.37 (m, 1H), 3.36-3.32 (m, J=9.9 Hz, 1H), 3.28-3.16 (m, 2H), 3.06 (br t, J=11.8 Hz, 2H), 2.72 (td, J=10.1, 12.1 Hz, 2H), 2.47-2.34 (m, 2H), 2.30-2.11 (m, 2H), 2.07-1.74 (m, 5H), 1.62-1.46 (m, 1H), 1.31 (d, J=6.2 Hz, 3H).

### Example 17

### 4-[(2S,6R)-2-[(4-amino-1-piperidyl)methyl]-6-methyl-morpholin-4-yl]pyrazolo[1,5-a]pyridine-7-carbonitrile

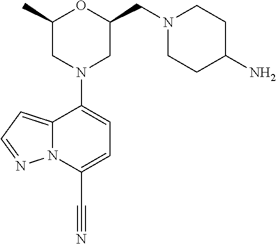 [SMILES: C[C@@H]1CN(c2ccc(C#N)n3nccc23)C[C@H](CN2CCC(N)CC2)O1]

The title compound was prepared in analogy to the preparation of Example 3 by using tert-butyl N-(4-piperidyl)carbamate instead of tert-butyl 3,9-diazaspiro[5.5]undecane-3-carboxylate. Example 17 (6 mg) was obtained as a light brown solid. MS: calc'd 355 (MH⁺), measured 355 (MH⁺). ¹H NMR (400 MHz, METHANOL-d4) δ 8.04 (d, J=2.3 Hz, 1H), 7.48 (d, J=7.9 Hz, 1H), 6.89 (d, J=2.4 Hz, 1H), 6.62 (d, J=8.1 Hz, 1H), 4.32 (br t, J=10.0 Hz, 1H), 4.07-3.96 (m, 1H), 3.95-3.64 (m, J=12.9, 12.9 Hz, 4H), 3.49 (br t, J=11.3 Hz, 1H), 3.45-3.32 (m, 2H), 3.29-3.15 (m, 2H), 2.78-2.65 (m, 2H), 2.35-2.22 (m, 2H), 2.16-1.95 (m, 2H), 1.31 (d, J=6.2 Hz, 3H).

### Example 18

### 4-[(2S,6R)-2-(2,8-diazaspiro[4.5]decan-2-ylmethyl)-6-methyl-morpholin-4-yl]pyrazolo[1,5-a]pyridine-7-carbonitrile

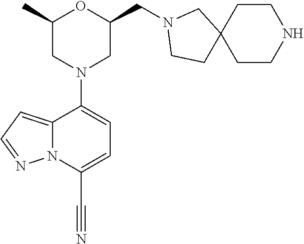 [SMILES: C[C@@H]1CN(c2ccc(C#N)n3nccc23)C[C@H](CN2CCC3(CCNCC3)C2)O1]

The title compound was prepared in analogy to the preparation of Example 3 by using tert-butyl 2,8-diazaspiro[4.5]decane-8-carboxylate (CAS: 236406-39-6, PharmaBlock) instead of tert-butyl 3,9-diazaspiro[5.5]undecane-3-carboxylate. Example 18 (7 mg) was obtained as a light brown solid. MS: calc'd 395 (MH⁺), measured 395 (MH⁺). ¹H NMR (400 MHz, METHANOL-d4) δ 8.03 (d, J=2.2 Hz, 1H), 7.47 (d, J=7.9 Hz, 1H), 6.87 (d, J=2.3 Hz, 1H), 6.60 (d, J=8.1 Hz, 1H), 4.16-4.07 (m, 1H), 4.02-3.92 (m, 1H), 3.81 (br d, J=12.1 Hz, 1H), 3.75 (br d, J=12.2 Hz, 1H), 3.27-3.15 (m, 6H), 3.15-2.94 (m, 4H), 2.75-2.61 (m, 2H), 1.95 (br t, J=7.0 Hz, 2H), 1.88 (br s, 4H), 1.29 (d, J=6.2 Hz, 3H).

### Example 19

### 4-[(2S,6R)-2-(2,8-diazaspiro[4.5]decan-8-ylmethyl)-6-methyl-morpholin-4-yl]pyrazolo[1,5-a]pyridine-7-carbonitrile

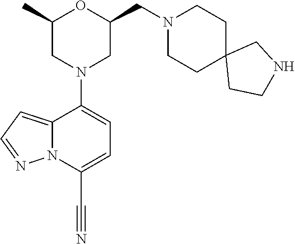 [SMILES: C[C@@H]1CN(c2ccc(C#N)n3nccc23)C[C@H](CN2CCC3(CCNC3)CC2)O1]

The title compound was prepared in analogy to the preparation of Example 3 by using tert-butyl 2,8-diazaspiro[4.5]decane-2-carboxylate (CAS: 1180509-95-8, PharmaBlock) instead of tert-butyl 3,9-diazaspiro[5.5]undecane-3-carboxylate. Example 19 (9 mg) was obtained as a light brown solid. MS: calc'd 395 (MH⁺), measured 395 (MH⁺). ¹H NMR (400 MHz, METHANOL-d4) δ 8.03 (d, J=2.4 Hz, 1H), 7.47 (d, J=7.9 Hz, 1H), 6.89 (d, J=2.4 Hz, 1H), 6.62 (d, J=8.1 Hz, 1H), 4.33 (br t, J=10.0 Hz, 1H), 4.10-3.97 (m, 1H), 3.84-3.58 (m, 4H), 3.49-3.32 (m, 5H), 3.26-3.11 (m, 3H), 2.79-2.73 (m, 1H), 2.72-2.65 (m, 1H), 2.17-1.90 (m, 6H), 1.31 (d, J=6.4 Hz, 3H).

### Example 20

### 4-[(2R,6R)-2-methyl-6-[(3-oxo-2,7-diazaspiro[4.4]nonan-2-yl)methyl]morpholin-4-yl]pyrazolo[1,5-a]pyridine-7-carbonitrile

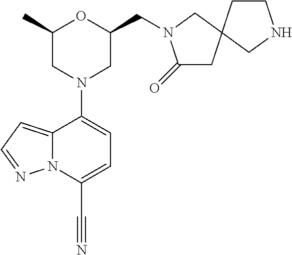 [SMILES: C[C@@H]1CN(c2ccc(C#N)n3nccc23)C[C@H](CN2CC3(CCNC3)CC2=O)O1]

The title compound was prepared in analogy to the preparation of Example 3 by using tert-butyl 8-oxo-2,7-diazaspiro[4.4]nonane-2-carboxylate (CAS: 1251009-03-6, PharmaBlock) instead of tert-butyl 3,9-diazaspiro[5.5]undecane-3-carboxylate. Example 20 (6 mg) was obtained as a light yellow solid. MS: calc'd 395 (MH⁺), measured 395 (MH⁺). ¹H NMR (400 MHz, METHANOL-d4) δ 8.02 (d, J=2.4 Hz, 1H), 7.45 (d, J=7.9 Hz, 1H), 6.87 (d, J=2.3 Hz, 1H), 6.58 (d, J=7.9 Hz, 1H), 4.06-3.98 (m, 1H), 3.95-3.86 (m, 1H), 3.79-3.70 (m, 2H), 3.69-3.63 (m, 2H), 3.57-3.39 (m, 4H), 3.34 (s, 2H), 2.74-2.62 (m, 2H), 2.62-2.48 (m, 2H), 2.23-2.07 (m, 2H), 1.26 (d, J=6.2 Hz, 3H).

### Example 21

### 4-[(2R,6R)-2-methyl-6-[(1-oxo-2,7-diazaspiro[4.4]nonan-2-yl)methyl]morpholin-4-yl]pyrazolo[1,5-a]pyridine-7-carbonitrile

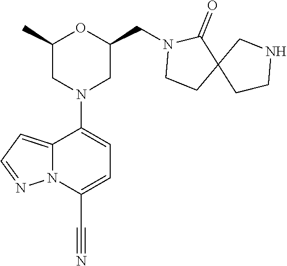 [SMILES: C[C@@H]1CN(c2ccc(C#N)n3nccc23)C[C@H](CN2CCC3(CCNC3)C2=O)O1]

The title compound was prepared in analogy to the preparation of Example 3 by using tert-butyl 1-oxo-2,7-diazaspiro[4.4]nonane-7-carboxylate (CAS: 1194376-44-7, PharmaBlock) instead of tert-butyl 3,9-diazaspiro[5.5]undecane-3-carboxylate. Example 21 (4 mg) was obtained as a yellow powder. MS: calc'd 395 (MH⁺), measured 395 (MH⁺). ¹H NMR (400 MHz, METHANOL-d4) δ 8.03 (d, J=2.3 Hz, 1H), 7.47 (d, J=7.9 Hz, 1H), 6.88 (d, J=2.4 Hz, 1H), 6.60 (d, J=8.1 Hz, 1H), 4.07-3.99 (m, 1H), 3.95-3.85 (m, 1H), 3.78 (br d, J=12.2 Hz, 1H), 3.75-3.58 (m, 3H), 3.57-3.44 (m, 4H), 3.44-3.37 (m, 1H), 3.25 (dd, J=2.0, 12.0 Hz, 1H), 2.73-2.61 (m, 2H), 2.29-2.18 (m, 3H), 2.18-2.06 (m, 1H), 1.25 (dd, J=2.9, 6.2 Hz, 3H).

### Example 22

### 4-[(2S,6R)-2-(2,3,3a,4,6,6a-hexahydro-1H-pyrrolo[3,4-c]pyrrol-5-ylmethyl)-6-methyl-morpholin-4-yl]pyrazolo[1,5-a]pyridine-7-carbonitrile

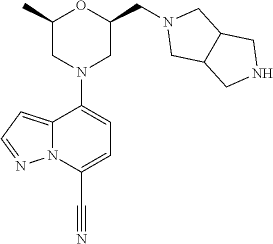 [SMILES: C[C@@H]1CN(c2ccc(C#N)n3nccc23)C[C@H](CN2CC3CNCC3C2)O1]

The title compound was prepared in analogy to the preparation of Example 3 by using tert-butyl 2,3,3a,4,6,6a-hexahydro-1H-pyrrolo[3,4-c]pyrrole-5-carboxylate (CAS: 141449-85-6, PharmaBlock) instead of tert-butyl 3,9-diazaspiro[5.5]undecane-3-carboxylate. Example 22 (12 mg) was obtained as a yellow solid. MS: calc'd 367 (MH⁺), measured 367 (MH⁺). ¹H NMR (400 MHz, METHANOL-d4) δ 8.06 (d, J=2.4 Hz, 1H), 7.50 (d, J=7.9 Hz, 1H), 6.91 (d, J=2.4 Hz, 1H), 6.65 (d, J=8.1 Hz, 1H), 4.25 (br t, J=10.0 Hz, 1H), 4.09-3.98 (m, 1H), 3.97-3.68 (m, 4H), 3.61-3.35 (m, 10H), 2.78-2.68 (m, 2H), 1.33 (d, J=6.2 Hz, 3H).

### Example 23

### 4-[(2S,6R)-2-(2,8-diazaspiro[5.5]undecan-2-ylmethyl)-6-methyl-morpholin-4-yl]pyrazolo[1,5-a]pyridine-7-carbonitrile

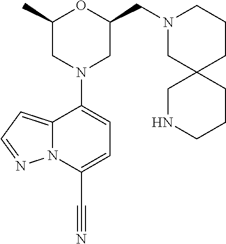 [SMILES: C[C@@H]1CN(c2ccc(C#N)n3nccc23)C[C@H](CN2CCCC3(CCCNC3)C2)O1]

The title compound was prepared in analogy to the preparation of Example 3 by using tert-butyl 2,8-diazaspiro[5.5]undecane-2-carboxylate (CAS: 189333-03-7, PharmaBlock) instead of tert-butyl 3,9-diazaspiro[5.5]undecane-3-carboxylate. Example 23 (8 mg) was obtained as a light brown solid. MS: calc'd 409 (MH⁺), measured 409 (MH⁺). ¹H NMR (400 MHz, METHANOL-d4) δ 8.06-8.01 (m, 1H), 7.48 (d, J=7.9 Hz, 1H), 6.92-6.86 (m, 1H), 6.66-6.60 (m, 1H), 4.36 (br t, J=10.0 Hz, 1H), 4.13-3.98 (m, 1H), 3.89-3.69 (m, 3H), 3.56 (br s, 1H), 3.42-3.32 (m, 2H), 3.30-3.21 (m, J=13.6 Hz, 2H), 3.21-2.85 (m, 4H), 2.80-2.68 (m, 2H), 2.21-1.77 (m, 6H), 1.76-1.40 (m, 2H), 1.36-1.27 (m, 3H).

### Example 24

### 4-[(2S,6R)-2-(2,7-diazaspiro[3.5]nonan-2-ylmethyl)-6-methyl-morpholin-4-yl]pyrazolo[1,5-a]pyridine-7-carbonitrile

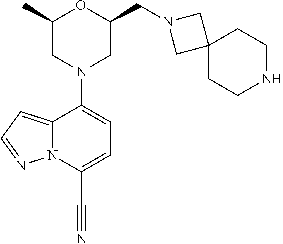 [SMILES: C[C@@H]1CN(c2ccc(C#N)n3nccc23)C[C@H](CN2CC3(CCNCC3)C2)O1]

The title compound was prepared in analogy to the preparation of Example 3 by using tert-butyl 2,7-diazaspiro[3.5]nonane-7-carboxylate (CAS: 896464-16-7, PharmaBlock) instead of tert-butyl 3,9-diazaspiro[5.5]undecane-3-carboxylate. Example 24 (13 mg) was obtained as a light brown solid. MS: calc'd 381 (MH⁺), measured 381 (MH⁺). ¹H NMR (400 MHz, METHANOL-d4) δ 8.03 (d, J=2.4 Hz, 1H), 7.47 (d, J=7.9 Hz, 1H), 6.88 (d, J=2.4 Hz, 1H), 6.61 (d, J=8.1 Hz, 1H), 4.36-4.05 (m, 5H), 4.01-3.91 (m, 1H), 3.76 (tdd, J=2.0, 4.1, 10.4 Hz, 2H), 3.58-3.51 (m, 1H), 3.48-3.40 (m, 1H), 3.22 (br s, 4H), 2.77-2.64 (m, 2H), 2.16 (br s, 4H), 1.30 (d, J=6.2 Hz, 3H).

### Example 25

### 4-[(2S,6R)-2-(2,9-diazaspiro[4.5]decan-2-ylmethyl)-6-methyl-morpholin-4-yl]pyrazolo[1,5-a]pyridine-7-carbonitrile

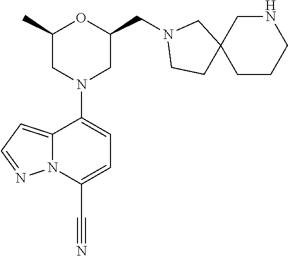 [SMILES: C[C@@H]1CN(c2ccc(C#N)n3nccc23)C[C@H](CN2CCC3(CCCNC3)C2)O1]

The title compound was prepared in analogy to the preparation of Example 3 by using tert-butyl 2,7-diazaspiro[4.5]decane-7-carboxylate (CAS: 236406-61-4, Wuxi AppTech) instead of tert-butyl 3,9-diazaspiro[5.5]undecane-3-carboxylate. Example 25 (1.3 mg) was obtained as a light yellow solid. MS: calc'd 395 (MH⁺), measured 395 (MH⁺). ¹H NMR (400 MHz, METHANOL-d4) δ 8.05 (d, J=2.4 Hz, 1H), 7.50 (d, J=7.9 Hz, 1H), 6.90 (dd, J=1.1, 2.3 Hz, 1H), 6.64 (d, J=8.1 Hz, 1H), 4.24 (br t, J=9.9 Hz, 1H), 4.08-3.97 (m, 1H), 3.79 (br t, J=10.8 Hz, 4H), 3.54-3.37 (m, 3H), 3.30-3.07 (m, 5H), 2.78-2.66 (m, 2H), 2.31-1.98 (m, 2H), 1.88 (br s, 4H), 1.33 (dd, J=1.2, 6.2 Hz, 3H).

### Example 26

### 4-[(2S,6R)-2-(2,7-diazaspiro[3.5]nonan-7-ylmethyl)-6-methyl-morpholin-4-yl]pyrazolo[1,5-a]pyridine-7-carbonitrile

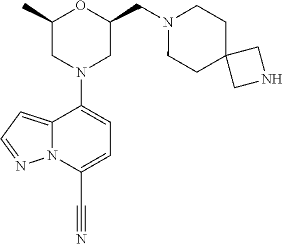 [SMILES: C[C@@H]1CN(c2ccc(C#N)n3nccc23)C[C@H](CN2CCC3(CC2)CNC3)O1]

The title compound was prepared in analogy to the preparation of Example 3 by using tert-butyl 2,7-diazaspiro[3.5]nonane-2-carboxylate (CAS: 236406-55-6, PharmaBlock) instead of tert-butyl 3,9-diazaspiro[5.5]undecane-3-carboxylate. Example 26 (1.3 mg) was obtained as a light yellow solid. MS: calc'd 381 (MH⁺), measured 381 (MH⁺). ¹H NMR (400 MHz, METHANOL-d4) δ 8.03 (d, J=2.3 Hz, 1H), 7.47 (d, J=7.9 Hz, 1H), 6.88 (d, J=2.4 Hz, 1H), 6.62 (d, J=8.1 Hz, 1H), 4.36-4.26 (m, 1H), 4.11-3.85 (m, 5H), 3.77 (td, J=2.5, 12.6 Hz, 2H), 3.74-3.54 (m, 2H), 3.39-3.32 (m, 1H), 3.30-3.24 (m, 1H), 3.23-3.02 (m, 2H), 2.78-2.64 (m, 2H), 2.41-1.98 (m, 4H), 1.31 (d, J=6.2 Hz, 3H).

### Example 27

### 4-[(2S,6R)-2-(2,9-diazaspiro[5.5]undecan-9-ylmethyl)-6-methyl-morpholin-4-yl]pyrazolo[1,5-a]pyridine-7-carbonitrile

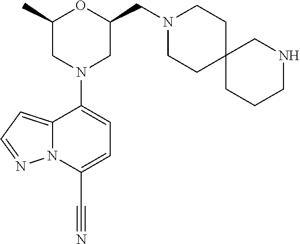 [SMILES: C[C@@H]1CN(c2ccc(C#N)n3nccc23)C[C@H](CN2CCC3(CCCNC3)CC2)O1]

The title compound was prepared in analogy to the preparation of Example 3 by using tert-butyl 2,9-diazaspiro[5.5]undecane-2-carboxylate (CAS: 189333-03-7, PharmaBlock) instead of tert-butyl 3,9-diazaspiro[5.5]undecane-3-carboxylate. Example 27 (13 mg) was obtained as a yellow solid. MS: calc'd 409 (MH⁺), measured 409 (MH⁺). ¹H NMR (400 MHz, METHANOL-d4) δ 8.04 (d, J=2.4 Hz, 1H), 7.48 (d, J=7.9 Hz, 1H), 6.89 (d, J=2.4 Hz, 1H), 6.63 (d, J=8.1 Hz, 1H), 4.32 (br t, J=9.8 Hz, 1H), 4.08-3.97 (m, 1H), 3.84-3.73 (m, 2H), 3.72-3.50 (m, 2H), 3.44-3.33 (m, 2H), 3.30-3.11 (m, 5H), 3.02 (br s, 1H), 2.79-2.66 (m, 2H), 2.14-1.57 (m, 8H), 1.31 (d, J=6.2 Hz, 3H).

### Example 28

### 4-[(2R,6S)-2-methyl-6-[(3-methylpiperazin-1-yl)methyl]morpholin-4-yl]pyrazolo[1,5-a]pyridine-7-carbonitrile

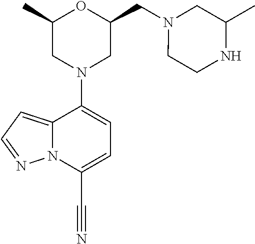 [SMILES: CC1CN(C[C@H]2CN(c3ccc(C#N)n4nccc34)C[C@@H](C)O2)CCN1]

The title compound was prepared in analogy to the preparation of Example 3 by using tert-butyl 2-methylpiperazine-1-carboxylate instead of tert-butyl 3,9-diazaspiro[5.5]undecane-3-carboxylate. Example 28 (19 mg) was obtained as a light yellow solid. MS: calc'd 355 (MH⁺), measured 355 (MH⁺). ¹H NMR (400 MHz, METHANOL-d4) δ 8.03 (d, J=2.3 Hz, 1H), 7.47 (d, J=8.1 Hz, 1H), 6.87 (dd, J=1.0, 2.3 Hz, 1H), 6.60 (d, J=8.1 Hz, 1H), 4.22-4.12 (m, 1H), 4.02-3.92 (m, 1H), 3.81 (br d, J=12.2 Hz, 1H), 3.75 (br d, J=12.3 Hz, 1H), 3.64-3.44 (m, 4H), 3.42-3.33 (m, 1H), 3.07-2.85 (m, 3H), 2.80-2.63 (m, 3H), 1.37 (d, J=6.6 Hz, 3H), 1.28 (d, J=6.2 Hz, 3H)

### Example 29

### 4-[(2S,6R)-2-[(4-amino-4-methyl-1-piperidyl)methyl]-6-methyl-morpholin-4-yl]pyrazolo[1,5-a]pyridine-7-carbonitrile

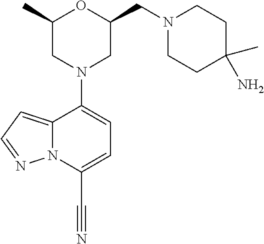 [SMILES: C[C@@H]1CN(c2ccc(C#N)n3nccc23)C[C@H](CN2CCC(C)(N)CC2)O1]

The title compound was prepared in analogy to the preparation of Example 3 by using tert-butyl 2-methylpiperazine-1-carboxylate instead of tert-butyl 3,9-diazaspiro[5.5]undecane-3-carboxylate. Example 29 (19 mg) was obtained as a light yellow solid. MS: calc'd 369 (MH⁺), measured 369 (MH⁺). ¹H NMR (400 MHz, METHANOL-d4) δ 8.04 (d, J=2.4 Hz, 1H), 7.48 (d, J=8.1 Hz, 1H), 6.90 (d, J=2.4 Hz, 1H), 6.63 (d, J=7.9 Hz, 1H), 4.38-4.29 (m, 1H), 4.08-3.97 (m, 1H), 3.79 (br t, J=13.9 Hz, 2H), 3.75-3.52 (m, 2H), 3.50-3.33 (m, 4H), 2.79-2.67 (m, 2H), 2.33-2.17 (m, 2H), 2.17-2.06 (m, 2H), 1.55 (s, 3H), 1.31 (d, J=6.2 Hz, 3H).

### Example 30

### 4-[(2S,6R)-2-(2,5-diazabicyclo[2.2.2]octan-2-ylmethyl)-6-methyl-morpholin-4-yl]pyrazolo[1,5-a]pyridine-7-carbonitrile

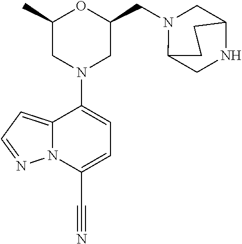 [SMILES: C[C@@H]1CN(c2ccc(C#N)n3nccc23)C[C@H](CN2CC3CCC2CN3)O1]

The title compound was prepared in analogy to the preparation of Example 3 by using tert-butyl 2,5-diazabicyclo[2.2.2]octane-2-carboxylate (CAS: 858671-91-7, PharmaBlock) instead of tert-butyl 3,9-diazaspiro[5.5]undecane-3-carboxylate. Example 30 (24 mg) was obtained as a light yellow solid. MS: calc'd 367 (MH⁺), measured 367 (MH⁺). ¹H NMR (400 MHz, METHANOL-d4) δ 8.08-8.02 (m, 1H), 7.49 (d, J=7.9 Hz, 1H), 6.90 (t, J=2.4 Hz, 1H), 6.63 (d, J=7.9 Hz, 1H), 4.27-4.16 (m, 1H), 4.04-3.93 (m, 1H), 3.89-3.71 (m, 5H), 3.70-3.53 (m, 2H), 3.48 (ddd, J=1.9, 6.5, 13.4 Hz, 1H), 3.43-3.33 (m, 2H), 2.81-2.65 (m, 2H), 2.49-2.33 (m, 1H), 2.25-2.12 (m, 1H), 2.11-1.91 (m, 2H), 1.30 (dd, J=1.1, 6.2 Hz, 3H).

### Example 31

### 4-[(2S,6R)-2-(3,8-diazabicyclo[3.2.1]octan-3-ylmethyl)-6-methyl-morpholin-4-yl]pyrazolo[1,5-a]pyridine-7-carbonitrile

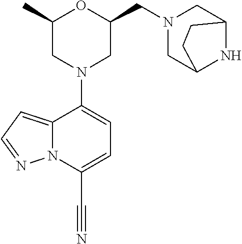 [SMILES: C[C@@H]1CN(c2ccc(C#N)n3nccc23)C[C@H](CN2CC3CCC(C2)N3)O1]

The title compound was prepared in analogy to the preparation of Example 3 by using tert-butyl 3,8-diazabicyclo[3.2.1]octane-8-carboxylate (CAS: 149771-44-8, PharmaBlock) instead of tert-butyl 3,9-diazaspiro[5.5]undecane-3-carboxylate. Example 30 (18 mg) was obtained as a light yellow solid. MS: calc'd 367 (MH⁺), measured 367 (MH⁺). ¹H NMR (400 MHz, METHANOL-d4) δ 8.03 (d, J=2.4 Hz, 1H), 7.47 (d, J=8.1 Hz, 1H), 6.85 (d, J=2.4 Hz, 1H), 6.60 (d, J=8.1 Hz, 1H), 4.05-3.97 (m, 3H), 3.92 (ddd, J=2.3, 6.3, 10.3 Hz, 1H), 3.88-3.81 (m, 1H), 3.75-3.68 (m, 1H), 3.16-3.08 (m, 1H), 3.04 (br d, J=11.2 Hz, 1H), 2.78-2.71 (m, 2H), 2.70-2.63 (m, 4H), 2.22-2.09 (m, 2H), 2.09-1.97 (m, 2H), 1.25 (d, J=6.2 Hz, 3H).

### Example 32

### 4-[(2S,6R)-2-(2,9-diazaspiro[5.5]undecan-2-ylmethyl)-6-methyl-morpholin-4-yl]pyrazolo[1,5-a]pyridine-7-carbonitrile

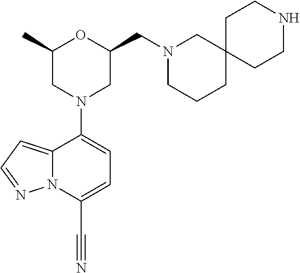 [SMILES: C[C@@H]1CN(c2ccc(C#N)n3nccc23)C[C@H](CN2CCCC3(CCNCC3)C2)O1]

The title compound was prepared in analogy to the preparation of Example 3 by using tert-butyl 2,9-diazaspiro[5.5]undecane-9-carboxylate (CAS: 1023595-19-8, PharmaBlock) instead of tert-butyl 3,9-diazaspiro[5.5]undecane-3-carboxylate. Example 32 (24 mg) was obtained as a light yellow solid. MS: calc'd 409 (MH⁺), measured 409 (MH⁺). ¹H NMR (400 MHz, METHANOL-d4) δ 8.04 (d, J=2.3 Hz, 1H), 7.48 (d, J=8.1 Hz, 1H), 6.88 (d, J=2.4 Hz, 1H), 6.63 (d, J=7.9 Hz, 1H), 4.40 (br t, J=10.0 Hz, 1H), 4.12-3.98 (m, 1H), 3.85-3.74 (m, 2H), 3.74-3.60 (m, 1H), 3.59-3.48 (m, 1H), 3.45-3.34 (m, 1H), 3.30-3.19 (m, 5H), 3.15-2.87 (m, 2H), 2.79-2.68 (m, 2H), 2.20-1.89 (m, 5H), 1.84-1.63 (m, 2H), 1.63-1.38 (m, 1H), 1.31 (d, J=6.2 Hz, 3H).

### Example 33

### 4-[(2S,6R)-2-(2,6-diazaspiro[3.5]nonan-6-ylmethyl)-6-methyl-morpholin-4-yl]pyrazolo[1,5-a]pyridine-7-carbonitrile

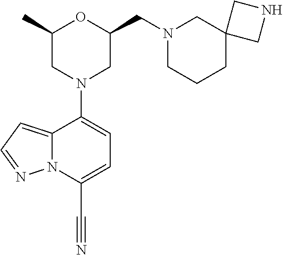 [SMILES: C[C@@H]1CN(c2ccc(C#N)n3nccc23)C[C@H](CN2CCCC3(CNC3)C2)O1]

The title compound was prepared in analogy to the preparation of Example 3 by using tert-butyl 2,8-diazaspiro[3.5]nonane-2-carboxylate (CAS: 1086394-57-1, PharmaBlock) instead of tert-butyl 3,9-diazaspiro[5.5]undecane-3-carboxylate. Example 33 (13 mg) was obtained as a light brown semisolid. MS: calc'd 381 (MH⁺), measured 381 (MH⁺). ¹H NMR (400 MHz, METHANOL-d4) δ 8.03 (d, J=2.3 Hz, 1H), 7.47 (d, J=8.1 Hz, 1H), 6.83 (d, J=2.4 Hz, 1H), 6.59 (d, J=8.1 Hz, 1H), 4.07-3.89 (m, 2H), 3.88-3.70 (m, 6H), 2.82-2.51 (m, 7H), 2.51-2.32 (m, 1H), 1.82-1.66 (m, 2H), 1.66-1.55 (m, 2H), 1.27 (d, J=6.2 Hz, 3H).

### Example 34

### 4-[(2S,6R)-2-[(4-amino-3,3-difluoro-1-piperidyl)methyl]-6-methyl-morpholin-4-yl]pyrazolo[1,5-a]pyridine-7-carbonitrile

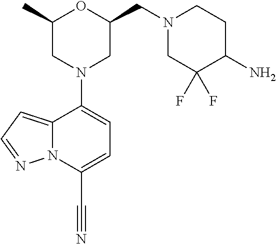 [SMILES: C[C@@H]1CN(c2ccc(C#N)n3nccc23)C[C@H](CN2CCC(N)C(F)(F)C2)O1]

The title compound was prepared in analogy to the preparation of Example 3 by using tert-butyl N-(3,3-difluoro-4-piperidyl)carbamate instead of tert-butyl 3,9-diazaspiro[5.5]undecane-3-carboxylate. Example 34 (9 mg) was obtained as a light yellow solid. MS: calc'd 391 (MH⁺), measured 391 (MH⁺). ¹H NMR (400 MHz, METHANOL-d4) δ 8.02 (t, J=2.2 Hz, 1H), 7.47 (d, J=7.9 Hz, 1H), 6.85 (d, J=1.7 Hz, 1H), 6.58 (dd, J=1.3, 8.1 Hz, 1H), 4.04-3.96 (m, 1H), 3.95-3.80 (m, 2H), 3.72 (br t, J=12.2 Hz, 1H), 3.50-3.33 (m, 2H), 3.15-2.95 (m, 1H), 2.77-2.53 (m, 5H), 2.53-2.33 (m, 1H), 2.07-1.95 (m, 1H), 1.84-1.71 (m, 1H), 1.25 (d, J=6.2 Hz, 3H).

### Example 35

### 4-[(2S,6R)-2-[(4-amino-3,3-difluoro-pyrrolidin-1-yl)methyl]-6-methyl-morpholin-4-yl]pyrazolo[1,5-a]pyridine-7-carbonitrile

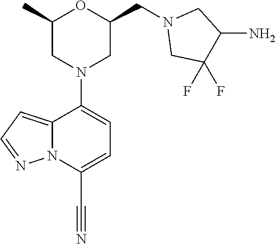 [SMILES: C[C@@H]1CN(c2ccc(C#N)n3nccc23)C[C@H](CN2CC(N)C(F)(F)C2)O1]

The title compound was prepared in analogy to the preparation of Example 3 by using tert-butyl N-(4,4-difluoropyrrolidin-3-yl)carbamate instead of tert-butyl 3,9-diazaspiro[5.5]undecane-3-carboxylate. Example 35 (14 mg) was obtained as a light yellow solid. MS: calc'd 377 (MH⁺), measured 377 (MH⁺). ¹H NMR (400 MHz, METHANOL-d4) δ 8.02 (d, J=2.3 Hz, 1H), 7.46 (d, J=7.9 Hz, 1H), 6.85 (t, J=2.1 Hz, 1H), 6.59 (d, J=7.9 Hz, 1H), 4.02-3.86 (m, 2H), 3.81-3.72 (m, 2H), 3.70-3.55 (m, 1H), 3.29-3.17 (m, 2H), 3.04 (qd, J=12.1, 16.5 Hz, 1H), 2.79-2.55 (m, 5H), 1.26 (d, J=6.2 Hz, 3H)

### Example 36

### 4-[(2S,6R)-2-[(8-amino-3-azabicyclo[3.2.1]octan-3-yl)methyl]-6-methyl-morpholin-4-yl]pyrazolo[1,5-a]pyridine-7-carbonitrile

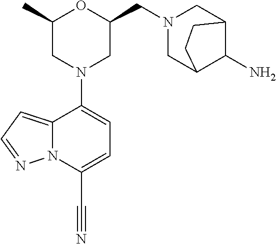 [SMILES: C[C@@H]1CN(c2ccc(C#N)n3nccc23)C[C@H](CN2CC3CCC(C2)C3N)O1]

The title compound was prepared in analogy to the preparation of Example 3 by using tert-butyl N-(3-azabicyclo[3.2.1]octan-8-yl)carbamate (CAS: 198210-17-2, PharmaBlock) instead of tert-butyl 3,9-diazaspiro[5.5]undecane-3-carboxylate. Example 36 (13 mg) was obtained as a light yellow solid. MS: calc'd 381 (MH⁺), measured 381 (MH⁺). ¹H NMR (400 MHz, METHANOL-d4) δ 8.03 (d, J=2.3 Hz, 1H), 7.48 (d, J=7.9 Hz, 1H), 6.86 (d, J=2.4 Hz, 1H), 6.62 (d, J=7.9 Hz, 1H), 4.31 (br t, J=9.6 Hz, 1H), 4.05-3.95 (m, 1H), 3.77 (br dd, J=12.2, 19.6 Hz, 2H), 3.63 (br d, J=14.1 Hz, 1H), 3.53-3.32 (m, 6H), 2.80-2.66 (m, 2H), 2.58 (br s, 2H), 2.18-2.00 (m, 3H), 1.96-1.86 (m, 1H), 1.29 (d, J=6.2 Hz, 3H).

### Example 37

### 4-[(2S,6R)-2-[(3-amino-8-azabicyclo[3.2.1]octan-8-yl)methyl]-6-methyl-morpholin-4-yl]pyrazolo[1,5-a]pyridine-7-carbonitrile

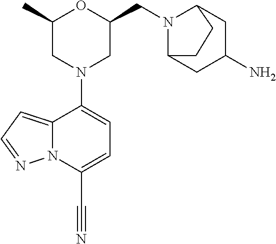 [SMILES: C[C@@H]1CN(c2ccc(C#N)n3nccc23)C[C@H](CN2C3CCC2CC(N)C3)O1]

The title compound was prepared in analogy to the preparation of Example 3 by using tert-butyl N-(8-azabicyclo[3.2.1]octan-3-yl)carbamate (CAS: 287114-25-4, PharmaBlock) instead of tert-butyl 3,9-diazaspiro[5.5]undecane-3-carboxylate. Example 37 (13 mg) was obtained as a light yellow solid. MS: calc'd 381 (MH⁺), measured 381 (MH⁺). ¹H NMR (400 MHz, METHANOL-d4) δ 8.03 (d, J=2.3 Hz, 1H), 7.48 (d, J=7.9 Hz, 1H), 6.88 (d, J=2.3 Hz, 1H), 6.60 (d, J=7.9 Hz, 1H), 4.10 (br s, 1H), 4.00-3.92 (m, 1H), 3.89 (br d, J=12.1 Hz, 1H), 3.84 (br s, 1H), 3.74 (br d, J=12.3 Hz, 1H), 3.69 (br s, 1H), 3.56-3.45 (m, 1H), 2.97-2.87 (m, 1H), 2.87-2.78 (m, 1H), 2.76-2.62 (m, 2H), 2.25-2.10 (m, 2H), 2.02-1.90 (m, 4H), 1.86-1.76 (m, 2H), 1.27 (d, J=6.2 Hz, 3H).

### Example 38

### 4-[(2R,6S)-2-methyl-6-[[(3R)-3-phenylpiperazin-1-yl]methyl]morpholin-4-yl]pyrazolo[1,5-a]pyridine-7-carbonitrile

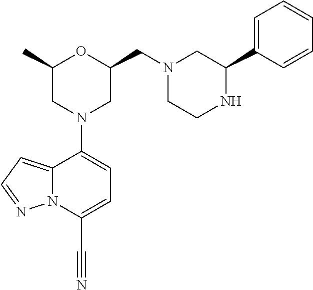 [SMILES: C[C@@H]1CN(c2ccc(C#N)n3nccc23)C[C@H](CN2CCN[C@H](c3ccccc3)C2)O1]

The title compound was prepared in analogy to the preparation of Example 3 by using tert-butyl (2R)-2-phenylpiperazine-1-carboxylate (CAS: 859518-32-4, PharmaBlock) instead of tert-butyl 3,9-diazaspiro[5.5]undecane-3-carboxylate. Example 38 (18 mg) was obtained as a light yellow solid. MS: calc'd 417 (MH⁺), measured 417 (MH⁺). ¹H NMR (400 MHz, METHANOL-d4) δ 8.03 (d, J=2.4 Hz, 1H), 7.51-7.38 (m, 6H), 6.86 (d, J=2.4 Hz, 1H), 6.60 (d, J=8.1 Hz, 1H), 4.29 (dd, J=3.0, 10.9 Hz, 1H), 4.09-4.00 (m, 1H), 3.97-3.87 (m, 1H), 3.82 (br d, J=12.3 Hz, 1H), 3.75 (br d, J=12.2 Hz, 1H), 3.38-3.31 (m, 2H), 3.28-3.25 (m, 1H), 3.19 (br d, J=12.7 Hz, 1H), 2.80-2.73 (m, 1H), 2.73-2.68 (m, 2H), 2.68-2.63 (m, 1H), 2.63-2.54 (m, 2H), 1.23 (d, J=6.2 Hz, 3H).

### Example 39

### 4-[(2R,6S)-2-methyl-6-[(4-piperazin-1-yl-1-piperidyl)methyl]morpholin-4-yl]pyrazolo[1,5-a]pyridine-7-carbonitrile

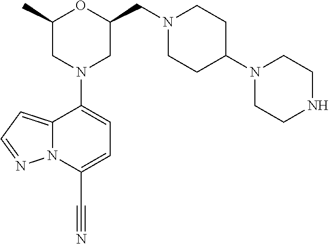 [SMILES: C[C@@H]1CN(c2ccc(C#N)n3nccc23)C[C@H](CN2CCC(N3CCNCC3)CC2)O1]

The title compound was prepared in analogy to the preparation of Example 3 by using tert-butyl 4-(4-piperidyl)piperazine-1-carboxylate instead of tert-butyl 3,9-diazaspiro[5.5]undecane-3-carboxylate. Example 39 (13 mg) was obtained as a light yellow solid. MS: calc'd 424 (MH⁺), measured 424 (MH⁺). ¹H NMR (400 MHz, METHANOL-d4) δ 8.03 (d, J=2.3 Hz, 1H), 7.47 (d, J=7.9 Hz, 1H), 6.89 (d, J=2.4 Hz, 1H), 6.62 (d, J=8.1 Hz, 1H), 4.26 (br t, J=9.6 Hz, 1H), 4.05-3.95 (m, 1H), 3.78 (br t, J=12.2 Hz, 2H), 3.69-3.52 (m, 2H), 3.28-3.13 (m, 6H), 3.11-2.97 (m, 2H), 2.88-2.78 (m, 4H), 2.77-2.62 (m, 3H), 2.14-2.00 (m, 2H), 1.98-1.79 (m, 2H), 1.30 (d, J=6.2 Hz, 3H)

### Example 40

### 4-[(2S,6R)-2-(4,7-diazaspiro[2.5]octan-7-ylmethyl)-6-methyl-morpholin-4-yl]pyrazolo[1,5-a]pyridine-7-carbonitrile

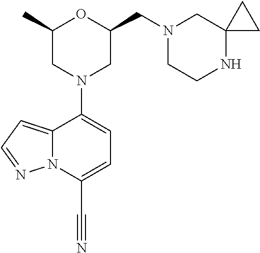 [SMILES: C[C@@H]1CN(c2ccc(C#N)n3nccc23)C[C@H](CN2CCNC3(CC3)C2)O1]

The title compound was prepared in analogy to the preparation of Example 3 by using tert-butyl 4,7-diazaspiro[2.5]octane-4-carboxylate instead of tert-butyl 3,9-diazaspiro[5.5]undecane-3-carboxylate. Example 40 (12 mg) was obtained as a light yellow solid. MS: calc'd 367 (MH⁺), measured 367 (MH⁺). ¹H NMR (400 MHz, METHANOL-d4) δ 8.03 (d, J=2.3 Hz, 1H), 7.47 (d, J=7.9 Hz, 1H), 6.86 (d, J=2.4 Hz, 1H), 6.59 (d, J=8.1 Hz, 1H), 4.11-4.02 (m, 1H), 3.98-3.88 (m, 1H), 3.82 (br d, J=12.3 Hz, 1H), 3.74 (br d, J=12.2 Hz, 1H), 3.29-3.24 (m, 2H), 3.07-2.96 (m, 1H), 2.95-2.88 (m, 1H), 2.88-2.80 (m, 1H), 2.79-2.69 (m, 4H), 2.66 (dd, J=10.5, 12.3 Hz, 1H), 1.26 (d, J=6.2 Hz, 3H), 0.99-0.91 (m, 2H), 0.90-0.82 (m, 2H).

### Example 41

### 4-[(2S,6R)-2-(1,2,3,3a,4,6,7,7a-octahydropyrrolo[3,2-c]pyridin-5-ylmethyl)-6-methyl-morpholin-4-yl]pyrazolo[1,5-a]pyridine-7-carbonitrile

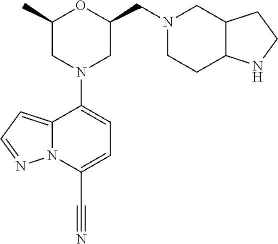 [SMILES: C[C@@H]1CN(c2ccc(C#N)n3nccc23)C[C@H](CN2CCC3NCCC3C2)O1]

The title compound was prepared in analogy to the preparation of Example 3 by using tert-butyl 2,3,3a,4,5,6,7,7a-octahydropyrrolo[3,2-c]pyridine-1-carboxylate (CAS: 1147422-00-1, PharmaBlock) instead of tert-butyl 3,9-diazaspiro[5.5]undecane-3-carboxylate. Example 41 (14 mg) was obtained as a light yellow solid. MS: calc'd 381 (MH⁺), measured 381 (MH⁺). ¹H NMR (400 MHz, METHANOL-d4) δ 8.03 (d, J=2.3 Hz, 1H), 7.47 (d, J=8.1 Hz, 1H), 6.91-6.83 (m, 1H), 6.61 (d, J=8.1 Hz, 1H), 4.31-4.16 (m, 1H), 4.04-3.93 (m, 1H), 3.91-3.79 (m, 2H), 3.75 (td, J=2.0, 12.4 Hz, 1H), 3.58-3.47 (m, 1H), 3.46-3.33 (m, 2H), 3.29-2.99 (m, 5H), 2.82-2.65 (m, 3H), 2.33-2.20 (m, 2H), 2.19-2.07 (m, 2H), 1.33-1.26 (m, 3H).

### Example 42

### 4-[(2S,6R)-2-(2,6-diazaspiro[4.5]decan-2-ylmethyl)-6-methyl-morpholin-4-yl]pyrazolo[1,5-a]pyridine-7-carbonitrile

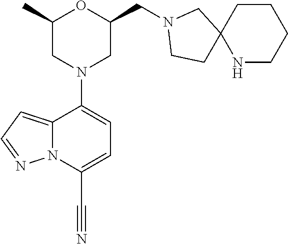 [SMILES: C[C@@H]1CN(c2ccc(C#N)n3nccc23)C[C@H](CN2CCC3(CCCCN3)C2)O1]

The title compound was prepared in analogy to the preparation of Example 3 by using tert-butyl 2,6-diazaspiro[4.5]decane-6-carboxylate (CAS: 960294-16-0, PharmaBlock) instead of tert-butyl 3,9-diazaspiro[5.5]undecane-3-carboxylate. Example 42 (10 mg) was obtained as a light yellow solid. MS: calc'd 395 (MH⁺), measured 395 (MH⁺). ¹H NMR (400 MHz, METHANOL-d4) δ 8.02 (d, J=2.2 Hz, 1H), 7.46 (d, J=7.9 Hz, 1H), 6.85 (d, J=2.3 Hz, 1H), 6.59 (d, J=7.9 Hz, 1H), 4.05-3.90 (m, 2H), 3.77 (br d, J=12.7 Hz, 2H), 3.30-3.13 (m, 4H), 2.84-2.72 (m, 3H), 2.71-2.56 (m, 3H), 2.17-1.99 (m, 2H), 1.89-1.82 (m, 2H), 1.82-1.66 (m, 4H), 1.27 (dd, J=3.8, 6.2 Hz, 3H).

### Example 43

### 4-[(2S,6R)-2-(3,8-diazabicyclo[3.2.1]octan-8-ylmethyl)-6-methyl-morpholin-4-yl]pyrazolo[1,5-a]pyridine-7-carbonitrile

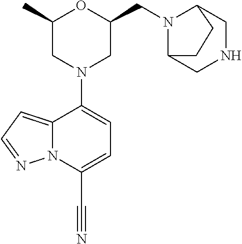 [SMILES: C[C@@H]1CN(c2ccc(C#N)n3nccc23)C[C@H](CN2C3CCC2CNC3)O1]

The title compound was prepared in analogy to the preparation of Example 3 by using tert-butyl 3,8-diazabicyclo[3.2.1]octane-3-carboxylate instead of tert-butyl 3,9-diazaspiro[5.5]undecane-3-carboxylate. Example 43 (5 mg) was obtained as a light yellow solid. MS: calc'd 367 (MH⁺), measured 367 (MH⁺). ¹H NMR (400 MHz, METHANOL-d4) δ 8.03 (d, J=2.4 Hz, 1H), 7.48 (d, J=7.9 Hz, 1H), 6.87 (d, J=2.3 Hz, 1H), 6.60 (d, J=8.1 Hz, 1H), 4.00-3.85 (m, 3H), 3.78-3.69 (m, 1H), 3.66-3.58 (m, 1H), 3.55-3.49 (m, 1H), 3.29-3.21 (m, 2H), 3.14-3.04 (m, 2H), 2.76 (dd, J=10.5, 12.2 Hz, 1H), 2.71-2.62 (m, 2H), 2.60-2.53 (m, 1H), 2.30-2.14 (m, 2H), 1.93-1.84 (m, 2H), 1.26 (d, J=6.2 Hz, 3H).

### Example 44

### 4-[(2S,6R)-2-(2,7-diazaspiro[4.5]decan-7-ylmethyl)-6-methyl-morpholin-4-yl]pyrazolo[1,5-a]pyridine-7-carbonitrile

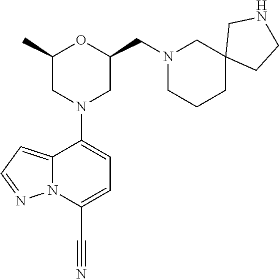 [SMILES: C[C@@H]1CN(c2ccc(C#N)n3nccc23)C[C@H](CN2CCCC3(CCNC3)C2)O1]

The title compound was prepared in analogy to the preparation of Example 3 by using tert-butyl 2,9-diazaspiro[4.5]decane-2-carboxylate (CAS: 885268-42-8, Wuxi AppTech) instead of tert-butyl 3,9-diazaspiro[5.5]undecane-3-carboxylate. Example 44 (12 mg) was obtained as a light brown solid. MS: calc'd 395 (MH⁺), measured 395 (MH⁺). ¹H NMR (400 MHz, METHANOL-d4) δ 8.04 (d, J=2.3 Hz, 1H), 7.48 (d, J=7.9 Hz, 1H), 6.83 (dd, J=2.3, 5.7 Hz, 1H), 6.60 (dd, J=2.6, 8.1 Hz, 1H), 4.01-3.82 (m, 3H), 3.74 (br t, J=11.9 Hz, 1H), 3.36-3.33 (m, 3H), 3.28-3.18 (m, 1H), 3.11-3.03 (m, 1H), 2.75-2.59 (m, 3H), 2.58-2.44 (m, 3H), 2.38-2.28 (m, 1H), 2.05-1.91 (m, 1H), 1.87-1.77 (m, 1H), 1.75-1.55 (m, 3H), 1.54-1.43 (m, 1H), 1.25 (d, J=6.2 Hz, 3H).

### Example 45

### 4-[(2S,6R)-2-(1,2,3,4,4a,5,7,7a-octahydropyrrolo[3,4-b]pyridin-6-ylmethyl)-6-methyl-morpholin-4-yl]pyrazolo[1,5-a]pyridine-7-carbonitrile

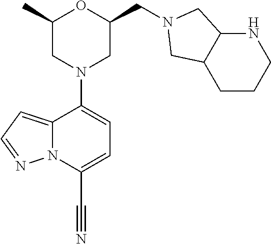 [SMILES: C[C@@H]1CN(c2ccc(C#N)n3nccc23)C[C@H](CN2CC3CCCNC3C2)O1]

The title compound was prepared in analogy to the preparation of Example 3 by using tert-butyl 2,3,4,4a,5,6,7,7a-octahydropyrrolo[3,4-b]pyridine-1-carboxylate (CAS: 159877-36-8, Accela) instead of tert-butyl 3,9-diazaspiro[5.5]undecane-3-carboxylate. Example 45 (12 mg) was obtained as a light brown solid. MS: calc'd 381 (MH⁺), measured 381 (MH⁺). ¹H NMR (400 MHz, METHANOL-d4) δ 8.06 (d, J=2.3 Hz, 1H), 7.50 (d, J=8.1 Hz, 1H), 6.91-6.87 (m, 1H), 6.63 (d, J=8.1 Hz, 1H), 4.08-3.91 (m, 2H), 3.86-3.75 (m, 2H), 3.64 (q, J=4.5 Hz, 1H), 3.30-3.23 (m, 1H), 3.11-2.61 (m, 10H), 1.88-1.73 (m, 4H), 1.30 (t, J=6.7 Hz, 3H).

### Example 46

### 4-[(2S,6R)-2-(2,8-diazaspiro[3.6]decan-2-ylmethyl)-6-methyl-morpholin-4-yl]pyrazolo[1,5-a]pyridine-7-carbonitrile

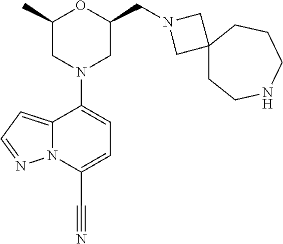 [SMILES: C[C@@H]1CN(c2ccc(C#N)n3nccc23)C[C@H](CN2CC3(CCCNCC3)C2)O1]

The title compound was prepared in analogy to the preparation of Example 3 by using tert-butyl 2,8-diazaspiro[3.6]decane-8-carboxylate (CAS: 270257-46-0, Wuxi AppTech) instead of tert-butyl 3,9-diazaspiro[5.5]undecane-3-carboxylate. Example 46 (13 mg) was obtained as a light brown solid. MS: calc'd 395 (MH⁺), measured 395 (MH⁺). ¹H NMR (400 MHz, METHANOL-d4) δ 8.03 (d, J=2.4 Hz, 1H), 7.48 (d, J=7.9 Hz, 1H), 6.87 (d, J=2.4 Hz, 1H), 6.60 (d, J=7.9 Hz, 1H), 4.01-3.89 (m, 2H), 3.81 (br d, J=12.5 Hz, 1H), 3.76 (br d, J=12.3 Hz, 1H), 3.58-3.46 (m, 2H), 3.30-3.21 (m, 3H), 3.19-3.14 (m, 1H), 2.85-2.77 (m, 4H), 2.77-2.70 (m, 1H), 2.66 (dd, J=10.5, 12.2 Hz, 1H), 2.15-2.01 (m, 2H), 2.00-1.89 (m, 2H), 1.80-1.66 (m, 2H), 1.27 (d, J=6.2 Hz, 3H).

### Example 47

### 4-[(2S,6R)-2-(1,9-diazaspiro[5.5]undecan-9-ylmethyl)-6-methyl-morpholin-4-yl]pyrazolo[1,5-a]pyridine-7-carbonitrile

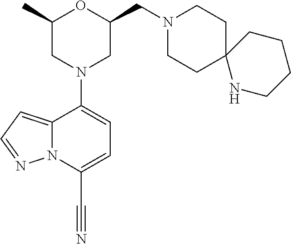 [SMILES: C[C@@H]1CN(c2ccc(C#N)n3nccc23)C[C@H](CN2CCC3(CCCCN3)CC2)O1]

The title compound was prepared in analogy to the preparation of Example 3 by using tert-butyl 1,9-diazaspiro[5.5]undecane-1-carboxylate (CAS: 1158750-00-5, PharmaBlock) instead of tert-butyl 3,9-diazaspiro[5.5]undecane-3-carboxylate. Example 47 (13 mg) was obtained as a light brown solid. MS: calc'd 409 (MH⁺), measured 409 (MH⁺). ¹H NMR (400 MHz, METHANOL-d4) δ 8.03 (d, J=2.4 Hz, 1H), 7.48 (d, J=8.1 Hz, 1H), 6.85 (d, J=2.6 Hz, 1H), 6.59 (d, J=8.1 Hz, 1H), 4.06-3.97 (m, 1H), 3.96-3.87 (m, 1H), 3.82 (br d, J=12.3 Hz, 1H), 3.74 (br d, J=12.2 Hz, 1H), 3.17 (t, J=5.7 Hz, 2H), 2.99-2.91 (m, 1H), 2.90-2.80 (m, 1H), 2.73-2.58 (m, 4H), 2.57-2.45 (m, 2H), 2.06-1.97 (m, 2H), 1.91-1.81 (m, 4H), 1.80-1.69 (m, 4H), 1.26 (d, J=6.2 Hz, 3H)

### Example 48

### 4-[(2S,6R)-2-(1,7-diazaspiro[3.5]nonan-7-ylmethyl)-6-methyl-morpholin-4-yl]pyrazolo[1,5-a]pyridine-7-carbonitrile

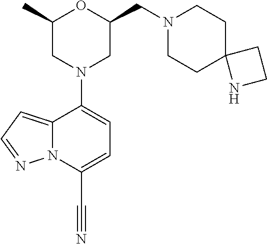 [SMILES: C[C@@H]1CN(c2ccc(C#N)n3nccc23)C[C@H](CN2CCC3(CCN3)CC2)O1]

The title compound was prepared in analogy to the preparation of Example 3 by using tert-butyl 1,7-diazaspiro[3.5]nonane-1-carboxylate (CAS: 1216936-29-6, PharmaBlock) instead of tert-butyl 3,9-diazaspiro[5.5]undecane-3-carboxylate. Example 47 (13 mg) was obtained as a light brown solid. MS: calc'd 381 (MH⁺), measured 381 (MH⁺). ¹H NMR (400 MHz, METHANOL-d4) δ 8.05 (d, J=2.3 Hz, 1H), 7.49 (d, J=7.9 Hz, 1H), 6.87 (d, J=2.4 Hz, 1H), 6.61 (d, J=7.9 Hz, 1H), 4.04-3.90 (m, 4H), 3.82 (br d, J=12.2 Hz, 1H), 3.77 (br d, J=12.3 Hz, 1H), 2.81-2.48 (m, 8H), 2.48-2.40 (m, 2H), 2.20-2.07 (m, 4H), 1.27 (d, J=6.2 Hz, 3H)

### Example 49

### 4-[(2S,6R)-2-(1,3,3a,4,5,6,7,7a-octahydropyrrolo[3,4-c]pyridin-2-ylmethyl)-6-methyl-morpholin-4-yl]pyrazolo[1,5-a]pyridine-7-carbonitrile

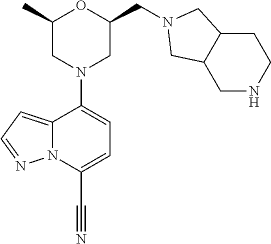 [SMILES: C[C@@H]1CN(c2ccc(C#N)n3nccc23)C[C@H](CN2CC3CCNCC3C2)O1]

The title compound was prepared in analogy to the preparation of Example 3 by using tert-butyl 1,2,3,3a,4,6,7,7a-octahydropyrrolo[3,4-c]pyridine-5-carboxylate (CAS: 351370-99-5, PharmaBlock) instead of tert-butyl 3,9-diazaspiro[5.5]undecane-3-carboxylate. Example 49 (17 mg) was obtained as a light yellow solid. MS: calc'd 381 (MH⁺), measured 381 (MH⁺). ¹H NMR (400 MHz, METHANOL-d4) δ 8.02 (d, J=2.2 Hz, 1H), 7.46 (d, J=7.9 Hz, 1H), 6.87 (d, J=2.0 Hz, 1H), 6.59 (d, J=7.9 Hz, 1H), 4.18-4.07 (m, 1H), 4.04-3.91 (m, 1H), 3.86-3.71 (m, 2H), 3.49-3.33 (m, 3H), 3.30-3.09 (m, 7H), 2.77-2.60 (m, 4H), 2.10-1.98 (m, 1H), 1.96-1.83 (m, 1H), 1.29 (d, J=6.2 Hz, 3H).

### Example 50

### 4-[(2R,6S)-2-methyl-6-[(2-methylpiperazin-1-yl)methyl]morpholin-4-yl]pyrazolo[1,5-a]pyridine-7-carbonitrile

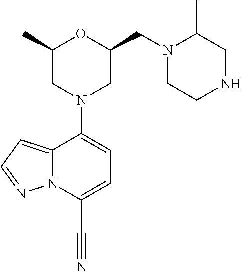 [SMILES: CC1CNCCN1C[C@H]1CN(c2ccc(C#N)n3nccc23)C[C@@H](C)O1]

The title compound was prepared in analogy to the preparation of Example 3 by using tert-butyl (3S)-3-methylpiperazine-1-carboxylate instead of tert-butyl 3,9-diazaspiro[5.5]undecane-3-carboxylate. Example 50A (8 mg) and Example 50B (7 mg) were obtained through prep-HPLC as light yellow powders.

Example 50 A: MS: calc'd 355 (MH⁺), measured 355 (MH⁺). ¹H NMR (400 MHz, METHANOL-d4) δ 8.02 (d, J=2.3 Hz, 1H), 7.45 (d, J=7.9 Hz, 1H), 6.84 (d, J=2.4 Hz, 1H), 6.58 (d, J=8.1 Hz, 1H), 4.03-3.94 (m, 1H), 3.94-3.85 (m, 1H), 3.76 (br d, J=12.0 Hz, 2H), 3.29-3.17 (m, 3H), 3.16-3.06 (m, 1H), 2.97 (dd, J=7.0, 14.2 Hz, 1H), 2.88-2.70 (m, 4H), 2.63 (dd, J=10.6, 12.3 Hz, 1H), 2.55 (dd, J=4.2, 14.2 Hz, 1H), 1.26 (d, J=6.2 Hz, 3H), 1.18 (d, J=5.7 Hz, 3H).

Example 50 B: MS: calc'd 355 (MH⁺), measured 355 (MH⁺). ¹H NMR (400 MHz, METHANOL-d4) δ 8.01 (d, J=2.3 Hz, 1H), 7.44 (d, J=7.9 Hz, 1H), 6.82 (d, J=2.4 Hz, 1H), 6.57 (d, J=7.9 Hz, 1H), 4.05-3.96 (m, 1H), 3.96-3.86 (m, 1H), 3.81 (br d, J=12.2 Hz, 1H), 3.75 (br d, J=12.3 Hz, 1H), 3.30-3.20 (m, 3H), 3.11 (dt, J=2.9, 11.8 Hz, 1H), 2.90 (dd, J=5.6, 13.8 Hz, 1H), 2.86-2.73 (m, 2H), 2.70-2.60 (m, 3H), 2.59-2.54 (m, 1H), 1.24 (d, J=6.2 Hz, 3H), 1.18 (d, J=5.9 Hz, 3H).

### Example 51

### 4-[(2S,6R)-2-[(4-amino-2-methyl-1-piperidyl)methyl]-6-methyl-morpholin-4-yl]pyrazolo[1,5-a]pyridine-7-carbonitrile

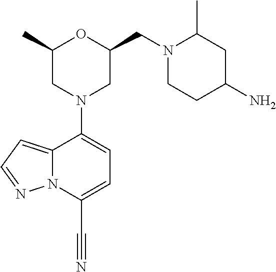 [SMILES: CC1CC(N)CCN1C[C@H]1CN(c2ccc(C#N)n3nccc23)C[C@@H](C)O1]

The title compound was prepared in analogy to the preparation of Example 3 by using tert-butyl 3-methylpiperazine-1-carboxylate (CAS: 1281674-64-3, PharmaBlock) instead of tert-butyl 3,9-diazaspiro[5.5]undecane-3-carboxylate. Example 51 (21 mg) was obtained as a light yellow powder. MS: calc'd 369 (MH⁺), measured 369 (MH⁺). ¹H NMR (400 MHz, METHANOL-d4) δ 8.04 (d, J=2.2 Hz, 1H), 7.48 (br d, J=7.8 Hz, 1H), 6.95-6.86 (m, 1H), 6.62 (br d, J=7.9 Hz, 1H), 4.31 (br t, J=9.2 Hz, 1H), 4.10-3.65 (m, 4H), 3.64-3.36 (m, 3H), 3.32-3.14 (m, 2H), 2.84-2.66 (m, 2H), 2.34-1.77 (m, 4H), 1.53-1.45 (m, 3H), 1.36-1.28 (m, 3H).

### Example 52

### 4-[(2S,6R)-2-(3,7-diazabicyclo[4.2.0]octan-7-ylmethyl)-6-methyl-morpholin-4-yl]pyrazolo[1,5-a]pyridine-7-carbonitrile

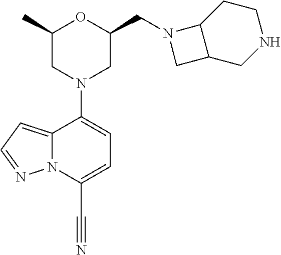 [SMILES: C[C@@H]1CN(c2ccc(C#N)n3nccc23)C[C@H](CN2CC3CNCCC32)O1]

The title compound was prepared in analogy to the preparation of Example 3 by using tert-butyl 3,7-diazabicyclo[4.2.0]octane-3-carboxylate (CAS: 885271-67-0, PharmaBlock) instead of tert-butyl 3,9-diazaspiro[5.5]undecane-3-carboxylate. Example 52 (24 mg) was obtained as a light yellow powder. MS: calc'd 367 (MH⁺), measured 367 (MH⁺). ¹H NMR (400 MHz, METHANOL-d4) δ 8.04 (d, J=2.2 Hz, 1H), 7.47 (d, J=7.9 Hz, 1H), 6.88 (d, J=1.8 Hz, 1H), 6.61 (d, J=7.9 Hz, 1H), 4.57 (br s, 1H), 4.21-3.90 (m, 4H), 3.83-3.72 (m, 2H), 3.72-3.63 (m, 1H), 3.56-3.35 (m, 4H), 3.31-3.19 (m, 1H), 3.12 (br s, 1H), 2.81-2.65 (m, 2H), 2.52-2.30 (m, 1H), 2.30-2.17 (m, 1H), 1.29 (dd, J=2.1, 6.2 Hz, 3H).

### Example 53

### 4-[(2S,6R)-2-(1,8-diazaspiro[4.5]decan-8-ylmethyl)-6-methyl-morpholin-4-yl]pyrazolo[1,5-a]pyridine-7-carbonitrile

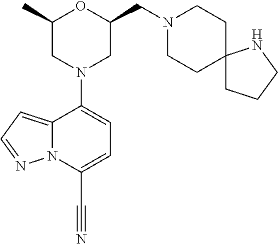 [SMILES: C[C@@H]1CN(c2ccc(C#N)n3nccc23)C[C@H](CN2CCC3(CCCN3)CC2)O1]

The title compound was prepared in analogy to the preparation of Example 3 by using tert-butyl 1,8-diazaspiro[4.5]decane-1-carboxylate (CAS: 885279-92-5, PharmaBlock) instead of tert-butyl 3,9-diazaspiro[5.5]undecane-3-carboxylate. Example 53 (24 mg) was obtained as a light yellow powder. MS: calc'd 395 (MH⁺), measured 395 (MH⁺). ¹H NMR (400 MHz, METHANOL-d4) δ 8.04 (d, J=2.4 Hz, 1H), 7.48 (d, J=7.9 Hz, 1H), 6.90 (d, J=2.4 Hz, 1H), 6.63 (d, J=8.1 Hz, 1H), 4.31 (br t, J=9.7 Hz, 1H), 4.07-3.98 (m, 1H), 3.85-3.70 (m, 2H), 3.70-3.51 (m, 2H), 3.44 (t, J=7.0 Hz, 2H), 3.32-3.22 (m, 4H), 2.73 (dt, J=10.5, 12.9 Hz, 2H), 2.38-2.25 (m, 2H), 2.25-2.11 (m, 6H), 1.32 (d, J=6.2 Hz, 3H).

### Example 54

### 4-[(2R,6S)-2-methyl-6-[(4-pyrrolidin-1-yl-1-piperidyl)methyl]morpholin-4-yl]pyrazolo[1,5-a]pyridine-7-carbonitrile

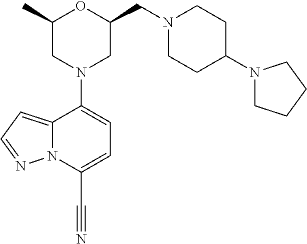 [SMILES: C[C@@H]1CN(c2ccc(C#N)n3nccc23)C[C@H](CN2CCC(N3CCCC3)CC2)O1]

The title compound was prepared in analogy to the preparation of Example 1 by using 4-pyrrolidin-1-ylpiperidine instead of 1-methyl-4-(piperidin-4-yl)piperazine hydrochloride. Example 54 (14 mg) was obtained as a light yellow viscous oil. MS: calc'd 409 (MH⁺), measured 409 (MH⁺). ¹H NMR (400 MHz, METHANOL-d4) δ 8.05 (d, J=2.3 Hz, 1H), 7.50 (d, J=8.1 Hz, 1H), 6.87 (d, J=2.4 Hz, 1H), 6.62 (d, J=7.9 Hz, 1H), 4.07-4.00 (m, 1H), 3.99-3.89 (m, 1H), 3.83 (br d, J=12.2 Hz, 1H), 3.76 (br d, J=12.2 Hz, 1H), 3.45-3.34 (m, 4H), 3.30-3.24 (m, 1H), 3.21-3.07 (m, 2H), 2.74-2.57 (m, 4H), 2.37-2.23 (m, 2H), 2.22-2.14 (m, 2H), 2.13-2.05 (m, 4H), 1.83-1.69 (m, 2H), 1.28 (d, J=6.2 Hz, 3H).

### Example 55

### 4-[(2S,6R)-2-(1,6-diazaspiro[3.3]heptan-1-ylmethyl)-6-methyl-morpholin-4-yl]pyrazolo[1,5-a]pyridine-7-carbonitrile

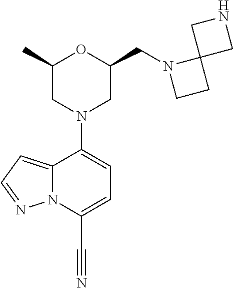 [SMILES: C[C@@H]1CN(c2ccc(C#N)n3nccc23)C[C@H](CN2CCC23CNC3)O1]

The title compound was prepared in analogy to the preparation of Example 3 by using tert-butyl 1,6-diazaspiro[3.3]heptane-6-carboxylate hemioxalate (CAS: 1272412-72-2, PharmaBlock) instead of tert-butyl 3,9-diazaspiro[5.5]undecane-3-carboxylate. Example 55 (10 mg) was obtained as a light yellow semisolid. MS: calc'd 353 (MH⁺), measured 353 (MH⁺). ¹H NMR (400 MHz, METHANOL-d4) δ 8.05 (d, J=2.4 Hz, 1H), 7.50 (d, J=8.1 Hz, 1H), 6.87 (d, J=2.4 Hz, 1H), 6.62 (d, J=8.1 Hz, 1H), 4.50-4.43 (m, 2H), 4.05-3.85 (m, 4H), 3.82-3.74 (m, 2H), 3.32-3.29 (m, 2H), 2.84 (d, J=5.6 Hz, 2H), 2.82-2.76 (m, 1H), 2.68 (dd, J=10.4, 12.5 Hz, 1H), 2.49 (t, J=6.9 Hz, 2H), 1.31 (d, J=6.2 Hz, 3H).

### Example 56

### 4-[(2S,6R)-2-(3,8-diazabicyclo[4.2.0]octan-8-ylmethyl)-6-methyl-morpholin-4-yl]pyrazolo[1,5-a]pyridine-7-carbonitrile

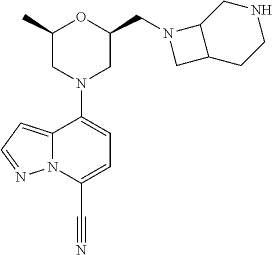 [SMILES: C[C@@H]1CN(c2ccc(C#N)n3nccc23)C[C@H](CN2CC3CCNCC32)O1]

The title compound was prepared in analogy to the preparation of Example 3 by using tert-butyl 3,8-diazabicyclo[4.2.0]octane-3-carboxylate (CAS: 928754-14-7, PharmaBlock) instead of tert-butyl 3,9-diazaspiro[5.5]undecane-3-carboxylate. Example 56 (18 mg) was obtained as a light yellow semisolid. MS: calc'd 367 (MH⁺), measured 367 (MH⁺). ¹H NMR (400 MHz, METHANOL-d4) δ 8.04 (t, J=2.0 Hz, 1H), 7.48 (d, J=7.9 Hz, 1H), 6.87 (dd, J=0.9, 2.4 Hz, 1H), 6.62 (dd, J=1.2, 7.9 Hz, 1H), 4.87-4.80 (m, 2H), 4.35-4.16 (m, 2H), 4.03-3.94 (m, 2H), 3.81-3.71 (m, 2H), 3.61-3.50 (m, 3H), 3.50-3.35 (m, 1H), 3.09-2.92 (m, 2H), 2.80-2.66 (m, 2H), 2.42 (ddd, J=3.1, 8.7, 15.1 Hz, 1H), 2.30-2.13 (m, 1H), 1.30 (dd, J=6.4, 7.6 Hz, 3H).

### Example 57

### 4-[(2R,6S)-2-methyl-6-(3-oxa-7,9-diazabicyclo[3.3.1]nonan-9-ylmethyl)morpholin-4-yl]pyrazolo[1,5-a]pyridine-7-carbonitrile

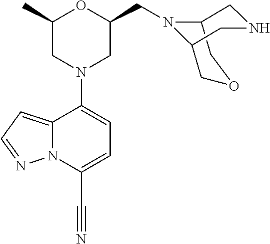 [SMILES: C[C@@H]1CN(c2ccc(C#N)n3nccc23)C[C@H](CN2C3CNCC2COC3)O1]

The title compound was prepared in analogy to the preparation of Example 3 by using tert-butyl 3-oxa-7,9-diazabicyclo[3.3.1]nonane-7-carboxylate instead of tert-butyl 3,9-diazaspiro[5.5]undecane-3-carboxylate. Example 57 (14 mg) was obtained as a light brown semisolid. MS: calc'd 383 (MH⁺), measured 383 (MH⁺). ¹H NMR (400 MHz, METHANOL-d4) δ 8.03 (d, J=2.4 Hz, 1H), 7.47 (d, J=8.1 Hz, 1H), 6.85 (d, J=2.3 Hz, 1H), 6.59 (d, J=8.1 Hz, 1H), 4.13-4.05 (m, 2H), 4.01-3.86 (m, 5H), 3.73 (br d, J=12.2 Hz, 1H), 3.58-3.48 (m, 2H), 3.37 (s, 1H), 3.35-3.32 (m, 1H), 3.13 (dd, J=5.4, 13.6 Hz, 1H), 3.01 (br d, J=13.8 Hz, 2H), 2.86 (dd, J=5.4, 13.6 Hz, 1H), 2.78 (dd, J=10.5, 12.0 Hz, 1H), 2.66 (dd, J=10.4, 12.2 Hz, 1H), 1.24 (d, J=6.2 Hz, 3H).

### Example 58

### 4-[(2S,6R)-2-[(7-amino-3-oxa-9-azabicyclo[3.3.1]nonan-9-yl)methyl]-6-methyl-morpholin-4-yl]pyrazolo[1,5-a]pyridine-7-carbonitrile

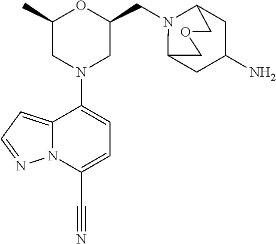 [SMILES: C[C@@H]1CN(c2ccc(C#N)n3nccc23)C[C@H](CN2C3COCC2CC(N)C3)O1]

The title compound was prepared in analogy to the preparation of Example 3 by using tert-butyl N-(3-oxa-9-azabicyclo[3.3.1]nonan-7-yl)carbamate (CAS: 198211-13-1, PharmaBlock) instead of tert-butyl 3,9-diazaspiro[5.5]undecane-3-carboxylate. Example 58 (16 mg) was obtained as a light yellow powder. MS: calc'd 397 (MH⁺), measured 397 (MH⁺). ¹H NMR (400 MHz, METHANOL-d4) δ 8.03 (d, J=2.3 Hz, 1H), 7.47 (d, J=8.1 Hz, 1H), 6.84 (d, J=2.4 Hz, 1H), 6.58 (d, J=7.9 Hz, 1H), 4.35-4.24 (m, 1H), 3.97 (br d, J=12.3 Hz, 1H), 3.94-3.85 (m, 4H), 3.84-3.76 (m, 2H), 3.71 (br d, J=12.2 Hz, 1H), 3.10-3.02 (m, 2H), 2.96 (br s, 1H), 2.73 (dd, J=10.5, 12.3 Hz, 1H), 2.70-2.62 (m, 2H), 2.09-1.99 (m, 1H), 1.98-1.90 (m, 3H), 1.24 (d, J=6.2 Hz, 3H).

### Example 59

### 4-[(2S,6R)-2-[(2-benzylpiperazin-1-yl)methyl]-6-methyl-morpholin-4-yl]pyrazolo[1,5-a]pyridine-7-carbonitrile

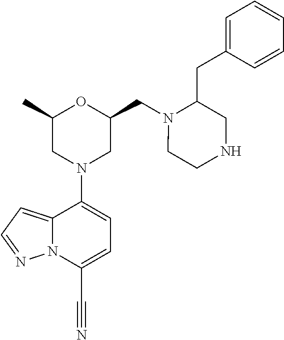 [SMILES: C[C@@H]1CN(c2ccc(C#N)n3nccc23)C[C@H](CN2CCNCC2Cc2ccccc2)O1]

The title compound was prepared in analogy to the preparation of Example 3 by using tert-butyl 3-benzylpiperazine-1-carboxylate (CAS: 502649-29-8, Bepharm) instead of tert-butyl 3,9-diazaspiro[5.5]undecane-3-carboxylate. Example 59 (15 mg) was obtained as a white powder. MS: calc'd 431 (MH⁺), measured 431 (MH⁺). ¹H NMR (400 MHz, METHANOL-d4) δ 8.06 (d, J=2.3 Hz, 1H), 7.50 (dd, J=1.5, 7.9 Hz, 1H), 7.37-7.20 (m, 5H), 6.88 (dd, J=2.4, 5.3 Hz, 1H), 6.60 (t, J=8.4 Hz, 1H), 4.18-3.91 (m, 2H), 3.83-3.69 (m, 2H), 3.52-3.36 (m, 2H), 3.31-3.11 (m, 4H), 3.11-2.99 (m, 2H), 2.99-2.81 (m, 2H), 2.79-2.56 (m, 3H), 1.31 (dd, J=6.2, 12.2 Hz, 3H).

### Example 60

### 4-[(2R,6S)-2-methyl-6-(1-oxa-4,9-diazaspiro[5.5]undecan-9-ylmethyl)morpholin-4-yl]pyrazolo[1,5-a]pyridine-7-carbonitrile

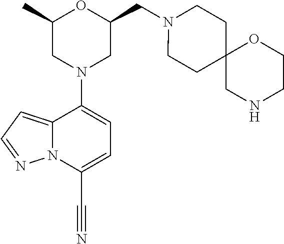 [SMILES: C[C@@H]1CN(c2ccc(C#N)n3nccc23)C[C@H](CN2CCC3(CC2)CNCCO3)O1]

The title compound was prepared in analogy to the preparation of Example 3 by using tert-butyl 1-oxa-4,9-diazaspiro[5.5]undecane-4-carboxylate (CAS: 1023595-11-0, PharmaBlock) instead of tert-butyl 3,9-diazaspiro[5.5]undecane-3-carboxylate. Example 60 (18 mg) was obtained as a light yellow solid. MS: calc'd 411 (MH⁺), measured 411 (MH⁺). ¹H NMR (400 MHz, METHANOL-d4) δ 8.03 (d, J=2.4 Hz, 1H), 7.48 (d, J=7.9 Hz, 1H), 6.90 (d, J=2.4 Hz, 1H), 6.62 (d, J=8.1 Hz, 1H), 4.33 (br t, J=9.5 Hz, 1H), 4.08-3.99 (m, 1H), 3.99-3.91 (m, 2H), 3.85-3.72 (m, 2H), 3.68-3.52 (m, 2H), 3.44-3.32 (m, 3H), 3.30-3.13 (m, 5H), 2.79-2.66 (m, 2H), 2.34 (br d, J=10.0 Hz, 2H), 2.22-1.87 (m, 2H), 1.31 (d, J=6.2 Hz, 3H).

### Example 61

### 4-[(2S,6R)-2-(2,3,4,4a,5,6,7,7a-octahydropyrrolo[3,4-b]pyridin-1-ylmethyl)-6-methyl-morpholin-4-yl]pyrazolo[1,5-a]pyridine-7-carbonitrile

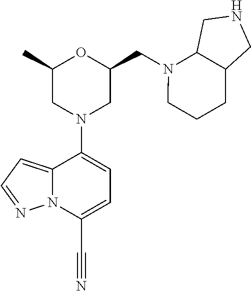 [SMILES: C[C@@H]1CN(c2ccc(C#N)n3nccc23)C[C@H](CN2CCCC3CNCC32)O1]

The title compound was prepared in analogy to the preparation of Example 3 by using tert-butyl 1,2,3,4,4a,5,7,7a-octahydropyrrolo[3,4-b]pyridine-6-carboxylate (CAS: 186203-81-6, PharmaBlock) instead of tert-butyl 3,9-diazaspiro[5.5]undecane-3-carboxylate. Example 61 (19 mg) was obtained as a light yellow powder. MS: calc'd 381 (MH⁺), measured 381 (MH⁺). ¹H NMR (400 MHz, METHANOL-d4) δ 8.03 (dd, J=1.1, 2.2 Hz, 1H), 7.48 (d, J=7.9 Hz, 1H), 6.89 (dd, J=2.4, 4.4 Hz, 1H), 6.62 (dd, J=1.5, 8.0 Hz, 1H), 4.30-4.13 (m, 1H), 4.05-3.93 (m, 1H), 3.93-3.84 (m, 1H), 3.84-3.68 (m, 3H), 3.63-3.50 (m, 1H), 3.49-3.36 (m, 3H), 3.27-2.90 (m, 3H), 2.80-2.63 (m, 3H), 1.99-1.70 (m, 4H), 1.29 (dd, J=2.6, 6.2 Hz, 3H).

### Example 62

### 4-[(2R,6S)-2-methyl-6-[[cis-(3aR,6aR)-3,3a,4,5,6,6a-hexahydro-2H-pyrrolo[3,4-b]pyrrol-1-yl]methyl]morpholin-4-yl]pyrazolo[1,5-a]pyridine-7-carbonitrile

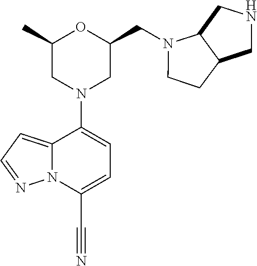

The title compound was prepared in analogy to the preparation of Example 3 by using tert-butyl cis-(3aR,6aR)-2,3,3a,4,6,6a-hexahydro-1H-pyrrolo[3,4-b]pyrrole-5-carboxylate (CAS: 370882-39-6, Accela) instead of tert-butyl 3,9-diazaspiro[5.5]undecane-3-carboxylate. Example 62 (23 mg) was obtained as a light yellow powder. MS: calc'd 367 (MH⁺), measured 367 (MH⁺). ¹H NMR (400 MHz, METHANOL-d4) δ 7.91 (t, J=2.9 Hz, 1H), 7.33 (dd, J=1.2, 7.9 Hz, 1H), 6.77 (t, J=2.4 Hz, 1H), 6.49 (d, J=8.1 Hz, 1H), 4.41 (br s, 1H), 4.25-4.10 (m, 1H), 3.97-3.55 (m, 6H), 3.52-3.23 (m, 6H), 2.70-2.56 (m, 2H), 2.45-2.32 (m, 1H), 1.98-1.77 (m, 1H), 1.20 (t, J=5.8 Hz, 3H).

### Example 63

### 4-[(2R,6S)-2-methyl-6-[[cis-(3aS,7aS)-1,2,3,3a,5,6,7,7a-octahydropyrrolo[3,2-b]pyridin-4-yl]methyl]morpholin-4-yl]pyrazolo[1,5-a]pyridine-7-carbonitrile

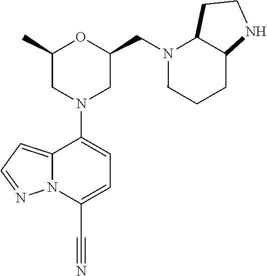

The title compound was prepared in analogy to the preparation of Example 3 by using tert-butyl cis-(3aS,7aS)-2,3,3a,4,5,6,7,7a-octahydropyrrolo[3,2-b]pyridine-1-carboxylate (CAS: 1251010-63-5, PharmaBlock) instead of tert-butyl 3,9-diazaspiro[5.5]undecane-3-carboxylate. Example 63 (13 mg) was obtained as a light yellow semisolid. MS: calc'd 381 (MH⁺), measured 381 (MH⁺). ¹H NMR (400 MHz, METHANOL-d4) δ 8.02 (d, J=2.3 Hz, 1H), 7.46 (d, J=7.9 Hz, 1H), 6.89 (d, J=2.4 Hz, 1H), 6.61 (d, J=8.0 Hz, 1H), 4.31-4.16 (m, 1H), 4.05-3.82 (m, 3H), 3.82-3.72 (m, 2H), 3.70-3.54 (m, 1H), 3.50-3.34 (m, 2H), 3.29-3.18 (m, 1H), 3.17-2.98 (m, 2H), 2.79-2.64 (m, 2H), 2.60-2.47 (m, 1H), 2.45-2.31 (m, 1H), 2.12-1.79 (m, 4H), 1.28 (dd, J=2.2, 6.2 Hz, 3H).

### Example 64

### 4-[(2S,6R)-2-(3,4,4a,5,6,7,8,8a-octahydro-2H-1,5-naphthyridin-1-ylmethyl)-6-methyl-morpholin-4-yl]pyrazolo[1,5-a]pyridine-7-carbonitrile

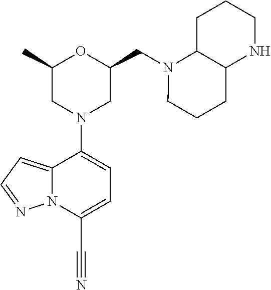 [SMILES: C[C@@H]1CN(c2ccc(C#N)n3nccc23)C[C@H](CN2CCCC3NCCCC32)O1]

The title compound was prepared in analogy to the preparation of Example 3 by using tert-butyl 3,4,4a,5,6,7,8,8a-octahydro-2H-1,5-naphthyridine-1-carboxylate (CAS: 1000931-58-7, PharmaBlock) instead of tert-butyl 3,9-diazaspiro[5.5]undecane-3-carboxylate. Example 64 (13 mg) was obtained as a light yellow powder. MS: calc'd 395 (MH⁺), measured 395 (MH⁺). 11H NMR (400 MHz, METHANOL-d4) δ 8.04 (d, J=2.3 Hz, 1H), 7.48 (d, J=7.9 Hz, 1H), 6.89 (dd, J=2.4, 5.5 Hz, 1H), 6.62 (d, J=7.9 Hz, 1H), 4.37-4.17 (m, 1H), 4.06-3.67 (m, 4H), 3.39-3.32 (m, 2H), 3.30-3.15 (m, 5H), 2.77-2.64 (m, 2H), 2.39-1.85 (m, 7H), 1.84-1.69 (m, 1H), 1.29 (t, J=5.6 Hz, 3H).

### Example 65

### Endo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile

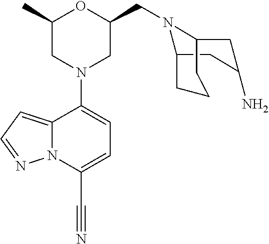 [SMILES: C[C@@H]1CN(c2ccc(C#N)n3nccc23)C[C@H](CN2C3CCCC2CC(N)C3)O1]

The title compound was prepared in analogy to the preparation of Example 3 by using endo-tert-butyl N-(9-azabicyclo[3.3.1]nonan-3-yl)carbamate (CAS: 155560-04-6, PharmaBlock) instead of tert-butyl 3,9-diazaspiro[5.5]undecane-3-carboxylate. Example 65 (25 mg) was obtained as a light yellow powder. MS: calc'd 395 (MH⁺), measured 395 (MH⁺). ¹H NMR (400 MHz, METHANOL-d4) δ 7.93 (d, J=2.4 Hz, 1H), 7.38 (d, J=7.9 Hz, 1H), 6.78 (d, J=2.4 Hz, 1H), 6.52 (d, J=8.1 Hz, 1H), 4.21 (br t, J=9.5 Hz, 1H), 4.03-3.94 (m, 1H), 3.93-3.68 (m, 4H), 3.64 (br d, J=12.5 Hz, 1H), 3.54-3.40 (m, 1H), 3.40-3.29 (m, 1H), 2.71-2.56 (m, 4H), 2.26-2.04 (m, 2H), 2.02-1.84 (m, 1H), 1.80-1.60 (m, 3H), 1.60-1.42 (m, 2H), 1.19 (d, J=6.2 Hz, 3H).

### Example 66

### Exo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile

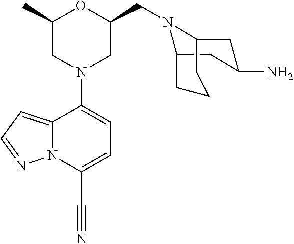 [SMILES: C[C@@H]1CN(c2ccc(C#N)n3nccc23)C[C@H](CN2C3CCCC2CC(N)C3)O1]

The title compound was prepared in analogy to the preparation of Example 3 by using exo-tert-butyl N-(9-azabicyclo[3.3.1]nonan-3-yl)carbamate (CAS: 599165-35-2, PharmaBlock) instead of tert-butyl 3,9-diazaspiro[5.5]undecane-3-carboxylate. Example 66 (24 mg) was obtained as a light yellow powder. MS: calc'd 395 (MH⁺), measured 395 (MH⁺). ¹H NMR (400 MHz, METHANOL-d4) δ 8.03 (d, J=2.4 Hz, 1H), 7.48 (d, J=7.9 Hz, 1H), 6.89 (d, J=2.2 Hz, 1H), 6.63 (d, J=7.9 Hz, 1H), 4.34 (br t, J=9.7 Hz, 1H), 4.27-4.13 (m, 1H), 4.13-3.94 (m, 2H), 3.94-3.80 (m, 2H), 3.80-3.71 (m, 1H), 3.69-3.59 (m, 1H), 3.57-3.50 (m, 1H), 2.82-2.67 (m, 2H), 2.59-2.14 (m, 6H), 2.14-1.99 (m, 1H), 1.99-1.69 (m, 3H), 1.28 (d, J=6.2 Hz, 3H).

### Example 67

### 4-[(2S,6R)-2-(2,9-diazaspiro[5.5]undecan-2-ylmethyl)-6-methyl-morpholin-4-yl]-3-fluoro-pyrazolo[1,5-a]pyridine-7-carbonitrile

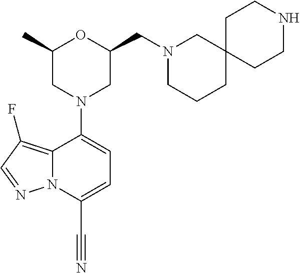 [SMILES: C[C@@H]1CN(c2ccc(C#N)n3ncc(F)c23)C[C@H](CN2CCCC3(CCNCC3)C2)O1]

The title compound was prepared according to the following scheme:

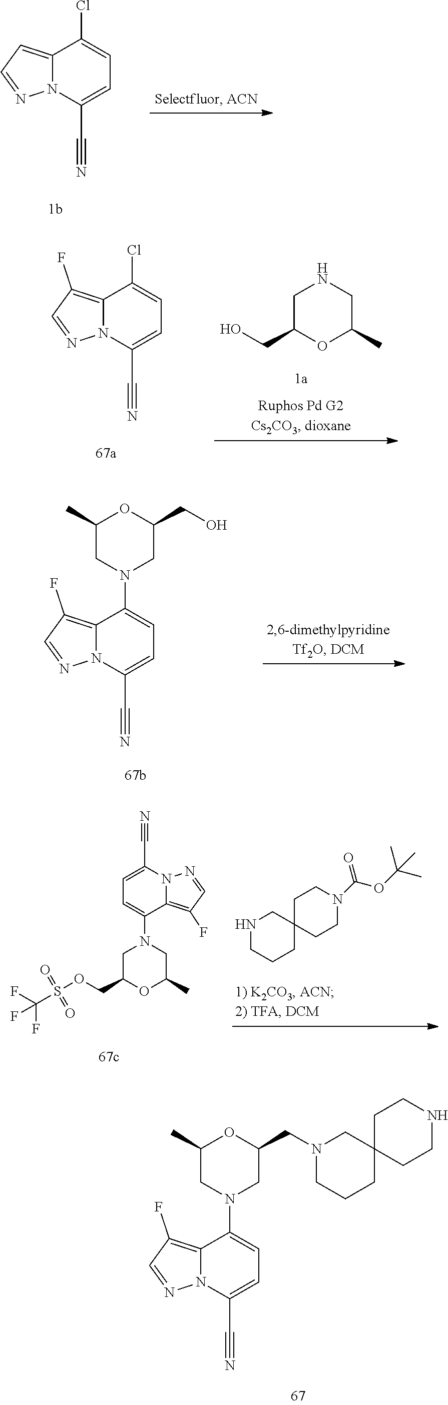

### Step 1: Preparation of 4-chloro-3-fluoro-pyrazolo[1,5-a]pyridine-7-carbonitrile (Compound 67a)

To a solution of 4-chloropyrazolo[1,5-a]pyridine-7-carbonitrile (compound 1b, 600 mg, 3.38 mmol) in ACN (50 mL) was added Selectfluor (2.39 g, 6.76 mmol). After the reaction mixture was stirred at rt for 24 hrs, LCMS indicated the formation of product. The reaction mixture was then concentrated to remove most ACN, diluted with water (30 mL), extracted with DCM. The organic layer was washed with sat. NH₄Cl and brine, dried over Na₂SO₄, and concentrated to give a crude product which was purified by column chromatography to give compound 67a (419 mg) as light yellow powder. MS: calc'd 196 (MH⁺), measured 196 (MH⁺).

### Step 2: Preparation of 3-fluoro-4-[(2R,6R)-2-(hydroxymethyl)-6-methyl-morpholin-4-yl]pyrazolo[1,5-a]pyridine-7-carbonitrile (Compound 67b)

To a solution of 4-chloro-3-fluoropyrazolo[1,5-a]pyridine-7-carbonitrile (compound 67a, 419 mg, 2.14 mmol), (2R,6R)-6-methylmorpholin-2-yl]methanol trifluoroacetate (compound 1a, 526 mg, 2.14 mmol) and Cs₂CO₃ (2.79 g, 8.57 mmol) in dioxane (10 mL) was added RuPhos Pd G2 (116 mg, 0.15 mmol) under N₂. The reaction mixture was heated at 90° C. (oil bath) for 2 h, then cooled to rt, diluted with EtOAc and filtered through celite, the filtrate was concentrated to give a brown oil which was purified by column chromatography to give compound 67b (325 mg) as a yellow oil. MS: calc'd 291 (MH⁺), measured 291 (MH⁺).

### Step 3: Preparation of (2R,6R)-4-(7-cyanopyrazolo[1,5-a]pyridin-4-yl)-6-methyl-morpholin-2-yl]methyl trifluoromethanesulfonate (Compound 67c)

To a solution of 3-fluoro-4-[(2R,6R)-2-(hydroxymethyl)-6-methyl-morpholin-4-yl]pyrazolo[1,5-a]pyridine-7-carbonitrile (compound 67b, 325 mg, 1.12 mmol) in DCM (3 mL) was added 2,6-dimethylpyridine (240 mg, 258 μl, 2.24 mmol) and Tf₂O (474 mg, 284 μl, 1.68 mmol) at rt. After the reaction mixture was stirred at rt for 1.5 hrs, LCMS indicated the formation of product. The mixture was then diluted with DCM, washed with Sat. NH₄Cl and brine. The organic layer was dried over Na₂SO₄ and concentrated to give the crude product which was purified by column chromatography to give compound 67c (180 mg) as a yellow solid. MS: calc'd 423 (MH⁺), measured 423 (MH⁺).

### Step 4: Preparation of 4-[(2S,6R)-2-(2,9-diazaspiro[5.5]undecan-2-ylmethyl)-6-methyl-morpholin-4-yl]-3-fluoro-pyrazolo[1,5-a]pyridine-7-carbonitrile (Example 67)

To a solution of (2R,6R)-4-(7-cyanopyrazolo[1,5-a]pyridin-4-yl)-6-methyl-morpholin-2-yl]methyl trifluoromethanesulfonate (compound 67c, 30 mg, 71 μmol), tert-butyl 2,9-diazaspiro[5.5]undecane-9-carboxylate (27 mg, 0.11 mmol) in ACN (4 mL) was added K₂CO₃ (20 mg, 0.14 mmol). After being stirred at rt for 3 hrs, the reaction mixture was diluted with ACN, and filtered through celite. The filtrate was concentrated to give a yellow solid. The solid was dissolved in DCM (2 mL), and TFA (1 mL) was added dropwise. The reaction mixture was stirred at rt for 1.5 hrs, then concentrated to give a crude product which was purified by prep-HPLC to afford Example 67 (34 mg) as a light yellow powder. MS: calc'd 427 (MH⁺), measured 427 (MH⁺). ¹H NMR (400 MHz, METHANOL-d4) δ 8.02 (d, J=3.5 Hz, 1H), 7.45 (d, J=7.8 Hz, 1H), 6.56 (d, J=7.9 Hz, 1H), 4.39 (br t, J=9.4 Hz, 1H), 4.14-3.98 (m, 1H), 3.81-3.52 (m, 4H), 3.43-3.35 (m, 1H), 3.32-3.23 (m, 5H), 3.14-2.86 (m, 2H), 2.81-2.63 (m, 2H), 2.20-1.62 (m, 7H), 1.57-1.39 (m, 1H), 1.31 (d, J=6.2 Hz, 3H).

### Example 68

### 3-fluoro-4-[(2S,6R)-2-(2,7-diazaspiro[4.5]decan-7-ylmethyl)-6-methyl-morpholin-4-yl]pyrazolo[1,5-a]pyridine-7-carbonitrile

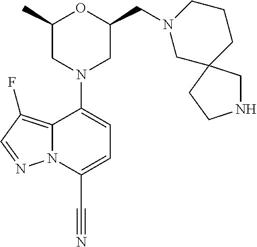 [SMILES: C[C@@H]1CN(c2ccc(C#N)n3ncc(F)c23)C[C@H](CN2CCCC3(CCNC3)C2)O1]

The title compound was prepared in analogy to the preparation of Example 67 by using tert-butyl 2,9-diazaspiro[4.5]decane-2-carboxylate instead of tert-butyl 2,9-diazaspiro[5.5]undecane-9-carboxylate. Example 68 (34 mg) was obtained as a light yellow powder. MS: calc'd 413 (MH⁺), measured 413 (MH⁺). ¹H NMR (400 MHz, METHANOL-d4) δ 8.00 (d, J=3.7 Hz, 1H), 7.43 (d, J=7.9 Hz, 1H), 6.54 (d, J=7.9 Hz, 1H), 4.39-4.27 (m, 1H), 4.10-3.96 (m, 1H), 3.77-3.32 (m, 8H), 3.30-2.92 (m, 4H), 2.80-2.63 (m, 2H), 2.24-1.59 (m, 6H), 1.29 (d, J=6.2 Hz, 3H).

### Example 69

### 3-fluoro-4-[(2S,6R)-2-(2,9-diazaspiro[4.5]decan-2-ylmethyl)-6-methyl-morpholin-4-yl]pyrazolo[1,5-a]pyridine-7-carbonitrile

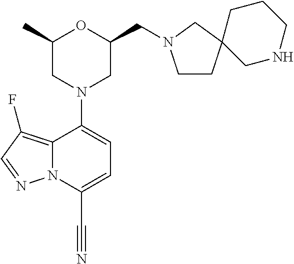 [SMILES: C[C@@H]1CN(c2ccc(C#N)n3ncc(F)c23)C[C@H](CN2CCC3(CCCNC3)C2)O1]

The title compound was prepared in analogy to the preparation of Example 67 by using tert-butyl 2,7-diazaspiro[4.5]decane-7-carboxylate instead of tert-butyl 2,9-diazaspiro[5.5]undecane-9-carboxylate. Example 69 (19 mg) was obtained as a light yellow powder. MS: calc'd 413 (MH⁺), measured 413 (MH⁺). ¹H NMR (400 MHz, METHANOL-d4) δ 8.02 (d, J=3.5 Hz, 1H), 7.45 (d, J=7.8 Hz, 1H), 6.56 (d, J=7.9 Hz, 1H), 4.22 (br t, J=9.8 Hz, 1H), 4.07-3.96 (m, 1H), 3.94-3.77 (m, 1H), 3.65-3.52 (m, 3H), 3.51-3.38 (m, 3H), 3.32-3.18 (m, 4H), 3.18-3.09 (m, 1H), 2.83-2.62 (m, 2H), 2.36-2.00 (m, 2H), 1.99-1.82 (m, 4H), 1.32 (dd, J=1.3, 6.2 Hz, 3H).

### Example 70

### 4-[(2S,6R)-2-(3,7-diazabicyclo[4.2.0]octan-3-ylmethyl)-6-methyl-morpholin-4-yl]pyrazolo[1,5-a]pyridine-7-carbonitrile

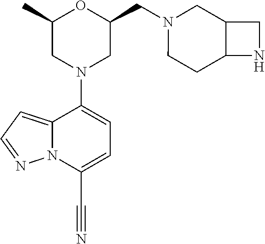 [SMILES: C[C@@H]1CN(c2ccc(C#N)n3nccc23)C[C@H](CN2CCC3NCC3C2)O1]

The title compound was prepared in analogy to the preparation of Example 3 by using tert-butyl 3,7-diazabicyclo[4.2.0]octane-7-carboxylate (CAS: 885271-73-8, PharmaBlock) instead of tert-butyl 3,9-diazaspiro[5.5]undecane-3-carboxylate. Example 70 (19 mg) was obtained as a light yellow powder. MS: calc'd 367 (MH⁺), measured 367 (MH⁺). ¹H NMR (400 MHz, METHANOL-d4) δ 8.06 (d, J=2.3 Hz, 1H), 7.50 (d, J=7.9 Hz, 1H), 6.92 (d, J=2.4 Hz, 1H), 6.65 (d, J=7.9 Hz, 1H), 4.78-4.69 (m, 1H), 4.40-4.31 (m, 1H), 4.29-4.20 (m, 1H), 4.11-3.97 (m, 2H), 3.84 (br d, J=12.2 Hz, 1H), 3.78 (br dd, J=2.0, 12.4 Hz, 1H), 3.74-3.52 (m, 3H), 3.51-3.34 (m, 4H), 2.81-2.69 (m, 2H), 2.57-2.38 (m, 2H), 1.32 (dd, J=1.6, 6.2 Hz, 3H).

### Example 71

### 4-[(2R,6S)-2-methyl-6-(1-oxa-4,9-diazaspiro[5.5]undecan-4-ylmethyl)morpholin-4-yl]pyrazolo[1,5-a]pyridine-7-carbonitrile

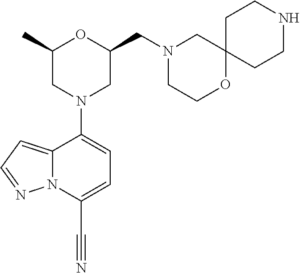 [SMILES: C[C@@H]1CN(c2ccc(C#N)n3nccc23)C[C@H](CN2CCOC3(CCNCC3)C2)O1]

The title compound was prepared in analogy to the preparation of Example 3 by using tert-butyl 1-oxa-4,9-diazaspiro[5.5]undecane-9-carboxylate (CAS: 930785-40-3, PharmaBlock) instead of tert-butyl 3,9-diazaspiro[5.5]undecane-3-carboxylate. Example 71 (16 mg) was obtained as a white powder. MS: calc'd 411 (MH⁺), measured 411 (MH⁺). ¹H NMR (400 MHz, METHANOL-d4) δ 8.06 (d, J=2.4 Hz, 1H), 7.50 (d, J=8.1 Hz, 1H), 6.89 (d, J=2.4 Hz, 1H), 6.64 (d, J=8.1 Hz, 1H), 4.38-4.30 (m, 1H), 4.08-3.96 (m, 3H), 3.83 (br d, J=12.2 Hz, 1H), 3.77 (br d, J=12.5 Hz, 1H), 3.49-3.34 (m, 2H), 3.32-3.16 (m, 8H), 2.80-2.69 (m, 2H), 2.47 (br s, 1H), 2.22 (br s, 1H), 1.95-1.76 (m, 2H), 1.32 (d, J=6.4 Hz, 3H).

### Example 72

### 1-[[(2S,6R)-4-(7-cyanopyrazolo[1,5-a]pyridin-4-yl)-6-methyl-morpholin-2-yl]methyl]piperidine-3-carboxamide

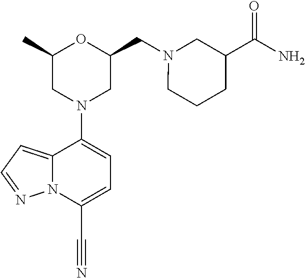 [SMILES: C[C@@H]1CN(c2ccc(C#N)n3nccc23)C[C@H](CN2CCCC(C(N)=O)C2)O1]

The title compound was prepared in analogy to the preparation of Example 1 by using piperidine-3-carboxamide instead of 1-methyl-4-(piperidin-4-yl)piperazine hydrochloride. Example 72 (11 mg) was obtained as a white powder. MS: calc'd 383 (MH⁺), measured 383 (MH⁺). ¹H NMR (400 MHz, METHANOL-d4) δ 8.04 (d, J=2.2 Hz, 1H), 7.48 (d, J=7.9 Hz, 1H), 6.90 (d, J=2.3 Hz, 1H), 6.63 (d, J=7.9 Hz, 1H), 4.39-4.28 (m, 1H), 4.10-3.98 (m, 1H), 3.91-3.50 (m, 4H), 3.41-3.34 (m, 1H), 3.30-3.06 (m, 3H), 3.05-2.94 (m, 1H), 2.81-2.65 (m, 2H), 2.17-1.84 (m, 4H), 1.31 (d, J=6.2 Hz, 3H).

### Example 73

### 4-[(2S,6R)-2-[(4-amino-4-phenyl-1-piperidyl)methyl]-6-methyl-morpholin-4-yl]pyrazolo[1,5-a]pyridine-7-carbonitrile

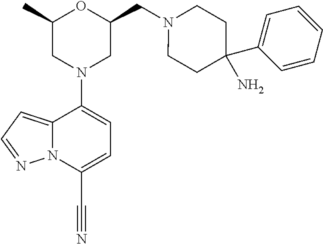 [SMILES: C[C@@H]1CN(c2ccc(C#N)n3nccc23)C[C@H](CN2CCC(N)(c3ccccc3)CC2)O1]

The title compound was prepared in analogy to the preparation of Example 3 by using tert-butyl N-(4-phenyl-4-piperidyl)carbamate instead of tert-butyl 3,9-diazaspiro[5.5]undecane-3-carboxylate. Example 73 (24 mg) was obtained as a white powder. MS: calc'd 431 (MH⁺), measured 431 (MH⁺). ¹H NMR (400 MHz, METHANOL-d4) δ 8.02 (d, J=2.3 Hz, 1H), 7.73-7.68 (m, 2H), 7.62 (t, J=7.5 Hz, 2H), 7.58-7.53 (m, 1H), 7.47 (d, J=8.1 Hz, 1H), 6.86 (d, J=2.4 Hz, 1H), 6.60 (d, J=8.1 Hz, 1H), 4.27 (br t, J=10.3 Hz, 1H), 4.02-3.91 (m, 1H), 3.84-3.68 (m, 4H), 3.30-3.21 (m, 2H), 3.20-2.91 (m, 4H), 2.68 (br t, J=11.4 Hz, 2H), 2.63-2.48 (m, 2H), 1.26 (d, J=6.2 Hz, 3H).

### Example 74

### 4-[(2S,6R)-2-[[(3R,4S)-4-amino-3-methyl-1-piperidyl]methyl]-6-methyl-morpholin-4-yl]pyrazolo[1,5-a]pyridine-7-carbonitrile

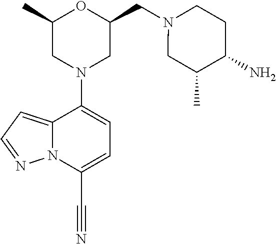 [SMILES: C[C@@H]1CN(c2ccc(C#N)n3nccc23)C[C@H](CN2CC[C@H](N)[C@H](C)C2)O1]

The title compound was prepared in analogy to the preparation of Example 3 by using tert-butyl N-[(3R,4S)-3-methyl-4-piperidyl]carbamate (CAS: 473839-06-4, PharmaBlock) instead of tert-butyl 3,9-diazaspiro[5.5]undecane-3-carboxylate. Example 74 (16 mg) was obtained as a white powder. MS: calc'd 369 (MH⁺), measured 369 (MH⁺). ¹H NMR (400 MHz, METHANOL-d4) δ 8.06 (dd, J=1.2, 2.3 Hz, 1H), 7.50 (d, J=7.9 Hz, 1H), 6.91 (d, J=2.4 Hz, 1H), 6.65 (dd, J=0.8, 8.0 Hz, 1H), 4.42-4.30 (m, 1H), 4.11-4.00 (m, 1H), 3.86-3.75 (m, 2H), 3.75-3.61 (m, 2H), 3.52-3.34 (m, 4H), 3.32-3.18 (m, 1H), 2.80-2.69 (m, 2H), 2.65-2.47 (m, 1H), 2.43-2.10 (m, 2H), 1.33 (dd, J=3.8, 5.7 Hz, 3H), 1.23 (br s, 3H).

### Example 75

### 4-[(2S,6R)-2-[[4-(dimethylamino)-3,3a,4,5,6,6a-hexahydro-1H-cyclopenta[c]pyrrol-2-yl]methyl]-6-methyl-morpholin-4-yl]pyrazolo[1,5-a]pyridine-7-carbonitrile (Racemic Mixture at the * Indicated Positions)

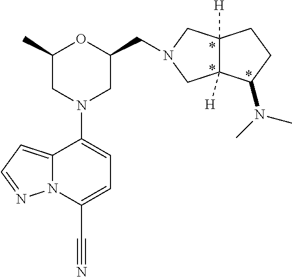 [SMILES: C[C@@H]1CN(c2ccc(C#N)n3nccc23)C[C@H](CN2C[C@H]3CC[C@@H](N(C)C)[C@H]3C2)O1]

The title compound was prepared according to the following scheme:

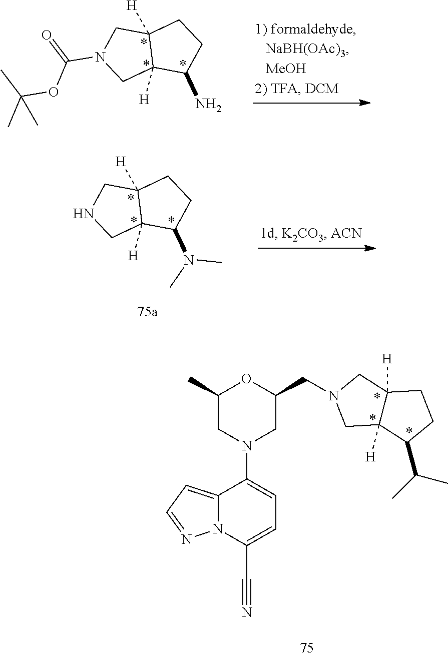 [SMILES: CC(C)(C)OC(=O)N1C[C@H]2CC[C@@H](N)[C@H]2C1 ; CN(C)[C@@H]1CC[C@@H]2CNC[C@@H]21 ; CC(C)[C@@H]1CC[C@@H]2CN(C[C@H]3CN(c4ccc(C#N)n5nccc45)C[C@@H](C)O3)C[C@@H]21]

### Step 1: Preparation of (3aR,4R,6aS)—N,N-dimethyl-1,2,3,3a,4,5,6,6a-octahydrocyclopenta[c]pyrrol-4-amine (Compound 75a)

To a solution of formaldehyde (37% aqueous solution, 1.09 g, 1 mL, 13.4 mmol) and rac-tert-butyl (3aR,4R,6aS)-4-amino-3,3a,4,5,6,6a-hexahydro-1H-cyclopenta[c]pyrrole-2-carboxylate (CAS: 1251012-14-2, Wuxi AppTech) (350 mg, 1.55 mmol) in MeOH (20 mL) was added NaBH(OAc)₃ (1.97 g, 9.28 mmol). The reaction mixture was then stirred at 50° C. for 4 hrs. The reaction mixture was concentrated, poured into sat. NaHCO₃, and extracted with EtOAc. The organic layer was washed with brine, dried over Na₂SO₄ and concentrated to give a crude product (388 mg). To a solution of this crude product (20 mg, 0.080 mmol) in DCM (2 mL) was added TFA (1 mL). The reaction mixture was stirred at rt for 1 h, then concentrated to give a crude compound 75a (24 mg) which was directly used in next step. MS: calc'd 155 (MH⁺), measured 155 (MH⁺).

### Step 2: Preparation of 4-[(2S,6R)-2-[[4-(dimethylamino)-3,3a,4,5,6,6a-hexahydro-1H-cyclopenta[c]pyrrol-2-yl]methyl]-6-methyl-morpholin-4-yl]pyrazolo[1,5-a]pyridine-7-carbonitrile (Example 75)

The title compound was prepared in analogy to the preparation of Example 3 by using (3aR,4R,6aS)—N,N-dimethyl-1,2,3,3a,4,5,6,6a-octahydrocyclopenta[c]pyrrol-4-amine (compound 75a) instead of tert-butyl 3,9-diazaspiro[5.5]undecane-3-carboxylate. Example 75 (12 mg) was obtained as a light yellow oil. MS: calc'd 409 (MH⁺), measured 409 (MH⁺). ¹H NMR (400 MHz, METHANOL-d4) δ 8.04 (dd, J=2.4, 4.0 Hz, 1H), 7.51-7.45 (m, 1H), 6.90 (t, J=2.6 Hz, 1H), 6.63 (d, J=8.1 Hz, 1H), 4.26 (br t, J=10.0 Hz, 1H), 4.12-3.95 (m, 3H), 3.87-3.74 (m, 2H), 3.70 (td, J=5.9, 12.1 Hz, 1H), 3.57-3.38 (m, 3H), 3.28-3.07 (m, 2H), 3.02 (d, J=3.2 Hz, 6H), 3.00-2.90 (m, 1H), 2.79-2.66 (m, 2H), 2.29 (td, J=5.9, 11.9 Hz, 1H), 2.18-2.04 (m, 1H), 2.02-1.88 (m, 1H), 1.86-1.75 (m, 1H), 1.33 (d, J=6.2 Hz, 3H).

### Example 76

### 4-[(2R,6S)-2-methyl-6-[(9-methyl-6-oxa-2,9-diazaspiro[4.5]decan-2-yl)methyl]morpholin-4-yl]pyrazolo[1,5-a]pyridine-7-carbonitrile

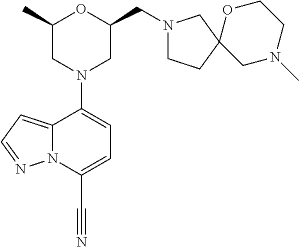 [SMILES: C[C@@H]1CN(c2ccc(C#N)n3nccc23)C[C@H](CN2CCC3(CN(C)CCO3)C2)O1]

The title compound was prepared in analogy to the preparation of Example 1 by using 9-methyl-6-oxa-2,9-diazaspiro[4.5]decane (CAS: 151097-02-8, PharmaBlock) instead of 1-methyl-4-(piperidin-4-yl)piperazine hydrochloride. Example 76 (11 mg) was obtained as an oil. MS: calc'd 411 (MH⁺), measured 411 (MH⁺). ¹H NMR (400 MHz, METHANOL-d4) δ 8.03 (d, J=2.3 Hz, 1H), 7.47 (d, J=7.9 Hz, 1H), 6.88 (t, J=2.3 Hz, 1H), 6.61 (d, J=8.1 Hz, 1H), 4.29-4.18 (m, 1H), 4.10-3.96 (m, 3H), 3.90-3.59 (m, 5H), 3.58-3.31 (m, 7H), 2.94 (s, 3H), 2.78-2.65 (m, 2H), 2.62-2.14 (m, 2H), 1.32 (dd, J=2.7, 6.2 Hz, 3H).

### Example 77

### 4-[(2S,6R)-2-[[2-benzyl-4-(4-piperidyl)piperazin-1-yl]methyl]-6-methyl-morpholin-4-yl]pyrazolo[1,5-a]pyridine-7-carbonitrile

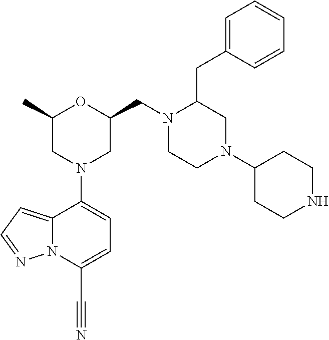 [SMILES: C[C@@H]1CN(c2ccc(C#N)n3nccc23)C[C@H](CN2CCN(C3CCNCC3)CC2Cc2ccccc2)O1]

The title compound was prepared according to the following scheme:

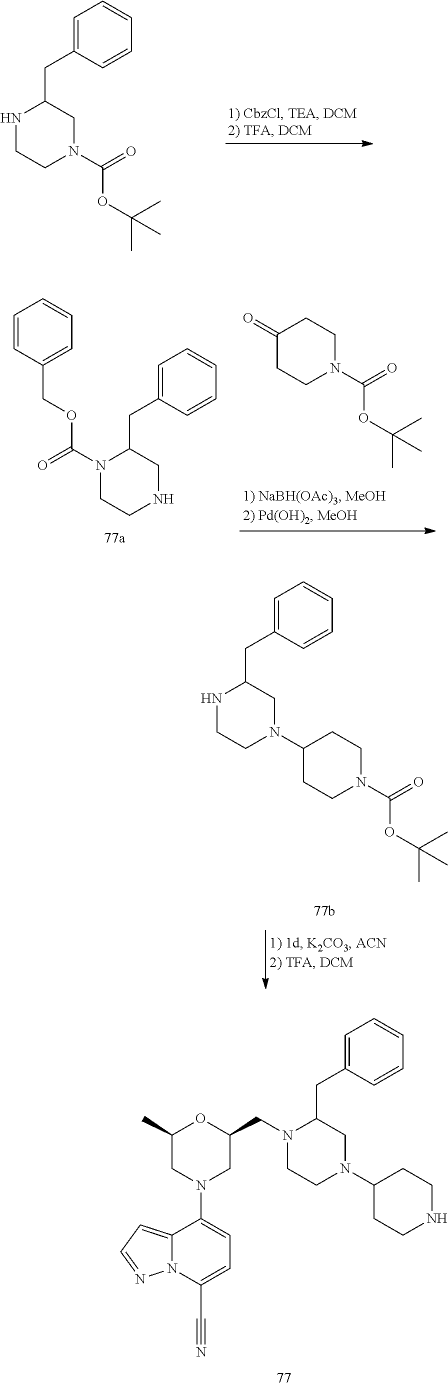 [SMILES: CC(C)(C)OC(=O)N1CCNC(Cc2ccccc2)C1 ; O=C(OCc1ccccc1)N1CCNCC1Cc1ccccc1 ; CC(C)(C)OC(=O)N1CCC(=O)CC1 ; CC(C)(C)OC(=O)N1CCC(N2CCNC(Cc3ccccc3)C2)CC1 ; C[C@@H]1CN(c2ccc(C#N)n3nccc23)C[C@H](CN2CCN(C3CCNCC3)CC2Cc2ccccc2)O1]

### Step 1: Preparation of Benzyl 2-benzylpiperazine-1-carboxylate (Compound 77a)

To a solution of tert-butyl 3-benzylpiperazine-1-carboxylate (300 mg, 1.09 mmol) in DCM (10 mL) was added TEA (330 mg, 0.45 mL, 3.26 mmol) and CbzCl (278 mg, 0.23 mL, 1.63 mmol). After the reaction mixture was then stirred at rt for 2 hrs. The mixture was diluted with water, and extracted with EtOAc. The organic layer was washed with brine, dried over Na₂SO₄ and concentrated to give crude product which was purified by column chromatography to give a product (368 mg) as an oil. The oil was dissolved in DCM (2 ML), then TFA (124 mg, 84 μL, 1.09 mmol) was added. The reaction mixture was stirred at rt overnight, then concentrated to give a crude compound 77a (385 mg) as an oil which is directly used in next step. MS: calc'd 311 (MH⁺), measured 311 (MH⁺).

### Step 2: Preparation of tert-butyl 4-(3-benzylpiperazin-1-yl)piperidine-1-carboxylate (Compound 77b)

To a solution of benzyl 2-benzylpiperazine-1-carboxylate (compound 77a, 84 mg, 0.27 mmol) and tert-butyl 4-oxopiperidine-1-carboxylate (65 mg, 0.32 mmol) in ACN/MeOH (5 mL, v/v=4/1). The reaction mixture was stirred at 80° C. for 24 hrs, another portion of tert-butyl 4-oxopiperidine-1-carboxylate (65 mg, 0.32 mmol) and NaBH(OAc)₃ (114 mg, 0.54 mmol) were added. The reaction mixture was stirred at 80° C. for 2 d, then cooled to rt, diluted with water and extracted with EtOAc. The organic layer was washed with brine, dried over Na₂SO₄ and concentrated to give a crude product which was purified by column chromatography to give a pure product (264 mg) as an oil. The oil was then dissolved in MeOH (4 mL), Pd(OH)₂/C (26 mg, 20% wet) was added. The reaction mixture was charged with H₂ balloon and stirred at rt for 2 hrs, then filtered through celite. The filtrate was concentrated to give crude compound 77b (100 mg) as an oil which was directly used in next step. MS: calc'd 360 (MH⁺), measured 360 (MH⁺).

### Step 3: Preparation of 4-[(2S,6R)-2-[[2-benzyl-4-(4-piperidyl)piperazin-1-yl]methyl]-6-methyl-morpholin-4-yl]pyrazolo[1,5-a]pyridine-7-carbonitrile (Example 77)

The title compound was prepared in analogy to the preparation of Example 3 by using tert-butyl 4-(3-benzylpiperazin-1-yl)piperidine-1-carboxylate (compound 77b) instead of tert-butyl 3,9-diazaspiro[5.5]undecane-3-carboxylate. Example 77 (6 mg) was obtained as a light yellow solid. MS: calc'd 514 (MH⁺), measured 514 (MH⁺). ¹H NMR (400 MHz, METHANOL-d4) δ 8.07 (s, 1H), 7.52 (d, J=7.8 Hz, 1H), 7.43-7.36 (m, 2H), 7.35-7.27 (m, 3H), 6.93 (dd, J=2.4, 4.3 Hz, 1H), 6.66 (dd, J=4.0, 8.1 Hz, 1H), 4.40-4.28 (m, 1H), 4.12-4.01 (m, 1H), 3.90-3.77 (m, 3H), 3.76-3.58 (m, 2H), 3.53-3.37 (m, 5H), 3.15-2.91 (m, 5H), 2.90-2.59 (m, 5H), 2.11-1.99 (m, 2H), 1.65 (br d, J=10.6 Hz, 2H), 1.36 (dd, J=3.1, 6.1 Hz, 3H).

### Example 81

The following tests were carried out in order to determine the activity of the compounds of formula (I) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signalling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlr1-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotorneter. The signalling pathway that TLR9 activation leads to downstream NF-κB activation has been widely accepted, and therefore similar reporter assay was modified for evaluating TLR9 antagonist.

The compounds of formula (I) have human TLR7 and/or TLR8 inhibitory activities (IC₅₀ value)<1 μM, particularly <0.1 μM. Moreover, some compounds also have human TLR9 inhibitory activity <1 μM, particularly <0.3 μM. Activity data of the compounds of the present invention were shown in Table 1.

TABLE 1
The activity of the compounds of present invention
in HEK293-Blue-hTLR-7/8/9 cells assays

| Example | TLR7 IC₅₀ (μM) | TLR8 IC₅₀ (μM) | TLR9 IC₅₀ (μM) |
|---|---|---|---|
| 1 | 0.095 | 0.028 | 0.13 |
| 3 | 0.24 | 0.62 | 0.090 |
| 4 | 0.18 | 0.026 | 0.28 |
| 5 | 0.054 | 0.078 | 0.89 |
| 6 | 0.20 | 0.14 | 0.031 |
| 7 | 0.26 | 0.32 | 0.32 |
| 10 | 0.083 | 0.13 | 0.080 |
| 11 | 0.16 | 0.93 | 0.097 |
| 12 | 0.24 | 0.28 | 0.20 |
| 13 | 0.40 | 0.74 | 0.27 |
| 14 | 0.46 | 0.38 | 3.0 |
| 15 | 0.077 | 0.18 | 23 |
| 16 | 0.10 | 0.019 | 0.52 |
| 17 | 0.089 | 0.058 | 0.18 |
| 18 | 0.20 | 0.35 | 0.089 |
| 19 | 0.13 | 0.25 | 0.092 |
| 20 | 0.65 | 0.61 | 12 |
| 21 | 0.098 | 0.039 | 16 |
| 22 | 0.10 | 0.069 | 0.18 |
| 23 | 0.18 | 0.041 | 0.25 |
| 24 | 0.10 | 0.61 | 0.10 |
| 25 | 0.11 | 0.088 | 0.088 |
| 27 | 0.13 | 0.19 | 0.065 |
| 28 | 0.044 | 0.065 | 14 |
| 29 | 0.076 | 0.052 | 0.22 |
| 30 | 0.046 | 0.10 | 3.2 |
| 31 | 0.053 | 0.097 | 10 |
| 32 | 0.41 | 0.091 | 0.065 |
| 33 | 0.46 | 0.054 | 0.12 |
| 34 | 0.087 | 0.065 | 99 |
| 35 | 0.83 | 0.95 | >100 |
| 36 | 0.038 | 0.090 | 0.52 |
| 37 | 0.19 | 0.045 | 0.078 |
| 38 | 0.12 | 0.037 | 1.9 |
| 39 | 0.20 | 0.18 | 0.096 |
| 40 | 0.095 | 0.14 | 13 |
| 41 | 0.078 | 0.029 | 0.14 |
| 42 | 0.098 | 0.065 | 1.7 |
| 43 | 0.059 | 0.031 | 10 |
| 44 | 0.24 | 0.025 | 0.11 |
| 45 | 0.25 | 0.23 | 0.97 |
| 46 | 0.60 | 0.15 | 0.14 |
| 47 | 0.18 | 0.031 | 0.19 |
| 48 | 0.30 | 0.11 | 0.25 |
| 49 | 0.18 | 0.15 | 0.12 |
| 50A | 0.23 | 0.064 | 16 |
| 50B | 0.14 | 0.052 | 24 |
| 51 | 0.39 | 0.040 | 0.23 |
| 52 | 0.092 | 0.053 | 0.26 |
| 53 | 0.23 | 0.029 | 0.16 |
| 54 | 0.19 | 0.042 | 0.51 |
| 55 | 0.54 | 0.87 | 25 |
| 56 | 0.27 | 0.48 | 0.85 |
| 57 | 0.073 | 0.062 | 10 |
| 58 | 0.090 | 0.058 | 0.39 |
| 59 | 0.007 | 0.006 | 1.5 |
| 60 | 0.10 | 0.096 | 0.18 |
| 61 | 0.46 | 0.30 | 6.1 |
| 62 | 0.71 | 0.26 | 2.4 |
| 63 | 0.27 | 0.044 | 5.9 |
| 64 | 0.25 | 0.12 | 2.2 |
| 65 | 0.46 | 0.064 | 0.14 |
| 66 | 0.49 | 0.14 | 0.12 |
| 67 | 0.28 | 0.038 | 0.087 |
| 68 | 0.23 | 0.011 | 0.11 |
| 69 | 0.13 | 0.026 | 0.13 |
| 70 | 0.23 | 0.12 | 0.20 |
| 71 | 0.45 | 0.055 | 0.51 |
| 73 | 0.15 | 0.047 | 0.22 |
| 74 | 0.089 | 0.039 | 0.22 |
| 75 | 0.68 | 0.60 | 0.29 |
| 76 | 0.92 | 0.34 | 3.5 |
| 77 | 0.16 | 0.13 | 0.15 |

1. A compound of formula (I), 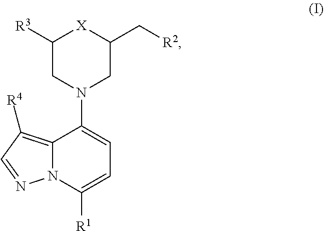
wherein
R¹ is cyano, C₁₋₆alkyl, halogen, haloC₁₋₆alkyl or nitro;
R² is heterocyclyl or heterocyclylamino;
R³ is C₁₋₆alkyl or haloC₁₋₆alkyl;
R⁴ is H or halogen;
X is O or CH₂;
or a pharmaceutically acceptable salt, enantiomer or diastereomer thereof.

2. A compound according to claim 1, wherein
R¹ is cyano;
R² is (3,4,4a,5,6,7,8,8a-octahydro-2H-naphthyridinyl;
(C₁₋₆alkyl)₂amino-3,3a,4,5,6,6a-hexahydro-1H-cyclopenta[c]pyrrolyl;
1,2,3,3a,4,6,7,7a-octahydropyrrolo[3,2-c]pyridinyl;
1,2,3,3a,5,6,7,7a-octahydropyrrolo[3,2-b]pyridinyl;
1,2,3,4,4a,5,7,7a-octahydropyrrolo[3,4-b]pyridinyl;
1,3,3a,4,5,6,7,7a-octahydropyrrolo[3,4-c]pyridinyl;
2,3,3a,4,6,6a-hexahydro-1H-pyrrolo[3,4-c]pyrrolyl;
2,3,4,4a,5,6,7,7a-octahydropyrrolo[3,4-b]pyridinyl;
3,3a,4,5,6,6a-hexahydro-2H-pyrrolo[3,4-b]pyrrolyl;
aminoazabicyclo[3.2.1]octanyl;
aminoazabicyclo[3.3.1]nonanyl;
aminoazaspiro[3.3]heptanyl;
aminooxaazabicyclo[3.3.1]nonanyl;
C₁₋₆alkyloxadiazaspiro[4.5]decanyl;
C₁₋₆alkylpiperidinylamino;
diazabicyclo[2.2.2]octanyl;
diazabicyclo[3.2.1]octanyl;
diazabicyclo[4.2.0]octanyl;
diazaspiro[2.5]octanyl;
diazaspiro[3.3]heptanyl;
diazaspiro[3.4]octanyl;
diazaspiro[3.5]nonanyl;
diazaspiro[3.6]decanyl;
diazaspiro[4.4]nonanyl;
diazaspiro[4.5]decanyl;
diazaspiro[5.5]undecanyl;
oxadiazabicyclo[3.3.1]nonanyl;
oxadiazaspiro[5.5]undecanyl;
oxodiazaspiro[4.4]nonanyl;
piperazinyl, said piperazinyl being unsubstituted or substituted by one, two or three substituents independently selected from C₁₋₆alkyl, phenyl, phenylC₁₋₆alkyl, aminoC₁₋₆alkylcarbonyl and piperidinyl;
piperidinyl, said piperidinyl being unsubstituted or substituted by one, two or three substituents independently selected from amino, aminoC₁₋₆alkyl, azepanyl, C₁₋₆alkyl, C₁₋₆alkylcarbonylamino, C₁₋₆alkylpiperazinyl, carbamoyl, halogen, phenyl, piperazinyl, piperidinyl and pyrrolidinyl; or
pyrrolidinyl, said pyrrolidinyl being unsubstituted or substituted by one, two or three substituents independently selected from amino and halogen;
R³ is C₁₋₆alkyl;
R⁴ is H or halogen;
X is O;
or a pharmaceutically acceptable salt, enantiomer or diastereomer thereof.

3. A compound according to claim 2, wherein
R¹ is cyano;
R² is (3,4,4a,5,6,7,8,8a-octahydro-2H-naphthyridinyl;
2,2-dimethylpropanoylamino-3,3a,4,5,6,6a-hexahydro-1H-cyclopenta[c]pyrrolyl;
1,2,3,3a,4,6,7,7a-octahydropyrrolo[3,2-c]pyridinyl;
1,2,3,3a,5,6,7,7a-octahydropyrrolo[3,2-b]pyridinyl;
1,2,3,4,4a,5,7,7a-octahydropyrrolo[3,4-b]pyridinyl;
1,3,3a,4,5,6,7,7a-octahydropyrrolo[3,4-c]pyridinyl;
2,3,3a,4,6,6a-hexahydro-1H-pyrrolo[3,4-c]pyrrolyl;
2,3,4,4a,5,6,7,7a-octahydropyrrolo[3,4-b]pyridinyl;
3,3a,4,5,6,6a-hexahydro-2H-pyrrolo[3,4-b]pyrrolyl;
aminoazabicyclo[3.2.1]octanyl;
aminoazabicyclo[3.3.1]nonanyl;
aminoazaspiro[3.3]heptanyl;
aminooxaazabicyclo[3.3.1]nonanyl;
methyloxadiazaspiro[4.5]decanyl;
methylpiperidinylamino;
diazabicyclo[2.2.2]octanyl;
diazabicyclo[3.2.1]octanyl;
diazabicyclo[4.2.0]octanyl;
diazaspiro[2.5]octanyl;
diazaspiro[3.3]heptanyl;
diazaspiro[3.4]octanyl;
diazaspiro[3.5]nonanyl;
diazaspiro[3.6]decanyl;
diazaspiro[4.4]nonanyl;
diazaspiro[4.5]decanyl;
diazaspiro[5.5]undecanyl;
oxadiazabicyclo[3.3.1]nonanyl;
oxadiazaspiro[5.5]undecanyl;
oxodiazaspiro[4.4]nonanyl;
piperazinyl, said piperazinyl being unsubstituted or substituted by one, two or three substituents independently selected from methyl, phenyl, benzyl, aminoacetyl and piperidinyl;
piperidinyl, said piperidinyl being unsubstituted or substituted by one, two or three substituents independently selected from amino, aminomethyl, aminoethyl, azepanyl, methyl, 2,2-dimethylpropanoylamino, methylpiperazinyl, carbamoyl, fluoro, phenyl, piperazinyl, piperidinyl and pyrrolidinyl; or
pyrrolidinyl, said pyrrolidinyl being unsubstituted or substituted by one, two or three substituents independently selected from amino and fluoro;
R³ is methyl;
R⁴ is H or fluoro;
X is O;
or a pharmaceutically acceptable salt, enantiomer or diastereomer thereof.

4. A compound according to claim 3, wherein R² is (3,4,4a,5,6,7,8,8a-octahydro-2H-1,5-naphthyridin-1-yl; 1,2,3,3a,4,6,7,7a-octahydropyrrolo[3,2-c]pyridin-5-yl; 1,2,3,3a,5,6,7,7a-octahydropyrrolo[3,2-b]pyridin-4-yl; 1,2,3,4,4a,5,7,7a-octahydropyrrolo[3,4-b]pyridin-6-yl, 1,3,3a,4,5,6,7,7a-octahydropyrrolo[3,4-c]pyridin-2-yl; 1,6-diazaspiro[3.3]heptan-1-yl; 1,7-diazaspiro[3.5]nonan-7-yl; 1,8-diazaspiro[4.5]decan-8-yl; 1,9-diazaspiro[5.5]undecan-9-yl, 1-methyl-4-piperidinylamino; 1-oxa-4,9-diazaspiro[5.5]undecan-4-yl; 1-oxa-4,9-diazaspiro[5.5]undecan-9-yl; 1-oxo-2,7-diazaspiro[4.4]nonan-2-yl; 2-(3-carbamoyl)-1-piperidinyl; 2,3,3a,4,6,6a-hexahydro-1H-pyrrolo[3,4-c]pyrrol-5-yl; 2,3,4,4a,5,6,7,7a-octahydropyrrolo[3,4-b]pyridin-1-yl; 2,5-diazabicyclo[2.2.2]octan-2-yl; 2,6-diazaspiro[3.3]heptan-2-yl; 2,6-diazaspiro[3.5]nonan-6-yl; 2,6-diazaspiro[4.5]decan-2-yl; 2,7-diazaspiro[3.4]octan-2-yl; 2,7-diazaspiro[3.5]nonan-2-yl; 2,7-diazaspiro[3.5]nonan-7-yl; 2,7-diazaspiro[4.4]nonan-2-yl; 2,7-diazaspiro[4.5]decan-7-yl; 2,8-diazaspiro[3.5]nonan-2-yl; 2,8-diazaspiro[3.6]decan-2-yl; 2,8-diazaspiro[4.5]decan-2-yl; 2,8-diazaspiro[4.5]decan-8-yl; 2,8-diazaspiro[5.5]undecan-2-yl; 2,9-diazaspiro[4.5]decan-2-yl; 2,9-diazaspiro[5.5]undecan-2-yl; 2,9-diazaspiro[5.5]undecan-9-yl; 2-benzyl-4-(4-piperidinyl)piperazin-1-yl; 2-benzylpiperazin-1-yl; 2-methylpiperazin-1-yl; 3,3a,4,5,6,6a-hexahydro-2H-pyrrolo[3,4-b]pyrrol-1-yl; 3,7-diazabicyclo[4.2.0]octan-3-yl; 3,7-diazabicyclo[4.2.0]octan-7-yl; 3,8-diazabicyclo[3.2.1]octan-3-yl; 3,8-diazabicyclo[3.2.1]octan-8-yl; 3,8-diazabicyclo[4.2.0]octan-8-yl; 3,9-diazaspiro[5.5]undecan-3-yl; 3-amino-8-azabicyclo[3.2.1]octan-8-yl; 3-amino-9-azabicyclo[3.3.1]nonan-9-yl; 3-methylpiperazin-1-yl; 3-oxa-7,9-diazabicyclo[3.3.1]nonan-9-yl; 3-oxo-2,7-diazaspiro[4.4]nonan-2-yl; 3-phenylpiperazin-1-yl; 4-(1-piperidinyl)-1-piperidinyl; 4-(2,2-dimethylpropanoylamino)-1-piperidinyl; 4-(2-aminoacetyl)piperazinyl; 4-(2-aminoethyl)-1-piperidinyl; 4-(4-methylpiperazin-1-yl)-1-piperidinyl; 4-(aminomethyl)-1-piperidinyl; 4-(azepan-1-yl)-1-piperidinyl; 4-(dimethylamino)-3,3a,4,5,6,6a-hexahydro-1H-cyclopenta[c]pyrrol-2-yl; 4,7-diazaspiro[2.5]octan-7-yl; 4-amino-1-piperidinyl; 4-amino-2-methyl-1-piperidinyl; 4-amino-3,3-difluoro-1-piperidinyl; 4-amino-3,3-difluoro-pyrrolidin-1-yl; 4-amino-3-methyl-1-piperidinyl; 4-amino-4-methyl-1-piperidinyl; 4-amino-4-phenyl-1-piperidinyl; 4-methylpiperazinyl; 4-piperazin-1-yl-1-piperidinyl; 4-pyrrolidin-1-yl-1-piperidinyl; 6-amino-2-azaspiro[3.3]heptan-2-yl; 7-amino-3-oxa-9-azabicyclo[3.3.1]nonan-9-yl; 8-amino-3-azabicyclo[3.2.1]octan-3-yl; 9-methyl-6-oxa-2,9-diazaspiro[4.5]decan-2-yl or piperazinyl.

5. A compound according to claim 2, wherein
R² is 1,2,3,3a,4,6,7,7a-octahydropyrrolo[3,2-c]pyridinyl;
2,3,3a,4,6,6a-hexahydro-1H-pyrrolo[3,4-c]pyrrolyl;
aminoazabicyclo[3.2.1]octanyl;
aminooxaazabicyclo[3.3.1]nonanyl;
diazabicyclo[2.2.2]octanyl;
diazabicyclo[3.2.1]octanyl;
diazabicyclo[4.2.0]octanyl;
diazaspiro[4.5]decanyl;
oxadiazabicyclo[3.3.1]nonanyl;
oxodiazaspiro[4.4]nonanyl;
piperazinyl, said piperazinyl being unsubstituted or substituted by C₁₋₆alkyl or phenylC₁₋₆alkyl; or
piperidinyl, said piperidinyl being unsubstituted or substituted by one, two or three substituents independently selected from amino, C₁₋₆alkyl, C₁₋₆alkylpiperazinyl and halogen.

6. A compound according to claim 5, wherein R² is 1,2,3,3a,4,6,7,7a-octahydropyrrolo[3,2-c]pyridinyl; 2,3,3a,4,6,6a-hexahydro-1H-pyrrolo[3,4-c]pyrrolyl; aminoazabicyclo[3.2.1]octanyl; aminooxaazabicyclo[3.3.1]nonanyl; diazabicyclo[2.2.2]octanyl; diazabicyclo[3.2.1]octanyl; diazabicyclo[4.2.0]octanyl; diazaspiro[4.5]decanyl; oxadiazabicyclo[3.3.1]nonanyl; oxodiazaspiro[4.4]nonanyl; piperazinyl, methylpiperazinyl; benzylpiperazinyl; methylpiperazinylpiperidinyl; aminopiperidinyl; amino(C₁₋₆alkyl)piperidinyl or aminohalopiperidinyl.

7. A compound according to claim 6, wherein R² is 4-(4-methylpiperazin-1-yl)-1-piperidinyl; 1,2,3,3a,4,6,7,7a-octahydropyrrolo[3,2-c]pyridin-5-yl; 1-oxo-2,7-diazaspiro[4.4]nonan-2-yl; 2,3,3a,4,6,6a-hexahydro-1H-pyrrolo[3,4-c]pyrrol-5-yl; 2,5-diazabicyclo[2.2.2]octan-2-yl; 2,6-diazaspiro[4.5]decan-2-yl; 2-benzylpiperazin-1-yl; 3,7-diazabicyclo[4.2.0]octan-7-yl; 3,8-diazabicyclo[3.2.1]octan-3-yl; 3,8-diazabicyclo[3.2.1]octan-8-yl; 3-methylpiperazin-1-yl; 3-oxa-7,9-diazabicyclo[3.3.1]nonan-9-yl; 4-amino-1-piperidinyl; 4-amino-3,3-difluoro-1-piperidinyl; 4-amino-3-methyl-1-piperidinyl; 4-amino-4-methyl-1-piperidinyl; 7-amino-3-oxa-9-azabicyclo[3.3.1]nonan-9-yl; 8-amino-3-azabicyclo[3.2.1]octan-3-yl or piperazinyl.

8. A compound according to claim 5 or 6, wherein R² is C₁₋₆alkylpiperazinylpiperidinyl; 1,2,3,3a,4,6,7,7a-octahydropyrrolo[3,2-c]pyridinyl; oxadiazaspiro[5.5]undecanyl; 2,3,3a,4,6,6a-hexahydro-1H-pyrrolo[3,4-c]pyrrolyl; diazaspiro[4.5]decanyl; diazabicyclo[4.2.0]octanyl; aminopiperidinyl; amino(C₁₋₆alkyl)piperidinyl.

9. A compound according to claim 8, wherein R² is 4-(4-methylpiperazin-1-yl)-1-piperidinyl; 1,2,3,3a,4,6,7,7a-octahydropyrrolo[3,2-c]pyridin-5-yl; 1-oxa-4,9-diazaspiro[5.5]undecan-9-yl; 2,3,3a,4,6,6a-hexahydro-1H-pyrrolo[3,4-c]pyrrol-5-yl; 2,9-diazaspiro[4.5]decan-2-yl; 3,7-diazabicyclo[4.2.0]octan-7-yl; 4-amino-1-piperidinyl; 4-amino-3-methyl-1-piperidinyl; 4-amino-4-methyl-1-piperidinyl.

10. A compound according to claim 8, wherein R² is diazaspiro[4.5]decanyl.

11. A compound according to claim 10, wherein R² is 2,9-diazaspiro[4.5]decan-2-yl.

12. A compound according to claim 2, selected from:
4-[(2R,6S)-2-methyl-6-[[4-(4-methylpiperazin-1-yl)-1-piperidyl]methyl]morpholin-4-yl]pyrazolo[1,5-a]pyridine-7-carbonitrile;
N-[1-[[(2S,6R)-4-(7-cyanopyrazolo[1,5-a]pyridin-4-yl)-6-methyl-morpholin-2-yl]methyl]-4-piperidyl]-2,2-dimethyl-propanamide;
4-[(2S,6R)-2-(3,9-diazaspiro[5.5]undecan-3-ylmethyl)-6-methyl-morpholin-4-yl]pyrazolo[1,5-a]pyridine-7-carbonitrile;
4-[(2S,6R)-2-[[4-(azepan-1-yl)-1-piperidyl]methyl]-6-methyl-morpholin-4-yl]pyrazolo[1,5-a]pyridine-7-carbonitrile;
4-[(2R,6S)-2-methyl-6-(piperazin-1-ylmethyl)morpholin-4-yl]pyrazolo[1,5-a]pyridine-7-carbonitrile;
4-[(2S,6R)-2-(2,7-diazaspiro[4.4]nonan-2-ylmethyl)-6-methyl-morpholin-4-yl]pyrazolo[1,5-a]pyridine-7-carbonitrile;
4-[(2R,6S)-2-methyl-6-[[(1-methyl-4-piperidyl)amino]methyl]morpholin-4-yl]pyrazolo[1,5-a]pyridine-7-carbonitrile;
4-[(2S,6R)-2-(2,6-diazaspiro[3.3]heptan-2-ylmethyl)-6-methyl-morpholin-4-yl]pyrazolo[1,5-a]pyridine-7-carbonitrile;
4-[(2S,6R)-2-(2,7-diazaspiro[3.4]octan-2-ylmethyl)-6-methyl-morpholin-4-yl]pyrazolo[1,5-a]pyridine-7-carbonitrile;
4-[(2S,6R)-2-(2,8-diazaspiro[3.5]nonan-2-ylmethyl)-6-methyl-morpholin-4-yl]pyrazolo[1,5-a]pyridine-7-carbonitrile;
4-[(2S,6R)-2-[(6-amino-2-azaspiro[3.3]heptan-2-yl)methyl]-6-methyl-morpholin-4-yl]pyrazolo[1,5-a]pyridine-7-carbonitrile;
4-[(2S,6R)-2-[[4-(aminomethyl)-1-piperidyl]methyl]-6-methyl-morpholin-4-yl]pyrazolo[1,5-a]pyridine-7-carbonitrile;
4-[(2S,6R)-2-[[4-(2-aminoethyl)-1-piperidyl]methyl]-6-methyl-morpholin-4-yl]pyrazolo[1,5-a]pyridine-7-carbonitrile;
4-[(2S,6R)-2-[[4-(2-aminoacetyl)piperazin-1-yl]methyl]-6-methyl-morpholin-4-yl]pyrazolo[1,5-a]pyridine-7-carbonitrile;
4-[(2R,6S)-2-methyl-6-[(4-methylpiperazin-1-yl)methyl]morpholin-4-yl]pyrazolo[1,5-a]pyridine-7-carbonitrile;
4-[(2R,6S)-2-methyl-6-[[4-(1-piperidyl)-1-piperidyl]methyl]morpholin-4-yl]pyrazolo[1,5-a]pyridine-7-carbonitrile;
4-[(2S,6R)-2-[(4-amino-1-piperidyl)methyl]-6-methyl-morpholin-4-yl]pyrazolo[1,5-a]pyridine-7-carbonitrile;
4-[(2S,6R)-2-(2,8-diazaspiro[4.5]decan-2-ylmethyl)-6-methyl-morpholin-4-yl]pyrazolo[1,5-a]pyridine-7-carbonitrile;
4-[(2S,6R)-2-(2,8-diazaspiro[4.5]decan-8-ylmethyl)-6-methyl-morpholin-4-yl]pyrazolo[1,5-a]pyridine-7-carbonitrile;
4-[(2R,6R)-2-methyl-6-[(3-oxo-2,7-diazaspiro[4.4]nonan-2-yl)methyl]morpholin-4-yl]pyrazolo[1,5-a]pyridine-7-carbonitrile;
4-[(2R,6R)-2-methyl-6-[(1-oxo-2,7-diazaspiro[4.4]nonan-2-yl)methyl]morpholin-4-yl]pyrazolo[1,5-a]pyridine-7-carbonitrile;
4-[(2S,6R)-2-(2,3,3a,4,6,6a-hexahydro-1H-pyrrolo[3,4-c]pyrrol-5-ylmethyl)-6-methyl-morpholin-4-yl]pyrazolo[1,5-a]pyridine-7-carbonitrile;
4-[(2S,6R)-2-(2,8-diazaspiro[5.5]undecan-2-ylmethyl)-6-methyl-morpholin-4-yl]pyrazolo[1,5-a]pyridine-7-carbonitrile;
4-[(2S,6R)-2-(2,7-diazaspiro[3.5]nonan-2-ylmethyl)-6-methyl-morpholin-4-yl]pyrazolo[1,5-a]pyridine-7-carbonitrile;
4-[(2S,6R)-2-(2,9-diazaspiro[4.5]decan-2-ylmethyl)-6-methyl-morpholin-4-yl]pyrazolo[1,5-a]pyridine-7-carbonitrile;
4-[(2S,6R)-2-(2,7-diazaspiro[3.5]nonan-7-ylmethyl)-6-methyl-morpholin-4-yl]pyrazolo[1,5-a]pyridine-7-carbonitrile;
4-[(2S,6R)-2-(2,9-diazaspiro[5.5]undecan-9-ylmethyl)-6-methyl-morpholin-4-yl]pyrazolo[1,5-a]pyridine-7-carbonitrile;
4-[(2R,6S)-2-methyl-6-[(3-methylpiperazin-1-yl)methyl]morpholin-4-yl]pyrazolo[1,5-a]pyridine-7-carbonitrile;
4-[(2S,6R)-2-[(4-amino-4-methyl-1-piperidyl)methyl]-6-methyl-morpholin-4-yl]pyrazolo[1,5-a]pyridine-7-carbonitrile;
4-[(2S,6R)-2-(2,5-diazabicyclo[2.2.2]octan-2-ylmethyl)-6-methyl-morpholin-4-yl]pyrazolo[1,5-a]pyridine-7-carbonitrile;
4-[(2S,6R)-2-(3,8-diazabicyclo[3.2.1]octan-3-ylmethyl)-6-methyl-morpholin-4-yl]pyrazolo[1,5-a]pyridine-7-carbonitrile;
4-[(2S,6R)-2-(2,9-diazaspiro[5.5]undecan-2-ylmethyl)-6-methyl-morpholin-4-yl]pyrazolo[1,5-a]pyridine-7-carbonitrile;
4-[(2S,6R)-2-(2,6-diazaspiro[3.5]nonan-6-ylmethyl)-6-methyl-morpholin-4-yl]pyrazolo[1,5-a]pyridine-7-carbonitrile;
4-[(2S,6R)-2-[(4-amino-3,3-difluoro-1-piperidyl)methyl]-6-methyl-morpholin-4-yl]pyrazolo[1,5-a]pyridine-7-carbonitrile;
4-[(2S,6R)-2-[(4-amino-3,3-difluoro-pyrrolidin-1-yl)methyl]-6-methyl-morpholin-4-yl]pyrazolo[1,5-a]pyridine-7-carbonitrile;
4-[(2S,6R)-2-[(8-amino-3-azabicyclo[3.2.1]octan-3-yl)methyl]-6-methyl-morpholin-4-yl]pyrazolo[1,5-a]pyridine-7-carbonitrile;
4-[(2S,6R)-2-[(3-amino-8-azabicyclo[3.2.1]octan-8-yl)methyl]-6-methyl-morpholin-4-yl]pyrazolo[1,5-a]pyridine-7-carbonitrile;
4-[(2R,6S)-2-methyl-6-[[(3R)-3-phenylpiperazin-1-yl]methyl]morpholin-4-yl]pyrazolo[1,5-a]pyridine-7-carbonitrile;
4-[(2R,6S)-2-methyl-6-[(4-piperazin-1-yl-1-piperidyl)methyl]morpholin-4-yl]pyrazolo[1,5-a]pyridine-7-carbonitrile;
4-[(2S,6R)-2-(4,7-diazaspiro[2.5]octan-7-ylmethyl)-6-methyl-morpholin-4-yl]pyrazolo[1,5-a]pyridine-7-carbonitrile;
4-[(2S,6R)-2-(1,2,3,3a,4,6,7,7a-octahydropyrrolo[3,2-c]pyridin-5-ylmethyl)-6-methyl-morpholin-4-yl]pyrazolo[1,5-a]pyridine-7-carbonitrile;
4-[(2S,6R)-2-(2,6-diazaspiro[4.5]decan-2-ylmethyl)-6-methyl-morpholin-4-yl]pyrazolo[1,5-a]pyridine-7-carbonitrile;
4-[(2S,6R)-2-(3,8-diazabicyclo[3.2.1]octan-8-ylmethyl)-6-methyl-morpholin-4-yl]pyrazolo[1,5-a]pyridine-7-carbonitrile;
4-[(2S,6R)-2-(2,7-diazaspiro[4.5]decan-7-ylmethyl)-6-methyl-morpholin-4-yl]pyrazolo[1,5-a]pyridine-7-carbonitrile;
4-[(2S,6R)-2-(1,2,3,4,4a,5,7,7a-octahydropyrrolo[3,4-b]pyridin-6-ylmethyl)-6-methyl-morpholin-4-yl]pyrazolo[1,5-a]pyridine-7-carbonitrile;
4-[(2S,6R)-2-(2,8-diazaspiro[3.6]decan-2-ylmethyl)-6-methyl-morpholin-4-yl]pyrazolo[1,5-a]pyridine-7-carbonitrile;
4-[(2S,6R)-2-(1,9-diazaspiro[5.5]undecan-9-ylmethyl)-6-methyl-morpholin-4-yl]pyrazolo[1,5-a]pyridine-7-carbonitrile;
4-[(2S,6R)-2-(1,7-diazaspiro[3.5]nonan-7-ylmethyl)-6-methyl-morpholin-4-yl]pyrazolo[1,5-a]pyridine-7-carbonitrile;
4-[(2S,6R)-2-(1,3,3a,4,5,6,7,7a-octahydropyrrolo[3,4-c]pyridin-2-ylmethyl)-6-methyl-morpholin-4-yl]pyrazolo[1,5-a]pyridine-7-carbonitrile;
4-[(2R,6S)-2-methyl-6-[(2-methylpiperazin-1-yl)methyl]morpholin-4-yl]pyrazolo[1,5-a]pyridine-7-carbonitrile;
4-[(2S,6R)-2-[(4-amino-2-methyl-1-piperidyl)methyl]-6-methyl-morpholin-4-yl]pyrazolo[1,5-a]pyridine-7-carbonitrile;
4-[(2S,6R)-2-(3,7-diazabicyclo[4.2.0]octan-7-ylmethyl)-6-methyl-morpholin-4-yl]pyrazolo[1,5-a]pyridine-7-carbonitrile;
4-[(2S,6R)-2-(1,8-diazaspiro[4.5]decan-8-ylmethyl)-6-methyl-morpholin-4-yl]pyrazolo[1,5-a]pyridine-7-carbonitrile;
4-[(2R,6S)-2-methyl-6-[(4-pyrrolidin-1-yl-1-piperidyl)methyl]morpholin-4-yl]pyrazolo[1,5-a]pyridine-7-carbonitrile;
4-[(2S,6R)-2-(1,6-diazaspiro[3.3]heptan-1-ylmethyl)-6-methyl-morpholin-4-yl]pyrazolo[1,5-a]pyridine-7-carbonitrile;
4-[(2S,6R)-2-(3,8-diazabicyclo[4.2.0]octan-8-ylmethyl)-6-methyl-morpholin-4-yl]pyrazolo[1,5-a]pyridine-7-carbonitrile;
4-[(2R,6S)-2-methyl-6-(3-oxa-7,9-diazabicyclo[3.3.1]nonan-9-ylmethyl)morpholin-4-yl]pyrazolo[1,5-a]pyridine-7-carbonitrile;
4-[(2S,6R)-2-[(7-amino-3-oxa-9-azabicyclo[3.3.1]nonan-9-yl)methyl]-6-methyl-morpholin-4-yl]pyrazolo[1,5-a]pyridine-7-carbonitrile;
4-[(2S,6R)-2-[(2-benzylpiperazin-1-yl)methyl]-6-methyl-morpholin-4-yl]pyrazolo[1,5-a]pyridine-7-carbonitrile;
4-[(2R,6S)-2-methyl-6-(I-oxa-4,9-diazaspiro[5.5]undecan-9-ylmethyl)morpholin-4-yl]pyrazolo[1,5-a]pyridine-7-carbonitrile;
4-[(2S,6R)-2-(2,3,4,4a,5,6,7,7a-octahydropyrrolo[3,4-b]pyridin-1-ylmethyl)-6-methyl-morpholin-4-yl]pyrazolo[1,5-a]pyridine-7-carbonitrile;
4-[(2R,6S)-2-methyl-6-[[cis-(3aR,6aR)-3,3a,4,5,6,6a-hexahydro-2H-pyrrolo[3,4-b]pyrrol-1-yl]methyl]morpholin-4-yl]pyrazolo[1,5-a]pyridine-7-carbonitrile;
4-[(2R,6S)-2-methyl-6-[[cis-(3aS,7aS)-1,2,3,3a,5,6,7,7a-octahydropyrrolo[3,2-b]pyridin-4-yl]methyl]morpholin-4-yl]pyrazolo[1,5-a]pyridine-7-carbonitrile;
4-[(2S,6R)-2-(3,4,4a,5,6,7,8,8a-octahydro-2H-1,5-naphthyridin-1-ylmethyl)-6-methyl-morpholin-4-yl]pyrazolo[1,5-a]pyridine-7-carbonitrile;
Endo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile;
Exo-4-[(2S,6R)-2-[(3-amino-9-azabicyclo[3.3.1]nonan-9-yl)methyl]-6-methyl-morpholin-4-yl]pyrazolo[1,5-a]pyridine-7-carbonitrile;
4-[(2S,6R)-2-(2,9-diazaspiro[5.5]undecan-2-ylmethyl)-6-methyl-morpholin-4-yl]-3-fluoro-pyrazolo[1,5-a]pyridine-7-carbonitrile;
3-fluoro-4-[(2S,6R)-2-(2,7-diazaspiro[4.5]decan-7-ylmethyl)-6-methyl-morpholin-4-yl]pyrazolo[1,5-a]pyridine-7-carbonitrile;
3-fluoro-4-[(2S,6R)-2-(2,9-diazaspiro[4.5]decan-2-ylmethyl)-6-methyl-morpholin-4-yl]pyrazolo[1,5-a]pyridine-7-carbonitrile;
4-[(2S,6R)-2-(3,7-diazabicyclo[4.2.0]octan-3-ylmethyl)-6-methyl-morpholin-4-yl]pyrazolo[1,5-a]pyridine-7-carbonitrile;
4-[(2R,6S)-2-methyl-6-(l-oxa-4,9-diazaspiro[5.5]undecan-4-ylmethyl)morpholin-4-yl]pyrazolo[1,5-a]pyridine-7-carbonitrile;
1-[[(2S,6R)-4-(7-cyanopyrazolo[1,5-a]pyridin-4-yl)-6-methyl-morpholin-2-yl]methyl]piperidine-3-carboxamide;
4-[(2S,6R)-2-[(4-amino-4-phenyl-1-piperidyl)methyl]-6-methyl-morpholin-4-yl]pyrazolo[1,5-a]pyridine-7-carbonitrile;
4-[(2S,6R)-2-[[(3R,4S)-4-amino-3-methyl-1-piperidyl]methyl]-6-methyl-morpholin-4-yl]pyrazolo[1,5-a]pyridine-7-carbonitrile;
4-[(2S,6R)-2-[[4-(dimethylamino)-3,3a,4,5,6,6a-hexahydro-1H-cyclopenta[c]pyrrol-2-yl]methyl]-6-methyl-morpholin-4-yl]pyrazolo[1,5-a]pyridine-7-carbonitrile;
4-[(2R,6S)-2-methyl-6-[(9-methyl-6-oxa-2,9-diazaspiro[4.5]decan-2-yl)methyl]morpholin-4-yl]pyrazolo[1,5-a]pyridine-7-carbonitrile; and
4-[(2S,6R)-2-[[2-benzyl-4-(4-piperidyl)piperazin-1-yl]methyl]-6-methyl-morpholin-4-yl]pyrazolo[1,5-a]pyridine-7-carbonitrile;
or a pharmaceutically acceptable salt, enantiomer or diastereomer thereof.

13. A process for the preparation of a compound according to any one of claims 1 to 12 comprising any of the following steps:
a) the reaction of compound of formula (VI), 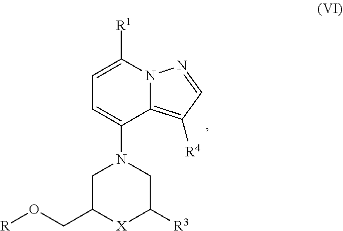
with amine (VII) in the presence of a base;
wherein the base is K₂CO₃, DIPEA or Cs₂CO₃; R is Ms, Ts or Tf; R¹, R³, R⁴ and X are defined as in any one of claims 1 to 11.

14. A compound or pharmaceutically acceptable salt, enantiomer or diastereomer according to any one of claims 1 to 12 for use as therapeutically active substance.

15. A pharmaceutical composition comprising a compound in accordance with any one of claims 1 to 12 and a therapeutically inert carrier.

16. The use of a compound according to any one of claims 1 to 12 for the treatment or prophylaxis of systemic lupus erythematosus or lupus nephritis.

17. The use of a compound according to any one of claims 1 to 12 for the preparation of a medicament for the treatment or prophylaxis of systemic lupus erythematosus or lupus nephritis.

18. The use of a compound according to any one of claims 1 to 12 as the TLR7 or TLR8 or TLR9 antagonist.

19. The use of a compound according to any one of claims 1 to 12 as the TLR7 and TLR8 antagonist.

20. A compound or pharmaceutically acceptable salt, enantiomer or diastereomer according to any one of claims 1 to 12 for the treatment or prophylaxis of systemic lupus erythematosus or lupus nephritis.

21. A compound or pharmaceutically acceptable salt, enantiomer or diastereomer according to any one of claims 1 to 12, when manufactured according to a process of claim 13.

22. A method for the treatment or prophylaxis of systemic lupus erythematosus or lupus nephritis, which method comprises administering a therapeutically effective amount of a compound as defined in any one of claims 1 to 12.

23. The invention as hereinbefore described.
